(19) United States
(12) Patent Application Publication
(10) Pub. No.: US 2010/0331233 A1
(43) Pub. Date: Dec. 30, 2010

(54) CRYSTAL OF XPA AND ERCC1 COMPLEX AND USES THEREOF

(76) Inventors: Orlando D. Schärer, Stony Brook, NY (US); Tom Ellenberger, Villa Ridge, MO (US); Oleg V. Tsodikov, Dexter, MI (US)

Correspondence Address: KENYON & KENYON LLP, ONE BROADWAY, NEW YORK, NY, 10004, US

(21) Appl. No.: 12/681,279

(22) PCT Filed: Oct. 4, 2008
(86) PCT No.: PCT/US08/11526

§ 371 Date: Sep. 15, 2010

Related U.S. Application Data
(60) Provisional application No. 60/978,011, filed on Oct. 5, 2007.

(51) Int. Cl. A61K 38/00; G01N 33/566; C07K 7/00; C07K 14/435; C07K 7/08; C07K 7/06; A61P 35/00

(52) U.S. Cl. 514/1.1; 436/501; 530/300; 530/324; 530/325; 530/326; 530/327; 530/328; 530/350

(57) ABSTRACT

The present invention relates to complexes and crystals of an XPA peptide and an ERCC1 peptide, and structural coordinates of the complex obtained from such crystals. The coordinates are useful for identifying compounds that bind to ERCC1 and inhibit binding of XPA, and thus inhibitors of nucleotide excision repair (NER). The NER inhibitors are used for treating neoplastic diseases, cancer, and hyperproliferative disorders.

### CROSS REFERENCE TO RELATED APPLICATIONS

This application claims priority to U.S. Application No. 60/978,011, filed Oct. 5, 2007, which is incorporated herein by reference in its entirety.

### FEDERAL FUNDING

This invention was produced in part using funds obtained through grants R01 GM52504 (NIGMS), P01 CA092584 (NCI) and 2P01CA092584-06 (NCI) from the National Institutes of Health. Consequently, the federal government has certain rights in this invention.

### FIELD OF THE INVENTION

The present invention relates to complexes and crystals of an XPA peptide and an ERCC1 peptide, and structural coordinates of the complex obtained from such crystals. The coordinates are useful for identifying compounds that bind to ERCC1 and inhibit binding of XPA, and thus inhibitors of nucleotide excision repair (NER). The NER inhibitors are used for treating neoplastic diseases, cancer, and hyperproliferative disorders.

### BACKGROUND OF THE INVENTION

The repair of chemical insults to DNA caused by UV light and other mutagens is essential for coping successfully with the intrinsic reactivity of DNA and preserving genetic information. Inherited diseases resulting in the failure to correct spontaneous or environmentally-induced damage are typically associated with genomic instability and a predisposition to various cancers (Friedberg et al., 2005, DNA Repair and Mutagenesis. ASM Press, Washington, D.C.). Contrarily, DNA repair is undesirable when DNA damaging agents are used for chemotherapy of cancer and other diseases. In these settings, the ability to modulate the DNA repair activities of cells targeted for destruction is a desirable goal (Ding et al., 2006, Trends Pharmacol. Sci., 27, 338-344).

DNA repair is commonly divided into five major pathways (direct damage reversal, base excision repair, nucleotide excision repair (NER), mismatch repair, and double strand break repair), each dealing, except for some overlap, with specific types of lesions. NER is a particularly intriguing repair pathway because of its extraordinarily wide substrate specificity; it has the ability to recognize and repair a large number of structurally unrelated lesions, such as DNA damage formed upon exposure to the UV radiation from sunlight and numerous bulky DNA adducts induced by mutagenic chemicals from the environment or by cytotoxic drugs used in chemotherapy. NER operates through a “cut-and-patch” mechanism by excising and removing a short stretch of DNA (24- to 32-nucleotides long) containing the damaged base; the original genetic sequence is then restored using the nondamaged strand of the DNA double helix as a template for repair synthesis.

Nucleotide excision repair (NER) is a mechanism for removing and repairing lesions that distort the DNA helix, such as ultraviolet light-induced cyclobutane pyrimidine dimers or adducts caused by alkylating and platinating anticancer drugs. One of the most astonishing characteristics of the NER pathway is its ability to recognize and excise an extraordinary diversity of DNA damage. Lesions that are processed by NER involve one or more nucleotides, arise from modifications at different positions of the purine or pyrimidine bases, and are formed by UV irradiation or upon exposure to chemically reactive molecules.

The removal of bulky and helix-distorting DNA lesions by the NER pathway requires the coordinated assembly of a large multiprotein complex (Houtsmuller et al., 1999, Science, 284, 958-961; Volker et al., 2001, Mol. Cell, 8, 213-224) that exposes the damaged DNA strand and excises an oligonucleotide containing the lesion (Gillet and Schärer, 2006, Chem. Rev., 106, 253-276). NER involves over 30 proteins that recognize damaged sites in DNA and excise an oligonucleotide containing the damage (de Laat et al., 1999, Genes Dev., 13, 768-785; Gillet and Schärer, 2006). Following excision of the damaged DNA segment, the resulting gap is filled by templated DNA synthesis and ligase seals the nick to complete the repair. Cell biological and biochemical studies have shown that NER operates by the sequential assembly of protein factors at the sites of DNA damage, rather than through the action of a preformed repairosome (Houtsmuller et al., 1999; Volker et al., 2001). The recruitment of NER factors into protein-DNA ensembles is guided at each step by numerous protein-protein interactions (Araujo et al., 2001, Mol. Cell. Biol., 21, 2281-2291), imparting specificity to the recognition and verification of damaged sites. Damage recognition in NER culminates with the incision of DNA 5′ and 3′ to the lesion site by ERCC1-XPF and XPG, respectively, to release a 24-32 nucleotide segment containing the damage (de Laat et al., 1999; Gillet and Scharer, 2006). For the NER pathway, DNA cleavage by ERCC1-XPF requires physical interaction with XPA, a scaffold protein that interacts with DNA and several repair proteins, including RPA, TFIIH and the ERCC1 subunit of ERCC1-XPF (Li et al., 1994, Proc. Natl. Acad. Sci. USA, 91, 5012-5016; Li et al., 1995, Mol. Cell. Biol., 15, 1993-1998; Park and Sancar, 1994, Proc. Natl. Acad. Sci. USA, 91, 5017-5021; Saijo et al., 1996, Nucleic Acids Res., 24, 4719-4724). Although XPA was originally described as a DNA damage-specific sensor or verification protein, recent work suggests that XPA instead recognizes the DNA structural intermediates arising during processing by NER (Camenisch et al., 2006, Nat. Struct. Mol. Biol., 13, 278-284; Jones and Wood, 1993, Biochemistry, 32, 12096-12104). XPA recruits ERCC1-XPF to NER complexes (Volker et al., 2001), positioning the XPF nuclease domain at the 5′ side of the damage site (Enzlin and Schärer, 2002, EMBO J., 21, 2045-2053). ERCC1-XPF has other roles in DNA metabolism outside of NER, notably in interstrand crosslink repair and homologous recombination (Hoy et al., 1985, Somat. Cell. Mol. Genet., 11, 523-532; Niedernhofer et al., 2001, EMBO J., 20, 6540-6549). The importance of these additional, NER-independent functions of ERCC1-XPF is underscored by the pronounced sensitivity to crosslinking agents caused by mutations of ERCC1 or XPF in mice and humans (McWhir et al., 1993, Nat. Genet., 5, 217-224; Niedernhofer et al., 2006, Nature, 444, 1038-1043; Weeda et al., 1997, Curr. Biol., 7, 427-439). However, the exact biochemical role(s) of ERCC1-XPF in crosslink repair remain to be discovered.

Li and coworkers identified residues 59-114 in XPA as the site of interaction with ERCC1, and showed that a deletion of 3 conserved glycines (Gly72, Gly73, Gly74) abrogates the XPA-ERCC1 interaction as well as the ability of the XPA protein to confer UV resistance to XP-A cells (Li et al., 1994; Li et al., 1995). Furthermore, the expression of a truncated protein comprising residues 59-114 of XPA renders cells sensitive to UV light and cisplatin (Rosenberg et al., 2001, Cancer Res., 61, 764-770), suggesting that this region is sufficient to disrupt the interaction of the native XPA protein with ERCC1-XPF. Conversely, it can be inferred from previous studies that residues 92-119 of ERCC1 are necessary for the interaction with XPA (Li et al., 1994).

Following these seminal studies, understanding of the biochemical and structural basis for XPA's interaction with ERCC1 has not advanced, although more is known about the individual proteins. XPA contains a well-defined central domain (residues 98-219; FIG. 1A), although the remainder of the protein including the ERCC1 interaction domain appears to be poorly structured (Buchko et al., 2001, Mutat. Res., 486, 1-10; Buchko et al., 1998, Nucleic. Acids Res., 26, 2779-2788; Iakoucheva et al., 2001, Protein Sci., 10, 560-571; Ikegami et al., 1998, Nat. Struct. Biol., 5, 701-706). Residues 99-119 of ERCC1 fall within the central domain of ERCC1 (Tsodikov et al., 2005, Proc. Natl. Acad. Sci. USA, 102, 11236-11241) that is structurally homologous to the nuclease domain of the archaeal XPF-like proteins Aeropyrum pernix Mus81/XPF (Newman et al., 2005, EMBO J., 24, 895-905) and Pyrococcus furiosus Hef (Nishino et al., 2003, Structure, 11, 445-457). A V-shaped groove on the surface of ERCC1 corresponds to the nuclease active site of XPF (Enzlin and Schärer, 2002; Newman et al., 2005; Nishino et al., 2003), except that ERCC1's groove lacks the catalytic residues of a nuclease active site and is instead populated with basic and aromatic residues (Gaillard and Wood, 2001, Nucleic Acids Res., 29, 872-879). It has previously been shown that the central domain of ERCC1 binds to single-stranded DNA in vitro (Tsodikov et al., 2005), and the V-shaped groove has been proposed as the DNA binding site.

Although it is understood that XPA has a role in recruiting the XPF-ERCC1 endonuclease to sites of NER, the structural and functional basis of the interaction of XPA and ERCC1 is unknown. The present invention provides defined XPA peptides that bind to ERCC1, complexes of the peptides and ERCC1, and demonstrates the structural properties of the complexes. Further, the XPA peptides of the present invention specifically inhibit the NER reaction in vitro, creating an opportunity for structure-based design of NER inhibitors targeting this protein-protein interaction. Certain point mutations in the corresponding region in XPA abolish NER activity in vitro, underscoring the importance of XPA-ERCC1 interactions for NER. Mutant XPA peptides are also provided by the present invention. In addition to providing insights into how protein-protein interactions mediate progression through the NER pathways, the present invention provides methods for identifying mimetics, such as small molecules, that intercept the interaction between XPA and ERCC1. Such molecules are valuable for modulating the biochemical functions of ERCC1-XPF in NER and other repair pathways including DNA interstrand crosslink repair and homologous recombination by selectively modulating NER.

### SUMMARY OF THE INVENTION

The present invention provides XPA peptides, ERCC1 peptides and complexes thereof. The invention further provides a crystal of a complex of an XPA peptide and an ERCC1 peptide. The present invention also provides the structure coordinates of a complex of an XPA peptide and an ERCC1 peptide.

XPA peptides of the invention are up to about 35 amino acids and comprises SEQ ID NO:1 from amino acid 70 to amino acid 78. The XPA peptides are capable of forming non-covalent complexes with ERCC1 and fragments of ERCC1, which contain an intact XPA binding site. In one embodiment, the an XPA peptide consists essentially of SEQ ID NO:1 from about amino acid 67 to about amino acid 80. In another embodiment, an XPA peptide consists essentially of SEQ ID NO:1 from about amino acid 59 to about amino acid 93. An example of an ERCC1 peptide that contains an intact XPA binding site has SEQ ID NO:2 from amino acid 92 to amino acid 214. Another such ERCC1 peptide has SEQ ID NO:2 from about amino acid 96 to about amino acid 214.

XPA/ERCC1 complexes are useful for characterizing binding interactions between the two peptides, as well as to identify compounds that can be used to disrupt XPA/ERCC1 complex formation. One way to characterize binding interactions is by X-ray crystallography. Another way is by NMR. Accordingly, the invention provides a crystal of an XPA/ERCC1 complex that effectively diffracts X-rays for the determination of the atomic coordinates of the complex. In one embodiment, an XPA/ERCC1 complex consists essentially of an XPA peptide that contains SEQ ID NO:1 from about amino acid 67 to about amino acid 80 and ERCC1 peptide that contains SEQ ID NO:2 from about amino acid 92 to about amino acid 214. The crystal belongs to space group I4₁32 and has unit cell dimensions a=b=c=128.6 Å. In one embodiment, a crystal of the invention effectively diffracts to at least 5 Å resolution. In another embodiment, a crystal of the invention diffracts to at least 4 Å. In yet another embodiment, a crystal of the invention diffracts to at least 3 Å. Data collected in X-ray diffraction experiments can be used alone, or in combination with NMR data to provide an accurate model of atoms of the XPA/ERCC1 binding site.

The invention provides a method of preparing a crystal of an XPA/ERCC1 complex, which comprises incubating a mixture comprising an XPA central domain peptide and an ERCC1 central domain peptide in a closed container with a reservoir solution comprising a precipitant under conditions suitable for crystallization until a crystal forms. In one embodiment, the mixture comprises Tris buffer, ammonium dihydrogen phosphate, and optionally, glycerol. In an embodiment of the invention, the reservoir solution comprises ammonium dihydrogen phosphate.

The invention also provides a method of determining whether a compound inhibits the formation of an XPA/ERCC1 complex (inhibits NER activity). In one embodiment, inhibition is determined by contacting the components of an XPA/ERCC1 complex with a test compound, in vitro or in vivo, and measuring complex formation. For example, a test compound is contacted with an ERCC1 polypeptide that binds to XPA and an XPA polypeptide of up to about 35 amino acids which comprises SEQ ID NO:1 from amino acid 70 to amino acid 78 under conditions in which a complex of the XPA and ERCC1 polypeptides can form in the absence of the compound, and measuring the binding of the ERCC1 polypeptide with the XPA polypeptide. A compound is identified as an inhibitor of complex formation when there is a decrease in the binding of the ERCC1 polypeptide with the XPA polypeptide in the presence but not the absence of the test compound.

The invention also provides a method of identifying a compound which inhibits formation of an XPA/ERCC1 complex which involves providing structural coordinates defining all or a portion of the three-dimensional structure of the XPA binding site of ERCC1, providing structural coordinates of a test compound, and fitting the structure of the test compound to structural coordinates of the XPA binding site of ERCC1. In one embodiment, the structural coordinates comprise all or a portion of the coordinates of Table 1. In another embodiment, structural coordinates are provided for two or more amino acids selected from the group consisting of Arg106, Asn110, Asn129, Phe 140, Ser142, Arg144, Tyr145, Leu148, His149, Tyr152, Arg156. In another embodiment, structural coordinates are provided for at least Asn110, Ser142, Tyr145, and Tyr152. In another embodiment, structural coordinates are provided for at least Asn110, Ser142, Arg144, Tyr145, Leu148, and Tyr152. In another embodiment, structural coordinates are provided for at least Arg106, Asn110, Phe140, Ser142, Tyr145, and Tyr152. In yet another embodiment, structural coordinates are provided for at least Asn110, Asn129, Ser142, Tyr145, Tyr152, and Arg156.

The invention also provides method of identifying a compound that inhibits the formation of an XPA/ERCC1 complex (inhibits NER activity) comprising providing structural coordinates defining all or a portion of XPA when complexed with ERCC1, providing structural coordinates of the compound, and fitting the structure of the compound to XPA structural coordinates. In an embodiment of the invention, structural coordinates are provided for two or more amino acids selected from the group consisting of Thr71, Gy72, Gly73, Gly74, Phe75, and Ile76. In another embodiment, structural coordinates are provided for Gly72, Gly73, Gly74, and Phe75.

Virtual libraries of compounds can be screened with the assistance of a computer. The compounds thus identified can be synthesized and further assayed for the ability to inhibit binding of XPA to ERCC1 and to inhibit the endonuclease activity of ERCC1-XPF.

Accordingly, the invention also provides a computer-assisted method for identifying a compound that inhibits NER activity using a computer comprising a processor, a data storage system, an input device, and an output device, comprising inputting into the programmed computer the three-dimensional coordinates of a subset of the atoms of an XPA-ERCC1 complex, providing a database of chemical and peptide structures stored in the computer data storage system, selecting from the database, using computer methods, structures having a portion that is structurally similar to the criteria data set, and outputting to the output device the selected chemical structures having a portion similar to the criteria data set. The compounds identified can further be tested to determine whether they inhibit NER activity.

The compounds can be peptides or small molecules. Certain peptides that inhibit XPA-ERCC1 complex formation contain the amino acid sequence Gly-Gly-Gly or Gly-Gly-Gly-Phe or Thr-Gly-Gly-Gly-Phe-Ile. In an embodiment of the invention, such a peptide is about 26 to about 35 amino acids in length. In another embodiment, the length is about 16 to about 25 amino acids. In another embodiment, the peptide is about 8 to about 15 amino acids in length. In one embodiment, the structure of the peptide is constrained to provide a conformationally constrained loop of amino acids containing the sequence Gly-Gly-Gly or Gly-Gly-Gly-Phe. For example, the peptide can be circular, of comprise a disulfide bond.

The invention provides compounds characterized by the ability to associate with a binding pocket of ERCC1 and inhibit NER activity, wherein the binding pocket of ERCC1 is defined by the atomic coordinates of two or more amino acid residues selected from the group consisting of Arg106, Asn110, Asn129, Phe 140, Ser142, Arg144, Tyr145, Leu148, Tyr152, Arg156, and combinations thereof. The invention further provides compounds that comprise atoms having locations that correspond to atoms of XPA in a complex with ERCC1.

Further provided are methods and compositions for inhibiting NER activity, inhibiting tumor growth in a mammal, and treating a hyperproliferative disease by administering an effective amount of compounds identified by the methods disclosed herein. For inhibition of tumor growth, the compounds can be administered alone or in combination with other anti-neoplastic agents.

### BRIEF DESCRIPTION OF THE FIGURES

FIG. 1 illustrates the XPA domain organization and structure of the ERCC1-binding peptide. (A) The ERCC1-binding region of XPA (residues 67-77) is located between the central domain (Zn²⁺-binding and DNA-binding subdomains; residues 98-219) and an N-terminal region (residues 1-58) that is dispensable for functional complementation of NER in whole cell extracts from XP-A mutant cells (Miyamoto et al., 1992, J. Biol. Chem., 267, 12182-12187) and a TFIIH-binding region (Park et al., 1995, J. Biol. Chem., 270, 4896-4902). (B) ¹⁵N HSQC spectrum of ¹⁵N-labeled XPA₅₉₋₉₃ in complex with unlabeled ERCC1, and in the unbound state (inset). The spectrum of the unbound XPA₅₉₋₉₃ (inset) is characteristic of an unfolded peptide. The appearance of new well-dispersed NMR peaks in the XPA spectrum upon addition of ERCC1₉₂₋₂₁₄ (shown in the larger spectrum) indicates that a portion of the XPA peptide adopts a defined conformation in complex with ERCC1.

FIG. 2 illustrates the structure of the XPA-ERCC1 complex. (A) The XPA₆₇₋₈₀ peptide (orange) is bound to a V-shaped groove of the central domain of ERCC1₉₆₋₂₁₄ (green). An orthogonal view of the bound XPA peptide (left side) is shown in comparison to the peptide in complex with ERCC1 (right side). (B) The XPA binding site on the surface of ERCC1 (colored red) was identified by resonance perturbations larger than 0.2 ppm that are indicative of direct interactions with XPA.

FIG. 3 illustrates the binding of XPA₆₇₋₈₀ in a shallow groove of ERCC1. (A) A comparison of the two dimensional HSQC spectra for ¹⁵N-labeled ERCC1₉₂₋₂₁₄ in the presence and absence of an unlabeled XPA₆₇₋₈₀ peptide. The ¹⁵N HSQC spectra reveal significant chemical shift changes for some ERCC1 residues (labeled) in the absence or presence of unlabeled XPA₆₇₋₈₀. (B) Combined average chemical shift perturbations are calculated as Δχₐᵥₑ=[((Δχ_{1H})²+(Δχ_{15N}/5)²)/2)]^{1/2} for each backbone amide of ERCC1 and shown as a histogram.

FIG. 4 illustrates that the XPA₆₇₋₈₀ peptide is an effective inhibitor of NER activity. (A) XPA₆₇₋₈₀ inhibits the in vitro NER reaction, whereas the mutant XPA₆₇₋₈₀^{F75A} peptide has no effect. HeLa cell extracts were incubated with a plasmid containing a 1,3-intrastrand cisplatin adduct in the presence of increasing concentrations of either XPA₆₇₋₈₀ or XPA₆₇₋₈₀^{F75A} (lane 1, no XPA; lanes 2 and 7, 46 nM XPA peptide; lanes 3 and 8, 460 nM; lanes 4 and 9, 4.6 μM; lanes 5 and 10, 46 μM; lanes 6 and 11, 92 μM). Products were visualized by a fill in reaction following annealing to an oligonucleotide complementary to the excision product with a 4 nt overhang. (Shivji, 1999, Methods Mol. Biol., 113, 373-392). The marker DNA ladder is labeled LMW DNA ladder. (B) XPA₆₇₋₈₀ and XPA₆₇₋₈₀^{F75A} do not affect the intrinsic nuclease activity of ERCC1-XPF. An ERCC1 substrate nucleic acid having a 12 bp stem and 22 base loop (6.6 nM) was incubated with different concentrations of ERCC1-XPF (lanes 2, 4 and 6: 6.7 nM ERCC1-XPF; lanes 3, 5 and 7, 26.8 nM) and 0.4 mM MnCl₂ in the presence of no peptide (lanes 1-3), 92 μM XPA₆₇₋₈₀ so (lanes 4 and 5), and 92 μM XPA₆₇₋₈₀^{F75A} (lanes 6 and 7). The DNA substrate and the cleavage products are indicated.

FIG. 5 illustrates that mutation of the ERCC1-binding epitope of XPA abolishes NER but not DNA binding activity. (A) XP-A (XP2OS) cell extracts were incubated with a plasmid containing a 1,3-intrastrand cisplatin adduct in the presence of wild-type XPA (XPA-WT) or mutant XPA proteins (XPA-F75A, XPA-G73Δ or XPA-G73Δ/G74Δ). The reaction products were visualized by a fill in reaction after annealing the excision product to an complementary oligonucleotide with a 4 nt overhang (Shivji, 1999). Different XPA concentrations of 200 nM (lanes 1, 3, 5 and 7) and 800 nM (lanes 2, 4, 6 and 8) were tested. The position of a 25mer of the LMW DNA ladder is indicated. (B) A 5′-labeled DNA three-way junction (1 nM) was incubated with wild-type and mutant XPA proteins for 30 min at room temperature then the XPA-bound (xd) and free DNA (d) oligonucleotides were separated on an 8% native polyacrylamide gel. The reaction products generated with different concentrations of XPA are shown: 0 (lane 1), 4 nM (lanes 2, 7, 13, 17), 10 nM (lanes 3, 8, 13, 18), 25 nM (lanes 4, 9, 14, 19), 60 nM (lanes 5, 10, 15, 20), 150 nM (lanes 6, 11, 16, 21).

FIG. 6 illustrates that XPA₅₉₋₂₁₃ forms a stable 1:1 complex with the central domain of ERCC1. (A) The complex of XPA₅₉₋₂₁₃ with ERCC1₉₂₋₂₁₄ was separated by gel filtration chromatography (S-100 column; Pharmacia) from an excess of ERCC1₉₂₋₂₁₄. The XPA-ERCC1 complex (peak 1) elutes before the unbound ERCC1 (peak 2). (B) SDS-PAGE analysis of fractions corresponding to peaks 1 and 2 from the gel filtration experiment demonstrates the presence in peak 1 of XPA and ERCC1 at an approximately equimolar ratio of the two proteins. (C) Sedimentation equilibrium analysis of ERCC1₉₂₋₂₁₄ (the central domain of ERCC1) alone and in complex with XPA₅₉₋₉₃. The natural logarithm of the absorbance at 280 nm is plotted versus the square of the relative radial position. The data for unbound ERCC1₉₂₋₂₁₄ (open circles) and its complex with XPA₅₉₋₉₃ (open triangles) are plotted in a similar matter. The curves represent the best fit of these data to Equation 1, yielding molecular weights of 15 kDa for unbound ERCC1₉₂₋₂₁₄ and 19.4 kDa for ERCC1₉₂₋₂₁₄-XPA₅₉₋₉₃, in good agreement with the prediction for a 1:1 binding stoichiometry.

FIG. 7 illustrates that the XPA₆₇₋₈₀ peptide is a competitive inhibitor of single-stranded DNA binding to ERCC1₉₂₋₂₁₄. Binding of a 6FAM-labeled single-stranded DNA 40mer to the central domain of ERCC1 was monitored by fluorescence polarization of the labeled DNA. DNA binding activity is plotted as a function of added XPA₆₇₋₈₀ inhibitor. The theoretical curve given by Eq. 9 (solid line) corresponds to the best fit of the data to the dissociation equilibrium constant of 300 nM for the ERCC1-XPA complex.

### DESCRIPTION OF THE INVENTION

The present invention provides XPA peptides and complexes with ERCC1, and crystals of an XPA-ERCC1 complex. XPA peptides that bind to ERCC1 generally include the amino acids of SEQ ID NO:1 from about amino acid 70 to amino acid 78. Accordingly, XPA peptides of the invention comprise up to about 35 amino acids of XPA, including the amino acids of SEQ ID NO:1 from amino acid 70 to amino acid 78. In one embodiment of the invention, the XPA fragment consists essentially of SEQ ID NO:1 from about amino acid 67 to about amino acid 80. In another embodiment of the invention an XPA peptide consists essentially of SEQ ID NO:1 from about amino acid 59 to about amino acid 93. The peptide fragments may be obtained as described in the examples. For example, the peptides may be synthesized by solid phase synthesis methods, or cloned into a vector, and expressed in a host cell.

The invention also provides complexes of the XPA peptides with ERCC1 or ERCC1 fragments that are capable of binding XPA. In one embodiment, an ERCC1 fragment that binds to XPA comprises SEQ ID NO:2 from about amino acid 59 to amino acid 93. In another embodiment, an ERCC1 fragment that binds to XPA consists essentially of SEQ ID NO:2 from about amino acid 92 to amino acid 214. In yet another embodiment, an ERCC1 fragment that binds to XPA consists essentially of SEQ ID NO:2 from about amino acid 96 to amino acid 214.

The invention provides a crystalline complex of an XPA peptide and an ERCC1 peptide. The crystal of the invention effectively diffracts X-rays for the determination of the atomic coordinates of the complex. In one embodiment, the atomic coordinates of the binding site of the complex can be determined to a resolution about 5 Å or better. In another embodiment, the atomic coordinates of the binding site of the complex can be determined to a resolution about 4 Å or better. In yet another embodiment, the atomic coordinates of the binding site of the complex can be determined to a resolution about 3 Å or better. One crystal of the invention belongs to space group I4₁32 and has unit cell dimensions a=b=c=128.6 Å.

An XPA-ERCC1 complex of the invention is represented by the atomic coordinates of Table 2. The coordinates correspond to amino acids 67 to 77 of XPA and amino acids 99 to 214 of ERCC1. The XPA-ERCC1 complex of Table 1 was determined from a complex of an XPA peptide consisting of XPA amino acids 67 to 80 and an ERCC1 central domain fragment consisting of amino acids 92-214 preceded by an N-terminal hexahistidine tag (MGSSHHHHHHSQDP; SEQ ID NO:3).

The crystals of the invention include native crystals and heavy-atom derivative crystals. Native crystals generally comprise substantially pure polypeptides corresponding to XPA peptide fragments and ERCC1 peptide fragments complexed in crystalline form. The crystal of the invention is not limited to wild-type XPA peptide fragments and ERCC1 peptide fragments. Indeed, the crystals may comprise mutants of wild-type XPA peptide fragments and/or ERCC1 peptide fragments. Mutant XPA peptide fragments and/or ERCC1 peptide fragments are obtained by replacing at least one amino acid residue in the sequence of the wild-type peptide with a different amino acid residue, or by adding or deleting one or more amino acid residues within the wild-type sequence and/or at the N- and/or C-terminus of the wild-type peptide. It is expected that mutant peptides that form a XPA-ERCC1 complex can be crystallized, for example, under crystallization conditions that are substantially similar to those used to crystallize the wild-type XPA-ERCC1 complex described herein.

Sequence alignments of polypeptides in a protein family or of homologous polypeptide domains can be used to identify potential amino acid residues in the polypeptide sequence that are candidates for mutation. Identifying mutations that do not significantly interfere with the three-dimensional structure of the binding region of an XPA/ERCC1 complex will depend, in part, on the region in the XPA and/or ERCC1 peptide fragment where the mutation occurs.

Conservative amino acid substitutions are preferred and are well-known in the art. These include substitutions made on the basis of a similarity in polarity, charge, solubility, size, hydrophobicity and/or the hydrophilicity of the amino acid residues involved. Typical conservative substitutions are those in which the amino acid is substituted with a different amino acid that is a member of the same class or category, as those classes are defined herein. Thus, typical conservative substitutions include aromatic to aromatic, apolar to apolar, aliphatic to aliphatic, acidic to acidic, basic to basic, polar to polar, etc. It will be recognized by those of skill in the art that generally, a total of about 20% or fewer, typically about 10% or fewer, most usually about 5% or fewer, of the amino acids in the wild-type polypeptide sequence can be conservatively substituted with other amino acids without deleteriously affecting the biological activity and/or three-dimensional structure of the molecule.

The heavy-atom derivative crystals from which the atomic structure coordinates of the invention can be obtained generally comprise a crystalline XPA-ERCC1 complex in association with one or more heavy metal atoms. There are two types of heavy-atom derivatives of polypeptides: heavy-atom derivatives resulting from exposure of the protein to a heavy metal in solution, wherein crystals are grown in medium comprising the heavy metal, or in crystalline form, wherein the heavy metal diffuses into the crystal, and heavy-atom derivatives wherein the polypeptide comprises heavy-atom containing amino acids, e.g., selenomethionine and/or selenocysteine mutants.

In practice, heavy-atom derivatives of the first type can be formed by soaking a native crystal in a solution comprising heavy metal atom salts, or organometallic compounds, e.g., lead chloride, gold thiomalate, ethylmercurithiosalicylic acid-sodium salt (thimerosal), uranyl acetate, platinum tetrachloride, osmium tetraoxide, zinc sulfate, and cobalt hexamine, which can diffuse through the crystal and bind to the crystalline polypeptide.

Heavy-atom derivatives of this type can also be formed by adding to a crystallization solution comprising the protein complex to be crystallized an amount of a heavy metal atom salt, which may associate with the protein complex and be incorporated into the crystal. The location(s) of the bound heavy metal atom(s) can be determined by X-ray diffraction analysis of the crystal. This information, in turn, is used to generate the phase information needed to construct the three-dimensional structure of the protein complex.

Crystallization of the XPA-ERCC1 complex may be carried out from a solution of XPA peptide and ERCC1 peptide using a variety of techniques known in the art of protein crystallography, including batch, liquid bridge, dialysis, and vapor diffusion methods (see, e.g., McPherson, 1982, Preparation and Analysis of Protein Crystals, John Wiley, New York; McPherson, 1990, Eur. J. Biochem. 189, 1-23; Weber, 1991, Adv. Protein Chem. 41, 1-36.).

Preferably, crystals of the XPA-ERCC1 complex are formed by crystallization from a solution of substantially pure XPA peptide and ERCC1 peptide. In order that an XPA-ERCC1 complex is formed, the ERCC1 peptide will generally include the ERCC1 central domain, or at least a portion of the ERCC1 central domain that contains the XPA binding site. Likewise, the XPA peptide will contain at least the portion of XPA that binds to ERCC1, such that a stable complex is formed. Often, the selected peptides will be engineered to include a tag, such as a histidine residues at the amino terminus which can be used for purification using a nickel chelation column. Suitable crystallization buffers will depend, to some extent, on the chosen XPA and ERCC1 peptides. Examples of suitable buffers include Tris, CHES, Hepes, MES, and acetate. The buffer system may be manipulated by addition of a salt, such as sodium chloride, calcium chloride, ammonium sulfate, sodium/potassium phosphate, or ammonium acetate. The concentration of the salt is about 20 mM to about 500 mM, and can be from about 25 mM to about 100 mM, and optionally about 50 mM. In certain embodiments, the pH of the buffer is preferably about 6 to about 10, more preferably about 8 to about 9.5 Crystallization matrices that provide a wide variety of crystallization formulations are well known in the art.

Preferably, the crystal is precipitated by contacting the solution with a reservoir that reduces the solubility of the proteins in the buffer due to the presence of precipitants, i.e., reagents that induce precipitation. In a preferred embodiment, contacting is carried out by vapor diffusion. (McPherson, 1982; McPherson, 1990). In this method, exemplified in the examples, the polypeptide/precipitant solution is allowed to equilibrate in a closed container with a larger buffer reservoir having a precipitant concentration optimal for producing crystals. Generally, about 1 μL of substantially pure polypeptide solution is mixed with an equal volume of reservoir solution, giving a precipitant concentration about half that required for crystallization. This solution is suspended as a droplet underneath a coverslip, which is sealed onto the top of the reservoir. The sealed container is allowed to stand, usually for about 2-6 weeks, until crystals grow.

Examples of precipitants include ammonium dihydrogen phosphate, polyethylene glycol, ammonium sulfate, 2-methyl-2,4-pentanediol, sodium citrate, sodium chloride, glycerol, isopropanol, lithium sulfate, sodium acetate, sodium formate, potassium sodium tartrate, ethanol, hexanediol, ethylene glycol, dioxane, t-butanol and combinations thereof. Precipitants operate by various mechanisms. For example, some precipitants may act by making the buffer pH unfavorable for protein solubility. Other precipitants increase the effective protein concentration by binding water molecules. Precipitation may be carried out in the presence of a heavy metal such as cadmium to assist analysis of the crystal after precipitation.

In one embodiment, illustrated in the examples, an XPA-ERCC1 complex is concentrated to 9 mg/ml in 30 mM Tris pH 8.0, 200 nM beta-mercaptoethanol and 0.1 mM EDTA. For crystals from which the atomic structure coordinates of the invention are obtained, it has been found that hanging drops, containing 1 μl of the protein solution and 1 μl of a reservoir solution (100 mM Tris pH 8.5, 2 M ammonium dihydrogen phosphate, 10% glycerol) at 21.5° C., produce diffraction quality crystals in 1-2 weeks.

The dimensions of a unit cell of a crystal are defined by six numbers, the lengths of three unique edges, a, b, and c, and three unique angles, α, β, and γ. The type of unit cell that comprises a crystal is dependent on the values of these variables. When a crystal is placed in an X-ray beam, the incident X-rays interact with the electron cloud of the molecules that make up the crystal, resulting in X-ray scatter. The combination of X-ray scatter with the lattice of the crystal gives rise to nonuniformity of the scatter; areas of high intensity are called diffracted X-rays. The angle at which diffracted beams emerge from the crystal can be computed by treating diffraction as if it were reflection from sets of equivalent, parallel planes of atoms in a crystal (Bragg's Law). The most obvious sets of planes in a crystal lattice are those that are parallel to the faces of the unit cell. These and other sets of planes can be drawn through the lattice points. Each set of planes is identified by three indices, hkl. The h index gives the number of parts into which the a edge of the unit cell is cut, the k index gives the number of parts into which the b edge of the unit cell is cut, and the l index gives the number of parts into which the c edge of the unit cell is cut by the set of hkl planes. Thus, for example, the 235 planes cut the a edge of each unit cell into halves, the b edge of each unit cell into thirds, and the c edge of each unit cell into fifths. Planes that are parallel to the be face of the unit cell are the 100 planes; planes that are parallel to the ac face of the unit cell are the 010 planes; and planes that are parallel to the ab face of the unit cell are the 001 planes.

When a detector is placed in the path of the diffracted X-rays, in effect cutting into the sphere of diffraction, a series of spots, or reflections, are recorded to produce a “still” diffraction pattern. Each reflection is the result of X-rays reflecting off one set of parallel planes, and is characterized by an intensity, which is related to the distribution of molecules in the unit cell, and hkl indices, which correspond to the parallel planes from which the beam producing that spot was reflected. If the crystal is rotated about an axis perpendicular to the X-ray beam, a large number of reflections is recorded on the detector, resulting in a diffraction pattern.

The unit cell dimensions and space group of a crystal can be determined from its diffraction pattern. First, the spacing of reflections is inversely proportional to the lengths of the edges of the unit cell. Therefore, if a diffraction pattern is recorded when the X-ray beam is perpendicular to a face of the unit cell, two of the unit cell dimensions may be deduced from the spacing of the reflections in the x and y directions of the detector, the crystal-to-detector distance, and the wavelength of the X-rays. Those of skill in the art will appreciate that, in order to obtain all three unit cell dimensions, the crystal must be rotated such that the X-ray beam is perpendicular to another face of the unit cell. Second, the angles of a unit cell can be determined by the angles between lines of spots on the diffraction pattern. Third, the absence of certain reflections and the repetitive nature of the diffraction pattern, which may be evident by visual inspection, indicate the internal symmetry, or space group, of the crystal. Therefore, a crystal may be characterized by its unit cell and space group, as well as by its diffraction pattern. Because the lengths of the unit cell axes in a protein crystal are large and the concomitant reciprocal cell lengths are very short, the unit cell dimensions and space group of a protein crystal can be determined from one reciprocal space photograph if the crystal is rotated through approximately one degree.

Once the dimensions of the unit cell are determined, the likely number of polypeptides in the asymmetric unit can be deduced from the size of the polypeptide, the density of the average protein, and the typical solvent content of a protein crystal, which is usually in the range of 30-70% of the unit cell volume (Matthews, 1968, J. Mol. Biol. 33, 491-497).

The diffraction pattern is related to the three-dimensional shape of the molecule by a Fourier transform. The process of determining the solution is in essence a re-focusing of the diffracted X-rays to produce a three-dimensional image of the molecule in the crystal. Since re-focusing of X-rays cannot be done with a lens at this time, it is done via mathematical operations.

The sphere of diffraction has symmetry that depends on the internal symmetry of the crystal, which means that certain orientations of the crystal will produce the same set of reflections. Thus, a crystal with high symmetry has a more repetitive diffraction pattern, and there are fewer unique reflections that need to be recorded in order to have a complete representation of the diffraction. The goal of data collection, a dataset, is a set of consistently measured, indexed intensities for as many reflections as possible. A complete dataset is collected if at least 80%, preferably at least 90%, most preferably at least 95% of unique reflections are recorded. In one embodiment, a complete dataset is collected using one crystal. In another embodiment, a complete dataset is collected using more than one crystal of the same type.

Sources of X-rays include, but are not limited to, a rotating anode X-ray generator such as an Elliott GX13 or a beamline at a synchrotron light source, such as the X8C beamline at the National Synchrotron Light Source at Brookhaven National Laboratory. Suitable detectors for recording diffraction patterns include, but are not limited to, X-ray sensitive film, multiwire area detectors, image plates coated with phosphorus, and CCD cameras. Typically, the detector and the X-ray beam remain stationary, so that, in order to record diffraction from different parts of the crystal's sphere of diffraction, the crystal itself is moved via an automated system of moveable circles called a goniostat.

Once a dataset is collected, the information is used to determine the three-dimensional structure of the molecule in the crystal. However, this cannot be done from a single measurement of reflection intensities because certain information, known as phase information, is lost between the three-dimensional shape of the molecule and its Fourier transform, the diffraction pattern. This phase information must be acquired in order to perform a Fourier transform on the diffraction pattern to obtain the three-dimensional structure of the molecule in the crystal. It is the determination of phase information that in effect refocuses X-rays to produce the image of the molecule.

One method of obtaining phase information is by molecular replacement, which is a method of calculating initial phases for a new crystal of a polypeptide whose structure coordinates are unknown by orienting and positioning a polypeptide whose structure coordinates are known, and believed to be similar to the polypeptide of unknown structure, within the unit cell of the new crystal so as to best account for the observed diffraction pattern of the new crystal. Phases are then calculated from the oriented and positioned polypeptide and combined with observed amplitudes to provide an approximate Fourier synthesis of the structure of the molecules comprising the new crystal. (Lattman, 1985, Methods Enzymol. 115, 55-77; Rossmann, 1972, “The Molecular Replacement Method,” Int. Sci. Rev. Ser. No. 13, Gordon & Breach, New York; Brunger et al., 1991, Acta Crystallogr. A. 47, 195-204).

Once phase information is obtained, it is combined with the diffraction data to produce an electron density map, an image of the electron clouds that surround the atoms of the molecule(s) in the unit cell. The higher the resolution of the data, the more distinguishable are the features of the electron density map, e.g., amino acid side chains and the positions of carbonyl oxygen atoms in the peptide backbones, because atoms that are closer together are resolvable. A model of the macromolecule is then built into the electron density map with the aid of a computer, using as a guide all available information, such as the polypeptide sequence and the established rules of molecular structure and stereochemistry. Interpreting the electron density map is a process of finding the chemically realistic conformation that fits the map precisely.

After a model is generated, a structure is refined. Refinement is the process of minimizing the average of the differences between observed structure factors (square-root of intensity) and calculated structure factors which are a function of the position, temperature factor and occupancy of each non-hydrogen atom in the model. This usually involves alternate cycles of real space refinement, i.e., calculation of electron density maps and model building, and reciprocal space refinement, i.e., computational attempts to improve the agreement between the original intensity data and intensity data generated from each successive model. Refinement ends when the R-factor converges on a minimum wherein the model fits the electron density map and is stereochemically and conformationally reasonable. During refinement, ordered solvent molecules are added to the structure.

The present invention provides high-resolution three-dimensional structures and atomic structure coordinates of a crystalline XPA-ERCC1 complex. The present invention also identifies the XPA binding site of ERCC1, and consequently, amino acids of XPA and ERCC1 that participate in binding. The specific methods used to obtain the structure coordinates are provided in the examples, infra. The atomic structure coordinates of a crystalline XPA-ERCC1 complex, obtained from 4.0 Å resolution diffraction data and NMR distance measurements, are listed in Table 2.

Those of skill in the art will understand that a set of structure coordinates for a protein or a protein/ligand complex or a portion thereof, is a relative set of points that define a shape in three dimensions. Thus, it is possible that an entirely different set of coordinates could define a similar or identical shape. Moreover, slight variations in the individual coordinates will have little effect on the overall shape. These variations in coordinates may be generated because of mathematical manipulations of the XPA-ERCC1 structure coordinates. For example, the sets of structure coordinates shown in Table 2 could be manipulated by crystallographic permutations of the structure coordinates, fractionalization of the structure coordinates, application of a rotation matrix, integer additions or subtractions to sets of the structure coordinates, inversion of the structure coordinates or any combination of the above. Alternatively, modifications in the crystal structure due to mutations, additions, substitutions, and/or deletions of amino acids or other changes in any of the components that make up the crystal could also account for variations in structure coordinates. If such variations are within an acceptable standard error as compared to the original coordinates, the resulting three-dimensional shape should be considered to be the same. Thus, for example, a ligand that bound to the XPA binding pocket of ERCC1 would also be expected to bind to another binding pocket whose structure coordinates defined a shape that fell within the acceptable error.

According to the invention, a crystalline XPA-ERCC1 complex and the three-dimensional structural information derived therefrom can be used in structure based drug design. Structure based drug design refers to the use of computer simulation to predict a conformation of a peptide, polypeptide, protein, or conformational interaction between a peptide or polypeptide, and a therapeutic compound. For example, generally, for a protein to effectively interact with a therapeutic compound, it is necessary that the three dimensional structure of the therapeutic compound assume a compatible conformation that allows the compound to bind to the protein in such a manner that a desired result is obtained upon binding. Knowledge of the three dimensional structure of the complex, and particularly the structural coordinates of amino acids of a ligand and its binding site enables a skilled artisan to design a therapeutic compound having such a compatible conformation.

For example, knowledge of the three dimensional structure of the ERCC1 binding site of XPA peptide provides the basis to design a therapeutic compound that binds to ERCC1 or to XPA and results in inhibition of a biological response, such as formation of an XPA-ERCC1 complex and NER activity. Suitable structures and models useful for structure based drug design are disclosed herein. Preferred structures to use in a method of structure based drug design include the XPA binding site of ERCC1 protein, the XPA ligand as bound to ERCC1, and the binding region of an XPA-ERCC1 complex. One suitable model includes the coordinates of an ERCC1 complex as disclosed in Table 2. Another suitable model is deposited as 2A1I in the protein data bank. 2A1I provides atomic coordinates of the central domain of ERCC1 at about 2 Å resolution, and is useful in conjunction with information regarding the identity of the XPA binding site of the ERCC1 and the interaction of XPA with ERCC1, as provided herein.

The invention provides a method of determining the ability of a compound to inhibit the formation of an XPA-ERCC1 complex and/or inhibit NER activity. In certain embodiments, the method involves the use of cells, isolated proteins or protein fragments, and actual compounds. In other embodiments, inhibitors are identified in silico. In still other embodiments, both methods are used. The invention also provides a method for identifying inhibitors of XPA-ERCC1 interaction and NER activity by modifying known inhibitors of the complex. For example, a lead compound identified to be inhibitor is an in vitro or in vivo screen for inhibitors is docked with XPA-ERCC1 binding site residues and used as a basis for designing derivatives with improved pharmacological and/or pharmacokinetic properties. Accordingly, derivatives can be designed by replacing or substituting atoms or groups of atoms that, on the basis of the binding site model, are predicted to have improved binding and inhibition properties and/or to have improved bioavailability.

In one embodiment, the method comprises contacting a test compound with an ERCC1 polypeptide that binds to XPA and an XPA polypeptide of up to about 35 amino acids which comprises SEQ ID NO:1 from amino acid 70 to amino acid 78 under conditions in which a complex of the XPA and ERCC1 polypeptides can form in the absence of the compound, and measuring the binding of the ERCC1 polypeptide with the XPA polypeptide. A compound is identified as an inhibitor of complex formation when there is a decrease in the binding of the ERCC1 polypeptide with the XPA polypeptide in the presence but not the absence of the test compound. Examples of potential NER inhibitors include small molecules, peptides, and other polymeric molecules.

“Small molecule” refers to compounds that have a molecular weight up to about 2000 atomic mass units (Daltons). Any small molecule can be tested to determine whether it inhibits XPA-ERCC1 complex formation. In practice, small molecules to be tested are often compounds understood to have biological activity, which may be under development for pharmaceutical use. Generally such compounds will be organic molecules, which are typically from about 100 to 2000 Da, more preferably from about 100 to 1000 Da in molecular weight. Such compounds include peptides and derivatives thereof, steroids, anti-inflammatory drugs, anti-cancer agents, anti-bacterial or antiviral agents, neurological agents and the like. In principle, any compound under development in the field of pharmacy can be used in the present invention in order to facilitate its development or to allow further rational drug design to improve its properties. Libraries of high-purity small organic ligands and peptide agonists that have well-documented pharmacological activities are available from numerous sources, and can be screened directly or used in virtual screens.

The invention also provides peptide inhibitors of the XPA.ERCC1 complex. In an embodiment of the invention, the peptide contains the sequence Gly-Gly-Gly or Gly-Gly-Gly-Phe or Thr-Gly-Gly-Gly-Phe-Ile. In one embodiment, such a peptide is about 26 to about 35 amino acids in length. In another embodiment, the length is about 16 to about 25 amino acids. In another embodiment, the peptide is about 8 to about 15 amino acids in length. Certain peptide inhibitors of the invention are conformationally constrained to favor an ordered structure similar to that observed for amino acids 67 to 80 of XPA when bound to ERCC1. Such peptides include cyclic peptides and otherwise internally cross-linked peptides. In one embodiment, the constrained peptide includes the sequence Gly-Gly-Gly-Phe that is present in the ERCC1 binding loop of XPA. In another embodiment, the constrained peptide includes the sequence Thr-Gly-Gly-Gly-Phe-Ile. In one such embodiment, the binding loop of XPA is engineered to contain two cysteine residues, one on either side of the residues that participate in ERCC1 binding. The XPA binding loop is then constrained by the formation of a disulfide bond between the flanking cysteines. The cysteine residues need not be immediately adjacent to the XPA binding loop, but will usually be within a few amino acids. In an embodiment of the invention, the peptide is linked to a nuclear localization sequence.

NER inhibitors can also be found among “unnatural biopolymers” such as polymers consisting of chiral aminocarbonate monomers substituted with a variety of side chains. Cho et al, 1998, J. Am. Chem. Soc., 120, 7706-7718 discloses libraries of linear and cyclic oligocarbamate libraries and screening for binding to the integrin GPIIb/IIIa. Simon et al., 1992, Proc. Natl. Acad. Sci. 89, 9367-71 discloses a polymer consisting of N-substituted glycines (“peptoids”) with diverse side chains. Zuckermann et al., 1994, J. Med. Chem. 37, 2678-85 screened a library of such peptoids to obtain ligands with high affinity for the α₁-adrenergic receptor and the p-opiate receptor. Schumacher et al, 1996, Science 271, 1854-7 discloses D-peptide ligands specific for Src homology domain 3 (SH3 domain) by screening phage libraries of L-peptides against a proteins (SH3) synthesized with D-amino acids and then synthesizing a selected L-peptide using D-amino acids. Also included are aptamers (Jayasena, S. D., 1999, Clin. Chem. 45, 1628-50). All such compounds can be provided as libraries encompassing a large diversity of molecules. For example, Brody et al., 1999, Mol. Diagn. 4, 381-8 describes “how hundreds to thousands of aptamers can be made in an economically feasible fashion” and used in arrays.

Another embodiment of the invention is a method of identifying an agent that inhibits the formation of an XPA-ERCC1 complex and/or inhibits NER activity. The method generally includes determining which amino acid or amino acids of ERCC1 interact with XPA using a three dimensional model of a crystallized XPA-ERCC1 complex, and comparing the structural coordinates of the XPA-ERCC1 complex to the structure of a selected agent or using the structural coordinates of the XPA-ERCC1 complex to design an agent that interacts with ERCC1 or XPA at the those amino acids. Accordingly, the invention provides a method which comprises (a) providing structural coordinates defining all or a portion of the three-dimensional structure of the XPA binding site of ERCC1; (b) providing structural coordinates of the compound; and (c) fitting the structure of the compound to structural coordinates of the XPA binding site of ERCC1. In an embodiment of the invention, the structural coordinates of the XPA binding site of ERCC1 are provided in Table 2 or a portion thereof. For example, inspection of the XPA-ERCC1 crystal coordinates indicates that ERCC1 amino acids Arg106, Asn110, Phe 140, Ser142, Arg144, Tyr145, Leu148, H149, and Tyr152 form the pocket in which XPA binds and are close to or contact amino acids of XPA. Other amino acids around the periphery of the binding pocket that are buried in the complex include Asp129 and Arg156 of ERCC1. A compound that binds to ERCC1 and blocks binding of XPA need not bind to all of the amino acids of the XPA binding pocket. It is sufficient that the compound binds to ERCC1 and prevents XPA binding.

In another embodiment, the invention provides a method which comprises (a) providing structural coordinates defining all or a portion of the three-dimensional structure of the ERCC1 binding site of XPA; (b) providing structural coordinates of the compound; and (c) fitting the structure of the compound to structural coordinates of the XPA that interact with the XPA binding site of ERCC1. Inspection of the XPA-ERCC1 crystal coordinates indicates that XPA amino acids involved in ERCC1 binding include Gly72, Gly73, Gly74, Phe75, and that Thr71 and Ile76 are in close proximity to ERCC1.

In either embodiment, the fitting of step (c) may be assisted by computer, such as by computer modeling using a docking program. Docking may be accomplished by using software such as Quanta and Sybyl (manual model building software), followed by energy minimization and molecular dynamics with standard molecular mechanics force fields, such as CHARMM and AMBER. Specialized programs for docking include GRAM, GRID, Flexx, Glide, GOLD, MCSS, DOCK or AUTODOCK (See e.g. U.S. Pat. Nos. 5,856,116 and 6,087,478; Jorgensen W. L., 2004, Science 303, 1813-1818). Computer programs can be employed that estimate the attraction, repulsion, and steric hindrance of the compound with the XPA binding site of ERCC1.

The docking program may be connected to a structure generator (such as SYNOPSIS) to perform de novo screening. An alternative to de novo screening is creation of structures based on the binding site such as with programs including LUDI, SPROUT and BOMB, which allow a user to put a substituent in a binding site and then build up the substituent (Jorgensen W. L., 2004). Another alternative is to generate an idealized ligand. An idealized ligand can be created that is complementary to a binding site, filling in the void of the binding site, and providing locations of “probes” (e.g., hydrophobic surfaces, hydrogen bond acceptors corresponding to hydrogen bond donors of the binding site and vice versa). When a ligand exists that is aligned with a binding site, an idealized ligand can be created with probes based on features of the known ligand. Potential ligands from a database are fragmented, the fragments are aligned to the probes of the idealized ligand, and the fragments are chained together in a number of steps. At each step, van der Waals surface penetrations are minimized and hydrogen bond and hydrophobic surface interactions are improved.

In certain embodiments, identifying a test compound that binds to ERCC1 involves modifying a known compound such that it binds to one or more of Asn110, Asn129, Ser142, Arg144, Tyr145, Leu148, H149, and Tyr152 and fitting the modified compound to the structural coordinates of the XPA binding site of ERCC1. In another embodiment, the modified compound is fitted to structural coordinates of the ERCC1 binding domain of XPA, such as the coordinates of two or more of Thr71, Gly72, Gly73, Gly74, Phe75, and Ile76.

When screening, designing or modifying compounds, other factors to consider include the Lipinski rule-of-five, and Veber criteria, which are recognized in the pharmaceutical art and relate to properties and structural features that make molecules more or less drug-like.

One of skill in the art would appreciate that the above screening methods may also be carried out manually, by building an actual three dimensional model based on the coordinates, and then determining desirable antagonists based on that model visually.

As mentioned, a computer can be used to assist identification of NER inhibitors. Accordingly, the invention provides a computer-based method for the analysis of the interaction of a molecular structure with the XPA binding site of ERCC1. One embodiment of the invention provides a computer-assisted method for identifying a compound that inhibits NER activity using a processor, a data storage system, an input device, and an output device. The method of the invention comprises (a) inputting into the programmed computer through the input device data comprising the three-dimensional coordinates of all or a subset of the atoms of an XPA-ERCC1 complex as set out in Table 1; (b) providing a database of chemical and peptide structures stored in the computer data storage system; (c) selecting from the database, using computer methods, structures that meet certain physical or binding criteria; (d) and outputting to the output device the selected chemical structures. Optionally, NER inhibitory activity can be determined for the selected compounds.

The method may utilize the coordinates of atoms of interest of ERCC1 or XPA that are within 10-25 Å of selected amino acids involved in XPA-ERCC1 binding. These coordinates may be used to define a space, which is then analyzed in silico. Thus the invention provides a computer-based method for the analysis of molecular structures, which comprises providing the coordinates of at least two atoms of the XPA binding site of ERCC1, providing the structure of a compound to be fitted to the coordinates, and fitting the structure to the coordinates.

In practice, it will be desirable to model a sufficient number of atoms of the XPA-ERCC1 complex as defined by coordinates from Table 2, which represent the XPA-ERCC1 binding region. In an embodiment of the invention, there will be provided the coordinates of at least 5, at least 10, at least 50, or at least 100 selected atoms of the ERCC1 structure.

Although different compounds that bind to the XPA binding site of ERCC1 may interact with different parts of the binding pocket of the protein, the XPA-ERCC1 structure provided herein allows the identification of a number of particular sites which are likely to be involved in interactions with a compound that would inhibit XPA-ERCC1 complex formation.

As noted above for physically embodied compounds, molecular structures that may be tested using the coordinates provided will usually be compounds under development for pharmaceutical use. In principle, any compound under development in the field of pharmacy can be used in the present invention in order to facilitate its development or to allow further rational drug design to improve its properties.

As previously mentioned, libraries of high-purity small organic ligands and peptide agonists that have well-documented pharmacological activities are available from numerous sources. Usually, atomic coordinates of the compounds in the libraries are also available, and can be used in virtual screens. One example is an NCI diversity set which contains 1,866 drug-like compounds (small, intermediate hydrophobicity). Another is an Institute of Chemistry and Cell Biology (ICCB; maintained by Harvard Medical School) set of known bioactives (467 compounds), which includes many extended, flexible compounds. Some other examples of the ICCB libraries are: Chem Bridge DiverSet E (16,320 compounds); Bionet 1 (4,800 compounds); CEREP (4,800 compounds); Maybridge 1 (8,800 compounds); Maybridge 2 (704 compounds); Maybridge HitFinder (14,379 compounds); Peakdale 1 (2,816 compounds); Peakdale 2 (352 compounds); ChemDiv Combilab and International (28,864 compounds); Mixed Commercial Plate 1 (352 compounds); Mixed Commercial Plate 2 (320 compounds); Mixed Commercial Plate 3 (251 compounds); Mixed Commercial Plate 4 (331 compounds); ChemBridge Microformat (50,000 compounds); Commercial Diversity Set1 (5,056 compounds). Other NCI Collections are: Structural Diversity Set, version 2 (1,900 compounds); Mechanistic Diversity Set (879 compounds); Open Collection 1 (90,000 compounds); Open Collection 2 (10,240 compounds); Known Bioactives Collections: NINDS Custom Collection (1,040 compounds); ICCB Bioactives 1 (489 compounds); SpecPlus Collection (960 compounds); ICCB Discretes Collections. The following ICCB compounds were collected individually from chemists at the ICCB, Harvard, and other collaborating institutions: ICCB1 (190 compounds); ICCB2 (352 compounds); ICCB3 (352 compounds); ICCB4 (352 compounds). Natural Product Extracts: NCI Marine Extracts (352 wells); Organic fractions—NCI Plant and Fungal Extracts (1,408 wells); Philippines Plant Extracts 1 (200 wells); ICCB-ICG Diversity Oriented Synthesis (DOS) Collections; DDS1 (DOS Diversity Set) (9600 wells). Compound libraries are also available from commercial suppliers, such as ActiMol, Albany Molecular, Bachem, Sigma-Aldrich, TimTec, and others.

As mentioned, a compound obtained through rational design or identified by in silico methods can optionally be synthesized, and the ability of the compound to inhibit XPA mediated endonuclease activity of ERCC1-XPF measured.

One way to determine inhibition of NER activity is by determining the level of characteristic NER excision products in the presence or absence of increasing concentrations of a test compound. For example, as exemplified below, the excision of an oligonucleotide containing damage from a plasmid in a cell-free NER-proficient cell extract and the effects of a putative NER inhibitor on this reaction can be measured by radioactive labeling and analysis by denaturing PAGE. Inhibition of formation of an XPA-ERCC1 complex can be determined by, for example, by a competitive binding assay. In one such assay, a subsaturating amount of a fluorescein-labeled XPA₆₇₋₈₀ peptide is bound to the ERCC1₉₄₋₂₁₄ receptor. Binding of a competitor is detected as a loss of fluorescence polarization. Fluorescence polarization detection is centered on the principle that smaller molecules rotate faster than larger molecules in solution. Accordingly, rotation of XPA₆₇₋₈₀ is reduced by binding to ERCC1. Fluorescence can be used to probe these differences in rotation rates since the time required for a fluorophore to emit a photon after excitation requires a measurable amount of time, typically in the nanosecond range for most fluorophores. Rotation of fluorophore in that time period results in depolarization of the fluorescence emission. Thus differences in rotation between bound and unbound labeled ligand can be measured.

The invention also encompasses machine readable media embedded with the three-dimensional structure of the crystal complex described herein, or with portions thereof. As used herein, “machine readable medium” refers to any medium that can be read and accessed directly by a computer or scanner. Such media include, but are not limited to: magnetic storage media, such as floppy discs, hard disc storage medium and magnetic tape; optical storage media such as optical discs or CD-ROM; electrical storage media such as RAM or ROM; and hybrids of these categories such as magnetic/optical storage media. Such media further include paper on which is recorded a representation of the atomic structure coordinates, e.g., Cartesian coordinates, that can be read by a scanning device and converted into a three-dimensional structure with an OCR.

A variety of data storage structures are available to a skilled artisan for creating a computer readable medium having recorded thereon the atomic structure coordinates of the invention or portions thereof and/or X-ray diffraction data. The choice of the data storage structure will generally be based on the means chosen to access the stored information. In addition, a variety of data processor programs and formats can be used to store the sequence and X-ray data information on a computer readable medium. Such formats include, but are not limited to, Protein Data Bank (“PDB”) format (Research Collaboratory for Structural Bioinformatics; www.rcsb.org/pdb/docs/format/pdbguide2.2/guide2.2 frame.html); Cambridge Crystallographic Data Centre format (www.ccdc.cam.ac.uk/support/csd_doc/volume3/z 323.html); Structure-data (“SD”) file format (MDL Information Systems, Inc.; Dalby et al., 1992, J. Chem. Inf. Comp. Sci. 32:244-255), and line-notation, e.g., as used in SMILES (Weininger, 1988, J. Chem. Inf. Comp. Sci. 28, 31-36). Methods of converting between various formats read by different computer software will be readily apparent to those of skill in the art, e.g., BABEL (v. 1.06, Walters & Stahl, ©1992, 1993, 1994; www.brunel.ac.uk/departments/chem/babel.htm.) All format representations of the crystal coordinates described herein, or portions thereof, are contemplated by the present invention. By providing computer readable medium having stored thereon the atomic coordinates of the crystal of the invention, one of skill in the art can routinely access the atomic coordinates of the invention, or portions thereof, and related information for use in modeling and structure based design programs, described in detail herein.

The invention also provides compounds identified by the methods described above. One embodiment of the present invention is an isolated compound, identified by the methods described above, which is capable of inhibiting NER activity. In one embodiment, the invention provides an isolated compound characterized by the ability to associate with a binding pocket of ERCC1 and inhibit NER activity. In one embodiment, the compound comprises atoms having locations that correspond to atoms of XPA that contact atoms in the binding pocket of ERCC1. Preferably, the compound comprises atoms having locations that correspond to atoms of amino acids Thr71, Gly72, Gly73, Gly74, Phe75, and Ile76 of XPA or a subset thereof, whereas amino acids which form the XPA binding pocket of ERCC1 include, but are not limited to Arg106, Asn110, Asn129, Ser142, Arg144, Tyr145, Leu148, Tyr152, and Arg156.

The compound may be provided as a therapeutic composition, and the present invention thus provides therapeutic compositions comprising one or more compounds identified by methods described above. One embodiment of the present invention is a therapeutic composition that is capable of inhibiting NER activity.

In another embodiment of the invention, a compound identified by the methods described above is used to inhibit NER activity. Thus, the invention also provides a method of inhibiting NER activity comprising administering a therapeutically effective amount of a compound of the present invention. Another embodiment of the invention is a method of inhibiting growth of neoplasms, cancers or tumors in a mammal comprising administering a therapeutically effective amount of the compound of the present invention. A therapeutically effective amount is an amount that achieves the desired therapeutic result.

The compounds inhibit NER activity and can be used to treat or inhibit growth of, for example, testicular cancer, ovarian cancer, breast cancer, prostate cancer, cervical cancer, cancers of the head and neck, esophageal cancer, colorectal cancer, non-small cell lung cancer, pancreatic cancer, lymphoma, brain tumors such as glioblastomas, and the like.

Treatable tumors include primary and secondary, or metastatic, tumors. The compounds can also be used to treat refractory tumors. Refractory tumors include tumors that fail or are resistant to treatment with chemotherapeutic agents alone, radiation alone or combinations thereof. The NER inhibitory compounds are also useful to inhibit growth of recurring tumors, e.g., tumors that appear to be inhibited by treatment with chemotherapeutic agents and/or radiation but recur up to five years, sometimes up to ten years or longer after treatment is discontinued.

The compounds are particularly useful when administered with other chemotherapeutic agents that cause lesions in DNA, such as, for example, platinating agents. The combination may provide increased, additive, or synergistic effect. Further, many cancers eventually become resistant to such chemotherapeutic agents, usually by upregulation of DNA repair mechanisms such as NER. Accordingly, the compounds of the invention are used, not only to enhance the effect of chemotherapeutic agents, but also to overcome resistance associated with DNA repair mechanisms.

In another embodiment, the invention provides a method of treating a disease or condition in a mammal by administering a therapeutically effective amount of a compound of the present invention. While not intending to be bound by any particular mechanism, the diseases and conditions that may be treated by the present method include, for example, those in which DNA repair is undesirable. In one embodiment, the disease is a neoplastic disease. In another embodiment, the disease is cancer.

In another embodiment, the disease is a hyperproliferative disease. As used herein, “hyperproliferative disease” refers to a condition caused by excessive growth of non-cancer cells. An example of hyperproliferative disease is psoriasis. Psoriasis is a non-contagious skin disorder that most commonly appears as inflamed swollen skin lesions covered with silvery white scale. Other non-limiting examples of hyperproliferative disease include psoriasis, actinic keratoses, seborrkeic keratoses, acanthosis, scleroderma, and warts.

The compounds of the invention may be administered in combination with other therapeutic agents. In view of their NER inhibitory activity, compounds of the invention are usually administered with an agent that induces or enhances DNA damage. Further, because cancer cells often develop resistance to DNA damaging agents through induction of DNA repair pathways, the compounds of the invention are useful, not only for enhancing the effectiveness of DNA damaging agents, but also for prolonging their effectiveness.

Platinum-based chemotherapeutic agents cause DNA adducts that distort the three dimensional structure of the DNA double helix. Platinum agents, including cisplatin, carboplatin, and oxaliplatin, have been used clinically for nearly thirty years as part of the treatment of many types of cancers, including head and neck, testicular, ovarian, cervical, lung, colorectal, and lymphoma. Cisplatin (cis-Diaminodichloroplatinum or cis-DDP) is a neutral, square planar complex of platinum(II) that is coordinated to two relatively inert ammonia groups and two labile chloride ligands in cis geometry. Administered intravenously, cisplatin remains stable in the blood plasma until it diffuses into the cytoplasm of cells, where low salt (chloride) concentration leads to the substitution of the labile chloride ligands by water or hydroxide ions, yielding a charged and activated electrophilic agent. Subsequent reaction with nucleophilic sites on DNA results in the formation of monoadducts, intrastrand, or interstrand cross-links. Platination of oligonucleotides preferentially yields intrastrand N7-N7 cross-links between neighboring pyrimidine residues: 1,2-d(GpG) (accounting for up to 65% of all cisplatin-induced lesions) or 1,2-d(ApG) intrastrand cross-links (25% of all adducts) between adjacent bases, and intrastrand 1,3-d(GpNpG) adducts (5-10%) with one nucleotide (N) separating the cross-linked guanines. Another platinating agent is satraplatin, which is an orally available platinating agent. Platinum agents include analogs or derivatives of any of the foregoing representative compounds.

DNA alkylating agents are another general class of agents, and include the haloethylnitrosoureas, especially the chloroethylnitrosoureas. Representative members of this broad class include carmustine, chlorozotocin, fotemustine, lomustine, nimustine, ranimustine and streptozotocin.

General classes of compounds that are used for treating many cancers and that can be used with compounds of the invention include DNA alkylating agents and DNA intercalating agents. Some non-limiting examples of DNA alkylating agents are melphalan, and dacarbazine. Antibiotics that can alkylate or intercalate into DNA include amsacrine; actinomycin A, C, D (alternatively known as dactinomycin) or F (alternatively KS4); azaserine; bleomycin; caminomycin (carubicin), daunomycin (daunorubicin), or 14-hydroxydaunomycin (adriamycin or doxorubicin); mitomycin A, B or C; mitoxantrone; plicamycin (mithramycin); and the like. Psoralens are examples of light activable compounds that intercalate into and in combination with UV irradiation, induce cross-links in DNA. Psoralens are typically used in the photochemotherapeutic treatment of cutaneous diseases such as psoriasis, vitiligo, fungal infections and cutaneous T cell lymphoma. Useful anti-neoplastic agents also include mitotic inhibitors, such as taxanes docetaxel and paclitaxel.

Topoisomerase inhibitors are another class of anti-neoplastic agents that can be used in combination with compounds of the invention. These include inhibitors of topoisomerase I or topoisomerase II. Topoisomerase I inhibitors include irinotecan (CPT-11), aminocamptothecin, camptothecin, DX-8951f, topotecan. Topoisomerase II inhibitors include etoposide (VP-16), and teniposide (VM-26).

It is well established that radiation in the UV range is damaging to DNA. The UV spectrum is subdivided in three wavelength ranges: UVA (320-400 nm), UVB (290-320 nm), and UVC (200-290 nm). The formation of DNA photoproducts in human skin is maximal upon exposure up to 300 nm UV light, which correlates with the optimal absorption spectrum of thymine and cytosine.

Compounds of the invention can also be administered in treatments employing ionizing radiation. When the anti-neoplastic agent is radiation, the source of the radiation can be either external (external beam radiation therapy—EBRT) or internal (brachytherapy—BT) to the patient being treated.

Compounds of the invention can also be used in combination with treatments employing multiple agents. For example, an anti-metabolite, such as capecitabine (which is metabolized to 5-fluorouracil and inhibits pyrimidine synthesis) is often combined with a compound that forms DNA adducts, such as oxaliplatin. The compounds of the invention can be used to enhance such combinations of agents.

To provide for entry of inhibitory peptides into the nucleus of a cell, the peptides can be synthesized or expressed as fusions with a nuclear localization signal (NLS), which mediates macromolecular translocation into the nucleus. Two major classes of NLS are most commonly used for enhancing DNA transfection efficiency: 1) the classical NLS, which was originally found in SV40-T antigen and in nucleoplasmin, and which consists of a cluster of basic residues preceded by a proline residue, and 2) the non-classical M9 NLS of heterogeneous nuclear ribonucleoprotein (hnRNA) A1, which contains numerous glycine residues instead of the basic residues. The classical NLS binds directly to importin, a heterodimeric transport protein, which docks at the cytoplasmic face of the nuclear pore complex in an energy-dependent manner. The M9 NLS requires a different receptor for the nuclear translocation known as transportin. One way to administer such hybrid peptide inhibitors of NER is using liposomes. Another is to express the hybrid peptide from a gene therapy vector.

Modes of administration can include, but are not limited to, subcutaneous, intradermal, intravenous, intranasal, oral, transdermal, intraocular and intramuscular routes.

The dosage administered depends on numerous factors, including, for example, the type of agent, the type and severity tumor being treated and the route of administration of the agent. It should be emphasized, however, that the present invention is not limited to any particular dose.

In another aspect, the present invention provides pharmaceutically acceptable compositions that comprise a therapeutically-effective amount of one or more of the compounds of the present invention, including but not limited to the compounds described above and those shown in the Figures, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment, e.g. reasonable side effects applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals with toxicity, irritation, allergic response, or other problems or complications, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al., 1977, J. Pharm. Sci. 66, 1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, oral, intravenous, intracerebroventricular and subcutaneous doses of the compounds of this invention for a patient, when used for the indicated analgesic effects, will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms. Preferred dosing is one administration per day.

While it is possible for a compound of the present invention to be administered alone, it is preferable to administer the compound as a pharmaceutical formulation (composition).

The compounds according to the invention may be formulated for administration in any convenient way for use in human or veterinary medicine, by analogy with other pharmaceuticals.

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin, lungs, or mucous membranes; or (4) intravaginally or intrarectally, for example, as a pessary, cream or foam; (5) sublingually or buccally; (6) ocularly; (7) transdermally; or (8) nasally.

### Examples

The invention now being generally described, it will be more readily understood by reference to the following examples, which are included merely for purposes of illustration of certain aspects and embodiments of the present invention, and are not intended to limit the invention.

Peptide and DNA. The XPA₆₇₋₈₀ peptide corresponding to residues 67-80 of the XPA protein and the mutant peptide XPA₆₇₋₈₀^{F75A} were synthesized by solid phase methods then HPLC-purified by the Molecular Biology Core Facility at Tufts University (Boston, Mass.). A 40mer DNA oligomer 5′-CCGGTGGCCAGCGCTCGGCG(T)₂₀-3′ with a 5′ 6FAM label (Integrated DNA Technologies) was gel-purified by conventional techniques.

Protein expression and purification. The central domain of ERCC1 (constructs ERCC1₉₂₋₂₁₄ or ERCC1₉₆₋₂₁₄) with an N-terminal His₆ tag was expressed and purified as previously described (Tsodikov et al., 2005). Fragments of the XPA protein (XPA₁₋₂₇₃, XPA₅₉₋₂₇₃, XPA₅₉₋₂₁₉, XPA₅₉₋₉₃) were cloned into pET19 bpps, in which an N-terminal (His)₁₀ tag is separated from the XPA sequences by a Prescission protease cleavage site (gift of Dr. Tapan Biswas), between NdeI and XhoI sites. Full length XPA protein was expressed in bacteria from pET15b-XPA and purified by Ni²⁺-NTA, gel filtration, and 18 heparin chromatography.

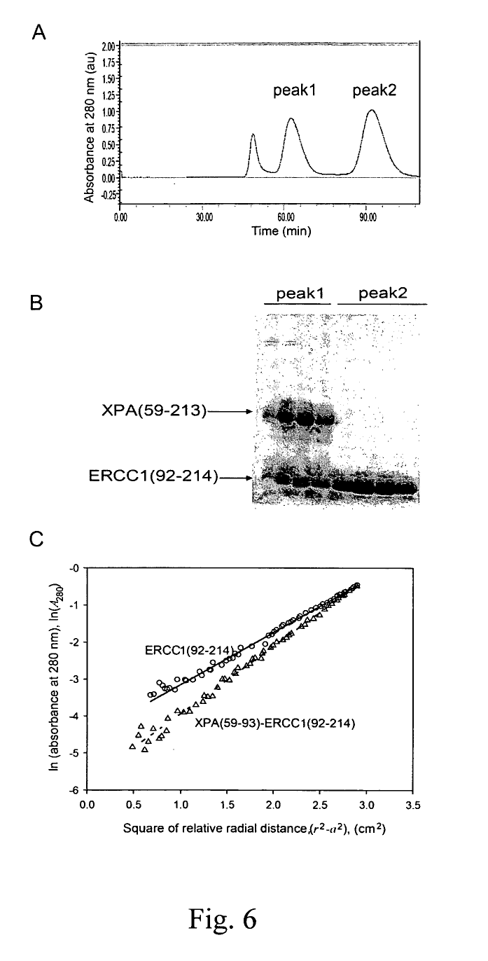

All proteins were expressed in BL21(DE3) E. coli (Stratagene). The cells were grown to OD₆₀₀=0.5 at 37° C., then cooled down to 22° C. and induced with 0.5 mM of IPTG at 22° C. for 15 hours. XPA proteins were purified by Ni²⁺ chromatography using a HiTrap Ni²⁺-chelating column (Amersham-Pharmacia) following manufacturer's instructions. These proteins was dialysed overnight and the His-tag was concomitantly cleaved in a buffer containing 30 mM Tris pH 8.0, 400 mM NaCl, 2 mM beta-mercaptoethanol and Prescission protease in approximately a 1:100 molar ratio to XPA. The XPA and ERCC1 proteins were further purified individually on S100 HiPrep (Amersham-Pharmacia) column. In order to obtain homogeneous XPA/ERCC1 complexes, purified XPA and ERCC1 were combined in excess of ERCC1 (for XPA₁₋₂₇₃, XPA₅₉₋₂₇₃, XPA₅₉₋₂₁₉) or of XPA (for XPA₅₉₋₉₃ and XPA₆₇₋₈₀) and the same gel filtration purification step was repeated. The peak corresponding to XPA-ERCC1 was well separated from the excess ERCC1 or XPA for all XPA constructs. This separation or the shapes of the peaks was not affected in the salt concentration range of 50-400 mM NaCl. For the XPA₅₉₋₂₁₉ fragment containing the Zn²⁺-binding domain of XPA (FIGS. 6A&B), an elemental analysis performed on the corresponding XPA-ERCC1 complex indicated that 98% of XPA₅₉₋₂₁₉ contains a Zn²⁺ atom. It was concluded that the structured part of XPA remained properly folded in association with ERCC1.

Labeled proteins for NMR studies were produced in M9 minimal media containing ¹⁵N-labeled NH₄Cl and ¹³C-labeled glucose as the sole sources of nitrogen and carbon, respectively. A perdeuterated, ¹⁵N-labeled ERCC1 sample was prepared in media containing 100% D₂O with 100% deuterated glucose and ¹⁵N-labeled NH₄Cl.

Analytical ultracentrifugation. Sedimentation equilibrium experiments with ERCC1₉₂₋₂₁₄ and the complex ERCC1₉₂₋₂₁₄-XPA₅₉₋₉₃ were performed using Beckman XLA Analytical Centrifuge. In both cases, proteins were at concentrations of 0.3-0.5 mg/ml in NMR Buffer (20 mM Tris buffer pH 7.2, 50 mM NaCl, 2 mM β-mercaptoethanol and 0.1 mM EDTA). Sedimentation equilibrium data were analyzed as follows:

Absorbance was analyzed using equation (1):

$${A}_{280} ( r ) = {A}_{280} ( a ) exp ( \frac{{ω}^{2} M ( 1 - ρ v ) ( {r}^{2} - {a}^{2} )}{2 RT} ) \qquad ( 1 )$$

in which A₂₈₀ is the absorbance at 280 nm, r and a are an arbitrary and a reference radial distances, ω=2πf (where f=40,000 min⁻¹) is the angular velocity of the rotor, ρ=1 g/mL is the density of water, M is the molecular weight of the sedimented species, R is the Boltzmann constant and T=277 K is the absolute temperature.

The nonlinear regression fitting of the data to Equation (1) to determine M was performed using SigmaPlot 9.0 (SSP). Data were fit (FIG. 6c; solid and dashed lines) using a single-component, non-interacting model and assuming partial specific volumes v=0.75 mL/g for both samples. The fit yields molecular weights of (15.0±1.0) kDa and (19.4±1.2) kDa for free ERCC1₉₂₋₂₁₄ and ERCC1₉₂₋₂₁₄-XPA₅₉₋₉₃ complex respectively.

Protein crystallization and data collection. The complex of ERCC1₉₆₋₂₁₄-XPA₆₇₋₈₀ was concentrated to 9 mg/ml using Amicon (Millipore) concentrator with a 5 kDa molecular weight cut-off in 30 mM Tris pH 8.0, 200 mM NaCl, 2 mM beta-mercaptoethanol and 0.1 mM EDTA. Crystals were grown by vapor diffusion in hanging drops containing of the protein solution and 1 μL of the reservoir solution (100 mM Tris pH 8.5, 2 M ammonium dihydrogen phosphate, 10% glycerol) at 21.5° C. Single cubic crystals of XPA-ERCC1 complex grew in 1-2 weeks, reaching a size of 0.15-0.20 mm in each dimension. The crystals diffracted to 4.0 Å resolution using a rotating anode X-ray source at Harvard-Armenise X-ray facility. I/σ(I) decreases sharply (R_{merge} increases sharply) with increasing resolution at 4 Å. As a result, higher resolution shells contain no data useful for structure refinement.

A complete and redundant x-ray data set was collected and processed using HKL2000 (Otwinowski, 1997, Methods Enzymol., 276, 307-326). The crystals belong to space group I4₁32 with one ERCC1-XPA complex in the asymmetric unit. The structure was determined by molecular replacement (MR) methods using the program PHASER (McCoy et al., 2005, Acta Crystallogr. D Biol. Crystallogr., 61, 458-464) and the crystallographic model of the ERCC1 central domain (Tsodikov et al., 2005; PDB code 2A1I) in which the residues C-terminal to residue 214 were deleted. A difference (Fo−Fc) electron density map calculated with phases from the MR solution revealed the bound XPA peptide. The XPA peptide was built into the difference density using distance restraint information from NMR experiments and the structure of the complex was then refined as described below, with strong geometric restraints imposed on the ERCC1 subunit due to the low resolution diffraction data and the absence of intramolecular distance information for ERCC1. All experimental XPA-ERCC1 distance restraints were accommodated without violations using the structure of unbound ERCC1 suggesting that ERCC1 does not undergo significant conformational changes upon binding XPA.

NMR experiments and determination of the structure of XPA-ERCC1 complex. All NMR data were acquired in the NMR Buffer described above. The protein concentrations were 0.25 mM for free ERCC1₉₆₋₂₁₄ or ERCC1₉₂₋₂₁₄ (which behaved similarly in all experiments), 0.25 mM for ERCC1₉₂₋₂₁₄ in complex with a synthetic XPA₆₇₋₈₀ peptide and 0.1 mM for ERCC1₉₂₋₂₁₄ complex with XPA₅₉₋₉₃ fragment. Higher protein concentrations resulted in line broadening and lower quality NMR spectra. Backbone assignments of the free ERCC1₉₂₋₂₁₄ and ERCC1-XPA₆₇₋₈₀ complex were performed using a standard set of triple-resonance experiments: HNCA/HN(CO)CA, HN(CA)CB/HN(COCA)CB and HNCO/HN(CA)CO.

Structural information for the ERCC1-XPA complex was obtained with a differentially-labeled sample in which ERCC1 was ¹⁵N-labeled and perdeuterated and the synthetic XPA fragment was unlabeled (D,N-ERCC1/U-XPA) (Walters et al., 2001, Methods Enzymol., 339, 238-258; Walters, 1997, J. Am. Chem. Soc., 119, 5958-5959). The assignment of the XPA peptide in this sample was performed using homonuclear 2D NOESY and 2D TOCSY experiments acquired in both H₂O and D₂O buffers. The total of 92 intramolecular distance constraints for the XPA peptide were derived from the 2D NOESY experiment acquired in H₂O with 100 msec mixing time. Intermolecular distance restraints were derived from a ¹⁵N-dispersed NOE-HSQC experiment acquired on the D,¹⁵N-ERCC1/U-XPA sample using 200 msec mixing time. A total of 23 intermolecular distance restraints between the amide protons of ERCC1 and the protons of XPA were derived from this experiment. The structure of the ERCC1-XPA complex was calculated using simulated annealing procedure in XPLOR-NIH (Schwieters et al., 2003, J. Magn. Reson., 160, 65-73). The total energy term used in the calculation incorporated all of the NMR-derived distance restraints as well as the 4 Å X-ray data. Ten lowest energy structures out of 100 calculated were deposited in the PDB with accession code 2JNW. The solvent accessible surface areas were calculated for the lowest energy structure using Surface Racer 4.0 (Tsodikov et al., 2002) with the solvent probe radius of 1.4 Å.

TABLE 1
Data collection and NMR and X-ray structure determination statistics.
X-ray diffraction and refinement without using NMR data

| | |
|---|---|
| Space group | I4₁32 |
| Number of XPA-ERCC1 complexes per a.u. | 1 |
| Resolution (highest resolution shell) | 40-4.1 Å (4.3-4.1 Å) |
| I/σ | 16.8 (6.0) |
| Redundancy | 10.5 (10.5) |
| R_{merge} | 0.15 (0.41) |
| Number of unique reflections | 1538 |
| R/R_{free} without XPA, prior to | 0.33/0.38 |
| refinement using NMR data | |

NMR and refinement using X-ray data

| | |
|---|---|
| Total NOE Distance Restraints | 109 |
| Intermolecular (ERCC1-XPA) | 18 |
| Intramolecular (XPA) | 91 |
| Intraresidue | 61 |
| Interresidue | 30 |
| Hydrogen Bond Restraints | 1 |
| Dihedral Angle Restraints | 0 |
| <RMSD> from mean structure (XPA 70-77) | 0.19/0.44 |
| backbone/heavy atom (Å) | |

Ramachandran Plot (% residues)

| | |
|---|---|
| Most Favorable Region | 77.6 |
| Additionally Allowed Region | 18.7 |
| Generously Allowed Region | 3.7 |
| Disallowed Region | 0 |

Construction and expression of mutant XPA proteins. Site-directed mutagenesis using the QuikChange kit (Stratagene) introduced point mutations in the expression vector pET15b-XPA. pET15b-XPA served as template and oligonucleotide primers used to generate the mutations contained the desired mutation and a marker restriction site for selection. The following primers were used (Xba I restriction site are underlined, modified nucleotides are shown in italics):

(SEQ ID NO: 4)
XPA-F75A: GACACAGGAGGAGGCGCCATTCTAGAAGAGGAAGAAG
(SEQ ID NO: 5)
XPA-ΔG74: GACACAGGAGGATTCATTCTAGAAGAGGAAGAAG
(SEQ ID NO: 6)
XPA-ΔG73/74: GATAATTGACACAGGATTCATTCTAGAAGAGGAAGAAG

Positive clones were fully sequenced to rule out the introduction of additional mutations. Mutant XPA proteins were expressed in E. coli BL21(DE3)pLyS cells and purified by chromatography on nickel-NTA, gel filtration, and heparin columns.

Nuclease assay. A substrate consisting of a 12 base pair stem with a 22 nucleotide loop (5′-GCCAGCGCTCGG(T)₂₂CCGAGCGCTGGC; SEQ ID NO:7) was 5′ end labeled using a T4 polynucleotide kinase and [γ-³²P]-ATP. The DNA substrate (100 fmol; at a final concentration of 6.7 nM) was suspended in nuclease buffer (25 mM Tris HCl pH 8.0, 40 mM NaCl, 10% glycerol, 0.5 mM β-mercaptoethanol, 0.1 mg/ml BSA) containing 0.4 mM MnCl₂ then incubated with ERCC1-XPF (100 or 400 fmol, corresponding to 6.7 nM or 26.8 nM) in the presence of 92 μM XPA₆₇₋₈₀ or XPA₆₇₋₈₀^{F75A}. The final reaction volume was 15 μl. These DNA cleavage reactions were incubated at 30° C. for 15 min then stopped by adding 10 μl of loading dye (90% formamide/10 mM EDTA) and heating at 95° C. for 5 min. Samples were analyzed by 15% denaturing PAGE (0.5×TBE) and the reaction products were visualized using a PhosphorImager (Typhoon 9400; Amersham Biosciences).

DNA binding assays. The three-way junction DNA substrate described previously (substrate 7 in Table 1 of Hohl et al., 2003, J. Biol. Chem., 278, 19500-19508) was 5′-³²P-end labeled and incubated at 1 nM concentration with various amounts of XPA in EMSA buffer (25 mM Hepes·KOH pH 8.0, 30 mM KCl, 10% glycerol, 1 mM DTT, 1 mM EDTA, 0.1 mg/ml BSA) at a reaction volume of 15 μl. After equilibration at room temperature for 30 min, the samples were loaded on a 5% (37.5:1) native polyacrylamide gel containing 0.5×TBE and electrophoresed at 90 V for 2 hrs. Gels were dried and the radioactive bands visualized by autoradiography.

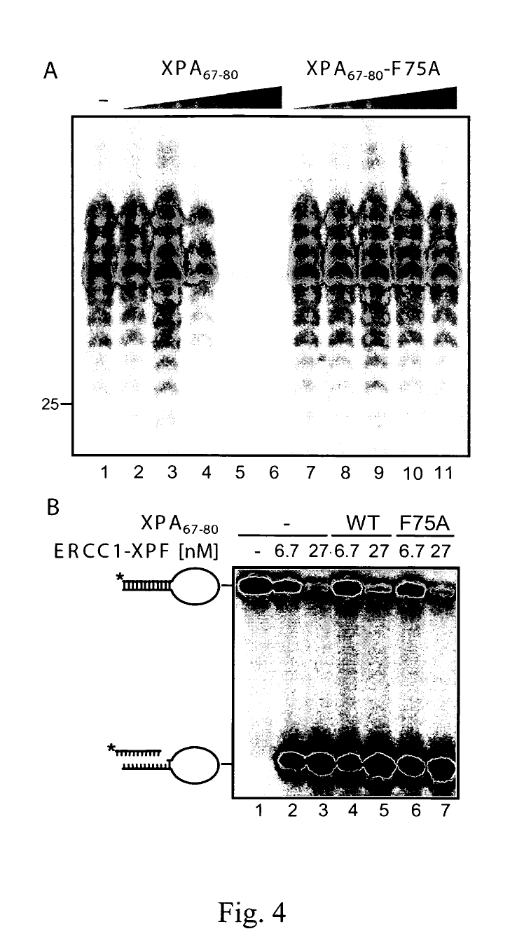

In Vitro NER assay. HeLa cell extracts and plasmid containing 1,3-intrastrand cisplatin adduct were prepared as described previously (Moggs et al., 1996, J. Biol. Chem. 271:7177; Shivji et al., 1999, Methods Mol. Bio. 113:373). Hela cell extract (2 μl) or XP-A (XP2OS) cell extract (3 μl), 2 μl of 5× repair buffer (200 mM Hepes-KOH, 25 mM MgCl₂, 110 Mm phosphocreatine (di-Tris salt, Sigma), 10 mM ATP, 2.5 mM DTT and 1.8 mg/ml BSA, adjusted to pH 7.8), 0.2 μl 2.5 mg/ml creatine phosphokinase (rabbit muscle CPK, Sigma) and either purified XPA peptide, XPA protein (WT, F75A, G73Δ or G73Δ/G74Δ) or NaCl (final NaCl concentration was 70 mM) in a total volume of 10 μl were preincubated at 30° C. for 10 min. One μL of a covalently-closed circular DNA plasmid (50 ng) containing the 1,3-intrastrand cisplatin crosslink was added before incubating the mixture at 30° C. for 45 min. After placing the samples on ice, 0.5 μl of 1 μM 35-mer oligonucleotide (5′-GGGGGAAGAGTGCACAGAAGAAGACCTGGTCGACCp-3′; (SEQ ID NO:8) was added and the mixtures heated at 95° C. for 5 min. The samples were allowed to cool down at room temperature for 15 min to allow the DNA to anneal. One μl of a Sequenase/[α-³²P]-dCTP mix (0.5 units of Sequenase and 2.5 μCi of [α-³²P]-dCTP per reaction) was added before incubating at 37° C. for 3 min, 1.2 μl of dNTP mix (100 μM of each dATP, dTTP, dGTP; 50 μM dCTP) was added and the mixture incubated for another 12 min. The reactions were stopped by adding 8 μl of loading dye (90% formamide/10 mM EDTA) and heating at 95° C. for 5 min. The samples were run on a 20% sequencing gel (0.5×TBE) at 45 W for 2.5 hrs. A low molecular weight DNA marker (New England Biolabs) was used as a reference after end-labeling the DNA with [α-³²P]-dCTP and Klenow fragment polymerase. The reactions products were visualized using a PhosphorImager (Typhoon 9400, Amersham Biosciences). Slight variation in band intensities (e.g. higher intensities in FIG. 4A, lane 3 in the main text) is due to experimental variability in the amount of material loaded in different lanes of the gel.

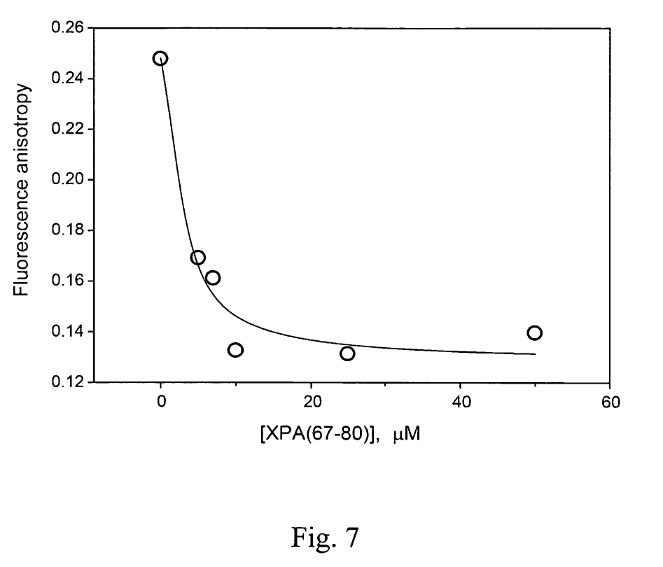

Competitive binding equilibrium titrations. The equilibrium titrations were performed in a binding buffer containing 20 mM Tris, pH 8.0, 20 mM NaCl, 2 mM betamercaptoethanol. The concentrations of single-stranded 5′ 6-FAM-labeled DNA (5′-CCG GTG GCC AGC GCT CGG CG(T)₂₀; SEQ ID NO:9) and ERCC1₉₆₋₂₁₄ were 50 nM and 2.33 μM, respectively, the concentration of the XPA peptide was varied from 0 to 30 μM (FIG. 7). Fluorescence anisotropy measurements were performed as previously reported (Tsodikov et al., 2005). Increasing XPA peptide concentration above 30 μM caused an increase in fluorescence anisotropy and quenching of fluorescence due to nonspecific interactions with the peptide, observed in the presence or absence of ERCC1 (data not shown).

The simplest binding model used in the data analysis consisted of two competitive equilibria, binding of XPA peptide and of the single-stranded 6FAM-labeled 40-mer DNA to the central domain of ERCC1. This model is consistent with the fact that at sufficiently high XPA concentration, the fluorescence anisotropy signal approaches that of unbound DNA. Therefore the two binding equilibria could be written as:

$$\begin{matrix}E + D {↔}^{ {K}_{DNA }} ED & ( 2 a ) \\ E + X {↔}^{ {K}_{XPA }} EX & ( 2 b )\end{matrix}$$

where E, D, X represent the ERCC1, DNA and XPA species, respectively. ED and EX represent ERCC1-DNA and ERCC1-XPA complexes, and K_{DNA} and K_{XPA} are the observed equilibrium association constants, defined in terms of the equilibrium concentrations of the species from Eqs. (2a) and (2b) as:

$$\begin{matrix}{K}_{DNA} = \frac{[ ED ]}{[ E ] [ D ]} & ( 3 a ) \\ {K}_{XPA} = \frac{[ EX ]}{[ E ] [ X ]} & ( 3 b )\end{matrix}$$

The total concentrations of reaction species according to conservation of material are represented as:

[X]ₜₒₜ=[X]+[EX]=[X]+K_{XPA}[E][X] (4a)

[E]ₜₒₜ=[E]+[EX]+[ED]≈[E]+K_{XPA}[E][X] (4b)

[D]ₜₒₜ=[D]+K_{DNA}[E][D] (4c)

The approximation made in Equation (4b) is due to the large excess of ERCC1 over DNA at the experimental conditions. It was assumed that each single-stranded 40-mer DNA oligonucleotide contains only one binding site for ERCC1. Because the central domain of ERCC1 occludes 10-15 nucleotides upon binding DNA (1), with a reasonable approximation, a maximum of 2-3 ERCC1 molecules could simultaneously bind to one DNA molecule without significant cooperativity. In this analysis, a value of (K_{DNA})₋₁=1.5 μM was used, obtained from the direct titration of ERCC1 at constant concentration (50 nM) of the same DNA oligomer, using the same single site approximation (1). Therefore the value of the equilibrium binding constant for XPA-ERCC1 complex formation, K_{XPA}, should be unaffected by the stoichiometry of ERCC1-DNA binding.

The system of Eqs. 4a, 4b and 4c yields expressions for [E], [X] and [D], which are then used to determined the fraction of bound DNA species, f, as follows:

$$\begin{matrix}[ X ] = \frac{\begin{matrix}{K}_{XPA} ( {[ X ]}_{tot} - {[ E ]}_{tot} - 1 + \\ \sqrt{{( {K}_{XPA} ( {[ X ]}_{tot} - {[ E ]}_{tot} ) - 1 )}^{2} + 4 {{K}_{XPA} [ X ]}_{tot}}\end{matrix}}{2 {K}_{XPA}} , & ( 5 ) \\ [ E ] = \frac{{[ E ]}_{tot}}{( 1 + {K}_{XPA} [ X ] )} , & ( 6 ) \\ [ D ] = \frac{{[ D ]}_{tot}}{( 1 + {K}_{DNA} [ E ]} , \\ and & ( 7 ) \\ f = \frac{{K}_{DNA} [ E ] [ D ]}{{[ D ]}_{tot}} & ( 8 )\end{matrix}$$

The observed fluorescence anisotropy, r, is then given by

r=r₀+(rₘₐₓ−r₀)f (9)

where r₀ and rₘₐₓ are fluorescent anisotropy values of unbound and fully bound DNA, respectively. SigmaPlot 9.0 was used to perform non-linear regression analysis of the data using Eq. 9 in order to obtain the best-fit value for K_{XPA}.

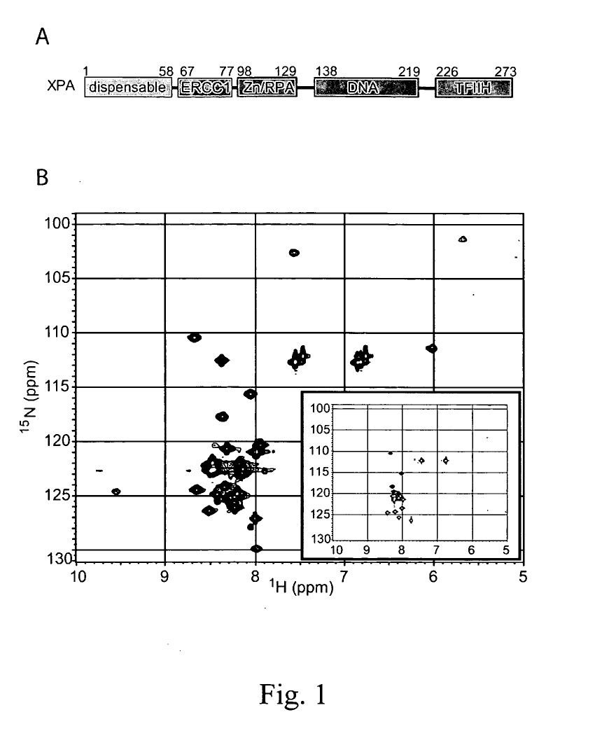

Induced fit of the XPA peptide upon interaction with ERCC1. Previous reports have suggested that the ERCC1-interacting region of XPA (FIG. 1A) is unfolded in solution, based on NMR studies and its sensitivity to proteolytic cleavage (Buchko et al., 2001; Iakoucheva et al., 2001). To investigate the structure of the XPA ligand, HSQC NMR spectra of a ¹⁵N-labeled XPA₅₉₋₉₃ peptide was collected, alone and in complex with unlabeled central domain of ERCC1 (the ERCC1₉₂₋₂₁₄ protein; FIG. 1B). In the absence of ERCC1, the resonance signals for XPA cluster in a narrow range of chemical shifts (FIG. 1B, inset) that is characteristic of an unstructured polypeptide with poor spectral dispersion. In the complex with ERCC1, a subset of XPA backbone amides become well-dispersed and the peaks are broader. These changes are indicative of a well structured region within the bound XPA peptide. Only a few resonance peaks are markedly perturbed when XPA₅₉₋₉₃ is bound to ERCC1, and among these, three glycine residues (assigned as Gly72, Gly73 and Gly74) are strongly perturbed in the complex. In order to overcome the peak broadening observed in NMR spectra of the XPA₅₉₋₉₃ peptide at concentrations above 0.1 mM, shorter XPA peptide ligands for ERCC1 were identified. A well behaved XPA₆₇₋₈₀ peptide (described below) was identified by expressing a series of XPA fragments that overlap with the previously identified region that binds to ERCC1 (Li et al., 1994).

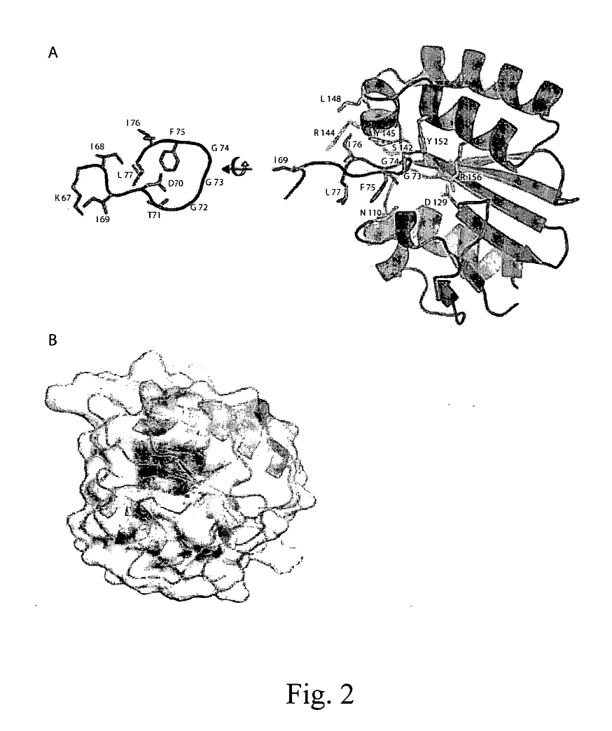

The structure of XPA in complex with ERCC1. A synthetic XPA₆₇₋₈₀ peptide with amino acid sequence KIIDTGGGFILEEE forms a stable complex with ERCC1₉₆₋₂₁₄ that can be purified by gel filtration chromatography. Like full length XPA protein, the XPA₅₉₋₉₃ and the XPA₆₇₋₈₀ peptides behave similarly and efficiently copurify with ERCC1, suggesting that XPA₆₇₋₈₀ contains all significant binding determinants. It was confirmed that XPA and ERCC1 form a stoichiometric 1:1 complex by estimating the amount of each subunit in the purified complex using an Edman degradation reaction, and by analytical centrifugation of the complex. Equilibrium sedimentation data for the complex (Supplementary FIG. 1C) were best fit to the expected masses for a 1:1 complex of XPA₅₉₋₉₃ and ERCC1₉₂₋₂₁₄ (Mw=(19.4±1.2) kDa) and unbound ERCC1₉₂₋₂₁₄ (Mw=(15.0±1.0) kDa). A structure of the XPA₆₇₋₈₀-ERCC1₉₆₋₂₁₄ complex (FIG. 2A) was determined by a combination of NMR-derived distance restraints and X-ray diffraction data extending to 4 Å resolution (Table 1) as described below.

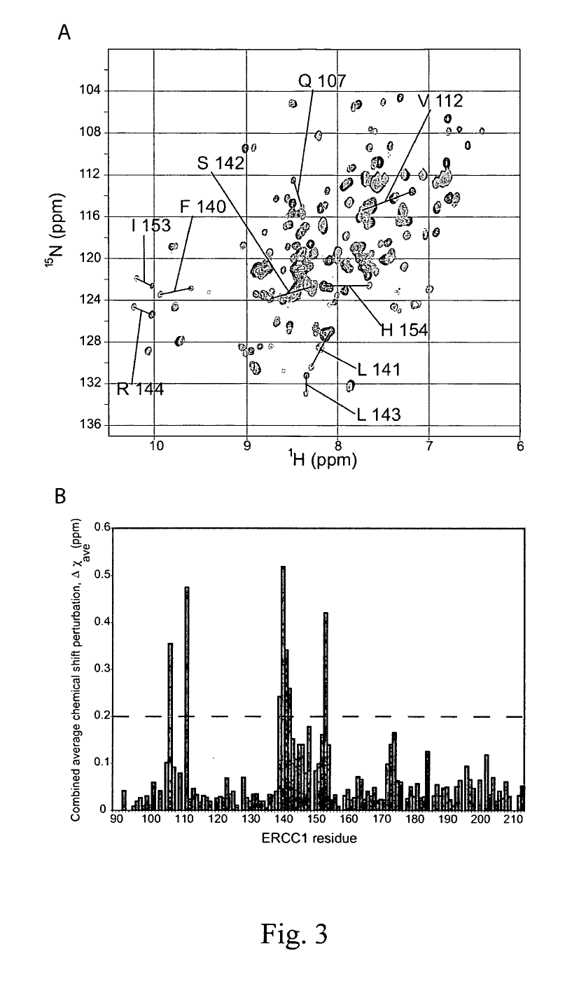

Identification of the ERCC1 binding site in complex with XPA. The binding site for XPA on the surface of ERCC1 (FIG. 2B) was identified using two dimensional HSQC experiments. The spectrum of unliganded ¹⁵N-labeled ERCC1₉₂₋₂₁₄ showed significant differences from that of the complex with unlabeled XPA₆₇₋₈₀ (FIG. 3). However, complexes of ERCC1₉₆₋₂₁₄ with either XPA₆₇₋₈₀ or XPA₅₉₋₉₃ were identical, suggesting that the shorter XPA peptide makes all of the significant binding contacts. The ¹⁵N HSQC spectrum of the ERCC1-XPA complex is consistent with a slow-exchange regime, implying a dissociation equilibrium constant below 1 μM for the complex. The XPA binding site on the ERCC1 central domain was identified using the backbone assignments for ERCC1₉₆₋₂₁₄ alone and in complex with XPA (see below).

A comparison of the ¹⁵N HSQC spectra for ERCC1 in the presence and absence of XPA reveals that, with only one exception, the most prominent changes in chemical shifts involve a cluster of residues within a V-shaped groove of the ERCC1 central domain (FIGS. 2B, 3). The bound XPA peptide fits snugly into the V-shaped groove of ERCC1 (FIG. 2). Three consecutive glycines (Gly72, Gly 3, Gly74) of the XPA peptide insert into the groove, making a U-turn with close steric complementary to the binding site. These are the same three conserved glycines previously reported to be essential for the interaction of XPA with ERCC1 and required for the functional complementation of XP-A cells (Li et al., 1994; Li et al., 1995). A total of 1039 Å² of accessible surface area from XPA peptide is buried in the complex with ERCC1, accounting for 61% of the solvent accessible surface area of XPA residues 67-77, which are in close proximity to the binding site. The XPA ligand derives many interactions from the core sequence motif (shown in boldface; KIIDTGGGFILEEE) of the XPA₆₇₋₈₀ peptide. The side chains of Phe75, Leu77 and Thr71 of XPA are clustered together at the mouth of the V-shaped groove (FIG. 2A) where Phe75 stacks against Asn110 of ERCC1, and the Ile76 side chain packs against the aliphatic portion of ERCC1 side chains Arg144 and Leu148. The binding groove in ERCC1 is capped by XPA Leu77. The glycine-rich loop of XPA₆₇₋₈₀ extends far into the groove of ERCC1 where main chain atoms of these XPA residues stack against the side chains of Tyr145 and Tyr152 from ERCC1 (FIG. 2A). The main chain amides of these glycines could participate in hydrogen bonding interactions with the ERCC1 binding site, although these interactions cannot be directly observed from our NMR experiments nor can they be reliably confirmed by low resolution (4 Å) X-ray diffraction data (Table 1). Based on the proximity of atoms modeled in the complex, it can be inferred that the carbonyl oxygen of Gly74 may bond with the main chain amide of Ser142 from ERCC1. The orientations of the Tyr145 and Tyr152 side chains from ERCC1 would permit their hydroxyl groups to make hydrogen bonding interactions with the backbone carbonyls of Thr71 and Gly73, respectively. The side chain of XPA Asp70 could participate in electrostatic interactions with the side chain His149 of ERCC1. It is notable that a solvent-exposed salt bridge between the side chains of Asp 129 and Arg156 of ERCC1 (PDB code 2A1I; (Tsodikov et al., 2005)) becomes almost completely buried when XPA is bound.

Phe75 of XPA is completely buried within the ERCC1 binding site (FIG. 2A). An alanine substitution at this position was tested for interference with binding to ¹⁵N-labeled ERCC1 by measuring chemical shifts in the 15N HSQC spectra in the presence of the mutant peptide designated XPA₆₇₋₈₀^{F75A}. Addition of the mutant peptide failed to perturb the chemical shifts of ERCC1 seen upon addition of wild-type XPA₆₇₋₈₀ (data not shown), indicating that the mutant peptide does not bind to ERCC1. The TGGGFI binding motif of the XPA ligand and the corresponding residues of the ERCC1 binding site are strictly conserved in higher eukaryotes. In lower eukaryotes, the corresponding sequences of both proteins have diverged from this consensus, perhaps indicating the coevolution of these two proteins and their functions.

The XPA peptide inhibits NER in mammalian cell extracts. The direct interaction of XPA₆₇₋₈₀ peptide with the ERCC1 binding pocket raised the possibility that this peptide might specifically interfere with the recruitment of the ERCC1-XPF nuclease into the NER reaction pathway. The effect of XPA₆₇₋₈₀ and the mutant XPA₆₇₋₈₀^{F75A} peptide on the dual incision of a DNA lesion during NER in cell free extracts was investigated. A plasmid containing a single site-specific 1,3-cisplatin intrastrand crosslink was incubated with HeLa cell free extract in the presence of increasing concentrations of XPA peptide (Shivji, 1999). In the absence of XPA peptide, the characteristic NER excision products of 28-33 nucleotides containing the lesion were evident (FIG. 4A, lane 1). Increasing concentrations of XPA₆₇₋₈₀ interfered with excision of the oligonucleotide, and complete inhibition was achieved at a concentration of XPA peptide in the low micromolar range (FIG. 4A, lanes 2-6). In contrast, the addition of XPA₆₇₋₈₀^{F75A} did not affect NER activity at concentrations up to the maximum concentration tested (FIG. 4A, lanes 7-11). The XPA peptide might inhibit NER activity in vitro by directly interfering with the endonuclease activity of ERCC1-XPF, instead of blocking its interaction with XPA. To account for the former possibility, the effect of XPA peptides on the DNA incision reaction catalyzed by purified ERCC1-XPF was tested using a stem-loop DNA substrate (de Laat et al., 1998, J. Biol. Chem., 273, 7835-7842). ERCC1-XPF efficiently cleaves on the 5′ side of the loop and the XPA peptide has no effect on this activity (FIG. 4B) even at a concentration (92 μM) that completely abolishes NER activity (FIG. 4A). It was concluded that the inhibitory effect of XPA₆₇₋₈₀ on the NER reaction results from disrupting the interaction of ERCC1 with XPA, an essential protein-protein interaction for the dual incision of DNA by the NER pathway.

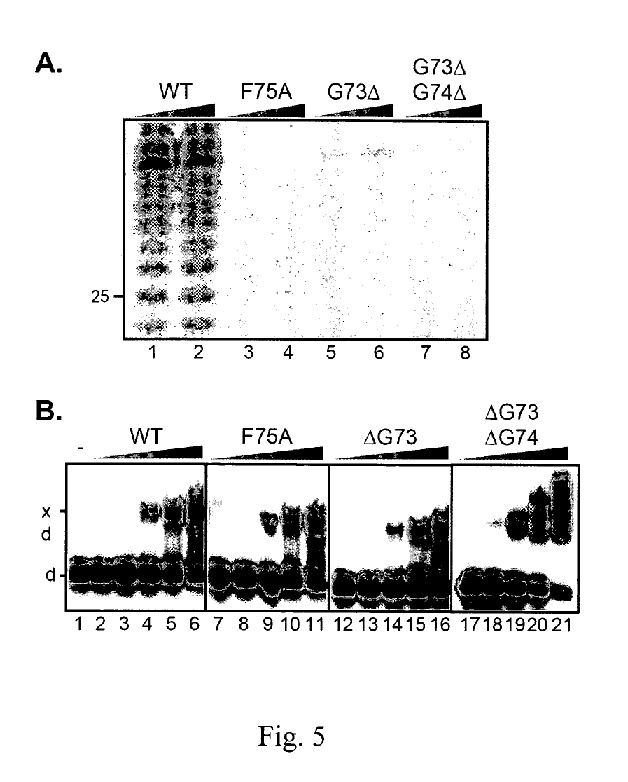

Mutations in the ERCC1 binding epitope of XPA abolish NER. The specificity of inhibition of NER by XPA₆₇₋₈₀ suggested that mutations of single residues such as F75 might diminish the NER activity of the XPA protein. Mutant XPA proteins were generated containing an F75A mutation and ΔG73 single and ΔG73/ΔG74 double deletion, and compared their activities to that of the wild-type XPA protein. The ability of the XPA proteins to mediate NER activity was tested by incubating a plasmid containing a 1,3-cisplatin interstrand crosslink with a cell-free extract generated from XPA-deficient cells supplemented with purified wild-type or mutant XPA protein (Shivji, 1999). Addition of wild-type XPA protein to this mixture resulted in robust NER activity, as evidenced by formation of the characteristic excision products of 24-32 nts in length (FIG. 5A, lanes 1-2). By contrast no NER activity was observed following addition of the F75A or G73Δ/G74Δ mutants, while the G73Δ single deletion mutant displayed marginal NER activity. To test if these XPA mutations only affected binding to ERCC1, the DNA binding activities of wild-type and mutant XPA proteins were also compared. The binding of wild type and mutant XPA to a DNA three-way junction, representing a high affinity target for XPA in band-shift assays (Missura et al., 2001, EMBO J., 20, 3554-3564), was investigated. The wild-type, F75A, G73Δ and G73Δ/G74Δ XPA proteins all bound with similar affinity to a three-way junction (FIG. 5B), indicating that the mutant proteins are fully proficient in DNA binding and unlikely to be misfolded or otherwise inactive. These results show that single point mutations in XPA can result in a defect in NER activity by weakening the interaction between ERCC1 and XPA. Due to the highly cooperative nature of NER (Moggs et al., 1996), other NER functions and interactions may be disrupted as a result of blocking the recruitment of XPF-ERCC1.

Mutations in the XPA binding pocket of ERCC1 abolish NER in vitro. The specificity of inhibition of NER by XPA₆₇₋₈₀ was examined by observing changes in NER activity of ERCC1 resulting from mutations of single residues such as N110 or Y145. Mutant ERCC1 proteins were generated containing N110A or Y145A single mutations or a N110A/Y145A double mutation, and their activities compared to that of the wild-type ERCC1 protein. The ability of the ERCC1 proteins to mediate NER activity was tested by incubating a plasmid containing a 1,3-cisplatin interstrand crosslink with a cell-free extract generated from XPF-deficient cells (XPF cells are deficient in ERCC1 and XPF as the two proteins form an obligate heterodimer in human cells) as described above in assaying XPA mutants. While wild-type ERCC1, expressed as a heterodimer with XPF in baculovirus infected insect cells restored NER activity in mutant XPF cell extracts, ERCC1-Y145A and ERCC1-N110A only partially restored NER activity. An ERCC1 N110A/Y145A double mutant displayed only marginal NER activity. All XPF-ERCC1 proteins with mutations in the XPA binding domain displayed full nuclease activity on model substrates indicating that the proteins were properly folded. These studies demonstrate that these single and double point mutations in ERCC1 can result in a defect in NER activity in vitro by weakening the interaction between ERCC1 and XPA.

Mutations in the XPA binding pocket of ERCC1 abolish NER in living cells. The ERCC1-N110A/Y145A double mutant protein was tested for its ability to interact with XPA and support NER activity in living cells. The double mutant and wild-type ERCC1 proteins were introduced into ERCC1-deficient UV20 Chinese Hamster Ovary cells using a lentiviral expression system. Proper expression of the ERCC1 proteins was detected using immunofluorescent detection using an antibody against the HA tag present on the lentivirally expressed ERCC1 protein. Two main parameters were compared for the wild-type and mutant proteins. Global NER activity was assessed by measuring the ability of the wild-type and double mutant ERCC1 proteins to restore UV resistance in UV-20 cells. While expression of the wild-type protein fully restored UV resistance at doses of 1, 2, 4 and 10 (J/m²), the N110A/Y145A double mutant protein restored UV resistance to less than 50%, indicating that this ERCC1 form only partially restores NER activity in living cells. These studies demonstrate that the interaction between ERCC1 and XPA is required to mediate the NER reaction in living cells.

The interaction of ERCC1 and XPA was investigated in living cells as follows (Volker et al., 2001, Gillet and Schärer, 2006): Cells were irradiated with UV irradiation through polycarbonate filters that are only translucent to UV light through their 5 μM pores. This allows for the formation of spatially defined subnuclear region UV damage that can be detected as defined foci in fixed cells by antibodies against UV lesions in DNA as well as with antibodies against NER proteins recruited to these sites during the repair process. In UV-irradiated UV20 cells expressing wild-type ERCC1, XPA and ERCC1 colocalized at sites containing 6-4 photoproducts, indicating that both proteins are effectively recruited to UV-damaged sites in the nuclei where NER takes places. By contrast, in cells expressing ERCC1-N110A/Y145A only XPA, but not ERCC1 was found at sites of nuclear DNA damage, indicating that ERCC1 is not recruited to sites of active NER due to a lack of interaction with XPA. These studies show that mutations in the XPA binding pocket in ERCC1 abolish the interaction between XPA and ERCC1 in vivo, further validating this site as target for small molecules to inhibit NER.

Identification of small molecule inhibitors of the XPA-ERCC1 interaction. Several small molecule inhibitors of the XPA-ERCC1 interaction have been identified via high throughput screening using an in vitro binding assay. The XPA 14mer peptide was labeled with 6-carboxyfluorescein and bound to ERCC1. This peptide-protein complex was incubated with candidate small molecules, and inhibitors that block peptide binding were identified by a decrease in fluorescence polarization caused by the rapid rotational diffusion of the dissociated peptide. Approximately 20,000 commercially available compounds were screened, and 4 inhibitors have been identified that show a dose-dependent, saturable inhibition of XPA binding activity. Compound #1 (MW=329.37) exhibits an effective concentration for 50% inhibition (EC₅₀) of 20 μM. Compound #2 (MW=274.226) has an EC₅₀=60 μM. Compounds #3 (MW=336.42) and #4 (MW=305.38) both exhibit an EC₅₀=70 μM. The compounds were docked in silico and predicted to bind in the pocket that accepts the side chain of Y145. Chemical derivatives of these compounds are being designed and tested with the goal of improving potency.

XPA competes with single-stranded DNA for binding to ERCC1. Because XPA binds in the groove on ERCC1 (FIG. 2) that was previously implicated in DNA binding activity (Tsodikov et al., 2005), it was determined whether or not XPA competes with single-stranded DNA for binding to ERCC1. DNA binding activity was measured by monitoring fluorescence anisotropy, using single-stranded 40mer oligonucleotide labeled on the 5′ end with 6-carboxyfluorescein. The XPA₆₇₋₈₀ peptide does not detectably bind to DNA (not shown), although it does compete with DNA for binding to ERCC1 (FIG. 7). This result confirms that the DNA binding site on ERCC1's central domain overlaps with the XPA binding site. The EC₅₀ for binding of XPA₆₇₋₈₀ is in the micromolar range, but quenching of the fluorescent probe by high concentrations of XPA precluded an accurate measurement of the binding constant. An equilibrium binding constant of 1.5 μM for DNA binding to the central domain of ERCC1 has been reported (Tsodikov et al., 2005). By fitting the XPA competition titration data to a competitive binding model (see Eq. 9), the binding constant of for the XPA-ERCC1 complex was estimated to be K_{d}=(540±280) nM. Thus, XPA binds to the central domain of ERCC1 with approximately 3-fold higher affinity than single stranded DNA.

Inhibitors of XPA-ERCC1 complex formation. Molecular coordinate databases are screened for potential ligands of the XPA binding site of ERCC1 using Surflex (BioPharmics LLC) which implements the Hammerhead scoring function (Welch et al., 1996, Chem. Biol. 3:449). One database is the ICCB known bioactives collection (467 compounds). Another is the NCI diversity set (1,866 compounds) containing small drug-like compounds. A third is the Maybridge HitFinder collection (14,379 compounds). The compounds in the collection are drug-like, and have been selected to satisfy Lipinski's rule of five (not more than 5 hydrogen bond donors (OH and NH groups); not more than 10 hydrogen bond acceptors (notably N and O); molecular weight under 500 g/mol; partition coefficient log P less than 5). The compounds are also selected from a considerably larger set to eliminate clustering. Once a hit is found, there are, on average, ten closely related compounds in the larger set that can be screened for follow-up.

For virtual screening, coordinates of a 1.9 Å resolution structure of ERCC1 central domain (2A1I) are used together with the average structure of the XPA peptide as determined by X-ray crystallography in conjunction with NMR, as provided herein. Hits are identified and rank ordered using Surflex docking software. The structures and binding positions of the identified compounds emphasize the importance of ERCC1 amino acids, including Tyr145 and Tyr152, that interact with Gly-Gly-Gly motif in the XPA peptide.

### INCORPORATION BY REFERENCE

All of the patents and publications cited herein are hereby incorporated by reference in their entireties.

Those skilled in the art will recognize, or be able to ascertain using no more than routine experimentation, many equivalents to the specific embodiments of the invention described herein. Such equivalents are intended to be encompassed by the following claims.

TABLE 2

| | | | | | | | | | | | | |
|---|---|---|---|---|---|---|---|---|---|---|---|---|
| ATOM | 1 | CA | ASN | A | 99 | 13.984 | 5.746 | −3.509 | 1.00 | 40.00 | ercc | C |
| ATOM | 3 | CB | ASN | A | 99 | 14.229 | 5.347 | −2.056 | 1.00 | 40.00 | ercc | C |
| ATOM | 6 | CG | ASN | A | 99 | 15.458 | 4.470 | −1.884 | 1.00 | 40.00 | ercc | C |
| ATOM | 7 | OD1 | ASN | A | 99 | 15.780 | 3.634 | −2.734 | 1.00 | 40.00 | ercc | O |
| ATOM | 8 | ND2 | ASN | A | 99 | 16.150 | 4.652 | −0.769 | 1.00 | 40.00 | ercc | N |
| ATOM | 11 | C | ASN | A | 99 | 13.869 | 4.503 | −4.388 | 1.00 | 40.00 | ercc | C |
| ATOM | 12 | O | ASN | A | 99 | 14.347 | 4.476 | −5.510 | 1.00 | 40.00 | ercc | O |
| ATOM | 13 | N | ASN | A | 99 | 12.739 | 6.576 | −3.610 | 1.00 | 40.00 | ercc | N |
| ATOM | 17 | N | SER | A | 100 | 13.308 | 3.446 | −3.817 | 1.00 | 40.00 | ercc | N |
| ATOM | 19 | CA | SER | A | 100 | 12.393 | 2.636 | −4.573 | 1.00 | 40.00 | ercc | C |
| ATOM | 21 | CB | SER | A | 100 | 12.365 | 1.204 | −4.065 | 1.00 | 40.00 | ercc | C |
| ATOM | 24 | OG | SER | A | 100 | 11.856 | 1.125 | −2.744 | 1.00 | 40.00 | ercc | O |
| ATOM | 26 | C | SER | A | 100 | 11.104 | 3.396 | −4.291 | 1.00 | 40.00 | ercc | C |
| ATOM | 27 | O | SER | A | 100 | 10.812 | 3.733 | −3.135 | 1.00 | 40.00 | ercc | O |
| ATOM | 28 | N | ILE | A | 101 | 10.338 | 3.623 | −5.358 | 1.00 | 40.00 | ercc | N |
| ATOM | 30 | CA | ILE | A | 101 | 9.577 | 4.878 | −5.568 | 1.00 | 40.00 | ercc | C |
| ATOM | 32 | CB | ILE | A | 101 | 8.624 | 4.711 | −6.809 | 1.00 | 40.00 | ercc | C |
| ATOM | 34 | CG1 | ILE | A | 101 | 9.357 | 4.045 | −7.987 | 1.00 | 40.00 | ercc | C |
| ATOM | 37 | CG2 | ILE | A | 101 | 8.002 | 6.032 | −7.255 | 1.00 | 40.00 | ercc | C |
| ATOM | 41 | CD1 | ILE | A | 101 | 10.350 | 4.982 | −8.782 | 1.00 | 40.00 | ercc | C |
| ATOM | 45 | C | ILE | A | 101 | 8.796 | 5.478 | −4.382 | 1.00 | 40.00 | ercc | C |
| ATOM | 46 | O | ILE | A | 101 | 8.694 | 4.884 | −3.294 | 1.00 | 40.00 | ercc | O |
| ATOM | 47 | N | ILE | A | 102 | 8.268 | 6.680 | −4.613 | 1.00 | 40.00 | ercc | N |
| ATOM | 49 | CA | ILE | A | 102 | 7.146 | 7.187 | −3.836 | 1.00 | 40.00 | ercc | C |
| ATOM | 51 | CB | ILE | A | 102 | 7.540 | 8.337 | −2.875 | 1.00 | 40.00 | ercc | C |
| ATOM | 53 | CG1 | ILE | A | 102 | 8.843 | 8.035 | −2.116 | 1.00 | 40.00 | ercc | C |
| ATOM | 56 | CG2 | ILE | A | 102 | 6.391 | 8.671 | −1.929 | 1.00 | 40.00 | ercc | C |
| ATOM | 60 | CD1 | ILE | A | 102 | 8.722 | 7.034 | −0.961 | 1.00 | 40.00 | ercc | C |
| ATOM | 64 | C | ILE | A | 102 | 6.057 | 7.657 | −4.802 | 1.00 | 40.00 | ercc | C |
| ATOM | 65 | O | ILE | A | 102 | 6.320 | 8.431 | −5.722 | 1.00 | 40.00 | ercc | O |
| ATOM | 66 | N | VAL | A | 103 | 4.840 | 7.165 | −4.585 | 1.00 | 40.00 | ercc | N |
| ATOM | 68 | CA | VAL | A | 103 | 3.672 | 7.564 | −5.364 | 1.00 | 40.00 | ercc | C |
| ATOM | 70 | CB | VAL | A | 103 | 2.998 | 6.340 | −6.034 | 1.00 | 40.00 | ercc | C |
| ATOM | 72 | CG1 | VAL | A | 103 | 1.830 | 6.766 | −6.912 | 1.00 | 40.00 | ercc | C |
| ATOM | 76 | CG2 | VAL | A | 103 | 4.011 | 5.533 | −6.843 | 1.00 | 40.00 | ercc | C |
| ATOM | 80 | C | VAL | A | 103 | 2.671 | 8.237 | −4.430 | 1.00 | 40.00 | ercc | C |
| ATOM | 81 | O | VAL | A | 103 | 2.590 | 7.892 | −3.252 | 1.00 | 40.00 | ercc | O |
| ATOM | 82 | N | SER | A | 104 | 1.784 | 9.042 | −4.930 | 1.00 | 40.00 | ercc | N |
| ATOM | 84 | CA | SER | A | 104 | 0.707 | 9.572 | −4.021 | 1.00 | 40.00 | ercc | C |
| ATOM | 86 | CB | SER | A | 104 | 0.225 | 10.906 | −4.587 | 1.00 | 40.00 | ercc | C |
| ATOM | 89 | OG | SER | A | 104 | −1.174 | 11.054 | −4.291 | 1.00 | 40.00 | ercc | O |
| ATOM | 91 | C | SER | A | 104 | −0.483 | 8.612 | −4.020 | 1.00 | 40.00 | ercc | C |
| ATOM | 92 | O | SER | A | 104 | −0.824 | 8.063 | −5.050 | 1.00 | 40.00 | ercc | O |
| ATOM | 93 | N | PRO | A | 105 | −1.124 | 8.482 | −2.880 | 1.00 | 40.00 | ercc | N |
| ATOM | 94 | CA | PRO | A | 105 | −2.319 | 7.608 | −2.792 | 1.00 | 40.00 | ercc | C |
| ATOM | 96 | CB | PRO | A | 105 | −2.712 | 7.666 | −1.319 | 1.00 | 40.00 | ercc | C |
| ATOM | 99 | CG | PRO | A | 105 | −2.111 | 8.946 | −0.828 | 1.00 | 40.00 | ercc | C |
| ATOM | 102 | CD | PRO | A | 105 | −0.831 | 9.130 | −1.598 | 1.00 | 40.00 | ercc | C |
| ATOM | 105 | C | PRO | A | 105 | −3.385 | 8.166 | −3.712 | 1.00 | 40.00 | ercc | C |
| ATOM | 106 | O | PRO | A | 105 | −4.373 | 7.522 | −4.006 | 1.00 | 40.00 | ercc | O |
| ATOM | 107 | N | ARG | A | 106 | −3.147 | 9.351 | −4.209 | 1.00 | 40.00 | ercc | N |
| ATOM | 109 | CA | ARG | A | 106 | −4.083 | 9.943 | −5.162 | 1.00 | 40.00 | ercc | C |
| ATOM | 111 | CB | ARG | A | 106 | −3.796 | 11.438 | −5.160 | 1.00 | 40.00 | ercc | C |
| ATOM | 114 | CG | ARG | A | 106 | −4.841 | 12.135 | −6.023 | 1.00 | 40.00 | ercc | C |
| ATOM | 117 | CD | ARG | A | 106 | −5.078 | 13.549 | −5.498 | 1.00 | 40.00 | ercc | C |
| ATOM | 120 | NE | ARG | A | 106 | −4.323 | 14.439 | −6.425 | 1.00 | 40.00 | ercc | N |
| ATOM | 122 | CZ | ARG | A | 106 | −4.193 | 15.707 | −6.146 | 1.00 | 40.00 | ercc | C |
| ATOM | 123 | NH1 | ARG | A | 106 | −3.405 | 16.085 | −5.167 | 1.00 | 40.00 | ercc | N |
| ATOM | 126 | NH2 | ARG | A | 106 | −4.866 | 16.594 | −6.834 | 1.00 | 40.00 | ercc | N |
| ATOM | 129 | C | ARG | A | 106 | −3.783 | 9.337 | −6.519 | 1.00 | 40.00 | ercc | C |
| ATOM | 130 | O | ARG | A | 106 | −4.429 | 9.638 | −7.484 | 1.00 | 40.00 | ercc | O |
| ATOM | 131 | N | GLN | A | 107 | −2.810 | 8.467 | −6.596 | 1.00 | 40.00 | ercc | N |
| ATOM | 133 | CA | GLN | A | 107 | −2.490 | 7.824 | −7.891 | 1.00 | 40.00 | ercc | C |
| ATOM | 135 | CB | GLN | A | 107 | −0.963 | 7.777 | −7.955 | 1.00 | 40.00 | ercc | C |
| ATOM | 138 | CG | GLN | A | 107 | −0.458 | 8.645 | −9.105 | 1.00 | 40.00 | ercc | C |
| ATOM | 141 | CD | GLN | A | 107 | −1.112 | 8.203 | −10.403 | 1.00 | 40.00 | ercc | C |
| ATOM | 142 | OE1 | GLN | A | 107 | −1.785 | 7.202 | −10.448 | 1.00 | 40.00 | ercc | O |
| ATOM | 143 | NE2 | GLN | A | 107 | −0.942 | 8.920 | −11.464 | 1.00 | 40.00 | ercc | N |
| ATOM | 146 | C | GLN | A | 107 | −3.067 | 6.409 | −7.909 | 1.00 | 40.00 | ercc | C |
| ATOM | 147 | O | GLN | A | 107 | −3.003 | 5.722 | −8.904 | 1.00 | 40.00 | ercc | O |
| ATOM | 148 | N | ARG | A | 108 | −3.633 | 5.971 | −6.810 | 1.00 | 40.00 | ercc | N |
| ATOM | 150 | CA | ARG | A | 108 | −4.223 | 4.592 | −6.750 | 1.00 | 40.00 | ercc | C |
| ATOM | 152 | CB | ARG | A | 108 | −5.003 | 4.524 | −5.419 | 1.00 | 40.00 | ercc | C |
| ATOM | 155 | CG | ARG | A | 108 | −4.507 | 3.341 | −4.544 | 1.00 | 40.00 | ercc | C |
| ATOM | 158 | CD | ARG | A | 108 | −5.651 | 2.816 | −3.646 | 1.00 | 40.00 | ercc | C |
| ATOM | 161 | NE | ARG | A | 108 | −5.017 | 2.445 | −2.348 | 1.00 | 40.00 | ercc | N |
| ATOM | 163 | CZ | ARG | A | 108 | −4.462 | 3.356 | −1.604 | 1.00 | 40.00 | ercc | C |
| ATOM | 164 | NH1 | ARG | A | 108 | −4.911 | 4.581 | −1.618 | 1.00 | 40.00 | ercc | N |
| ATOM | 167 | NH2 | ARG | A | 108 | −3.453 | 3.045 | −0.847 | 1.00 | 40.00 | ercc | N |
| ATOM | 170 | C | ARG | A | 108 | −5.164 | 4.373 | −7.931 | 1.00 | 40.00 | ercc | C |
| ATOM | 171 | O | ARG | A | 108 | −5.265 | 5.196 | −8.814 | 1.00 | 40.00 | ercc | O |
| ATOM | 172 | N | GLY | A | 109 | −5.845 | 3.268 | −7.962 | 1.00 | 40.00 | ercc | N |
| ATOM | 174 | CA | GLY | A | 109 | −6.752 | 3.010 | −9.106 | 1.00 | 40.00 | ercc | C |
| ATOM | 177 | C | GLY | A | 109 | −5.908 | 3.043 | −10.375 | 1.00 | 40.00 | ercc | C |
| ATOM | 178 | O | GLY | A | 109 | −5.477 | 2.025 | −10.861 | 1.00 | 40.00 | ercc | O |
| ATOM | 179 | N | ASN | A | 110 | −5.659 | 4.213 | −10.896 | 1.00 | 40.00 | ercc | N |
| ATOM | 181 | CA | ASN | A | 110 | −4.805 | 4.357 | −12.135 | 1.00 | 40.00 | ercc | C |
| ATOM | 183 | CB | ASN | A | 110 | −3.779 | 5.397 | −11.740 | 1.00 | 40.00 | ercc | C |
| ATOM | 186 | CG | ASN | A | 110 | −2.962 | 5.747 | −12.966 | 1.00 | 40.00 | ercc | C |
| ATOM | 187 | OD1 | ASN | A | 110 | −2.564 | 4.874 | −13.708 | 1.00 | 40.00 | ercc | O |
| ATOM | 188 | ND2 | ASN | A | 110 | −2.698 | 6.991 | −13.220 | 1.00 | 40.00 | ercc | N |
| ATOM | 191 | C | ASN | A | 110 | −4.064 | 3.054 | −12.515 | 1.00 | 40.00 | ercc | C |
| ATOM | 192 | O | ASN | A | 110 | −3.205 | 2.625 | −11.775 | 1.00 | 40.00 | ercc | O |
| ATOM | 193 | N | PRO | A | 111 | −4.426 | 2.474 | −13.659 | 1.00 | 40.00 | ercc | N |
| ATOM | 194 | CA | PRO | A | 111 | −3.815 | 1.182 | −14.154 | 1.00 | 40.00 | ercc | C |
| ATOM | 196 | CB | PRO | A | 111 | −4.611 | 0.854 | −15.410 | 1.00 | 40.00 | ercc | C |
| ATOM | 199 | CG | PRO | A | 111 | −5.137 | 2.169 | −15.861 | 1.00 | 40.00 | ercc | C |
| ATOM | 202 | CD | PRO | A | 111 | −5.429 | 2.958 | −14.608 | 1.00 | 40.00 | ercc | C |
| ATOM | 205 | C | PRO | A | 111 | −2.337 | 1.308 | −14.497 | 1.00 | 40.00 | ercc | C |
| ATOM | 206 | O | PRO | A | 111 | −1.628 | 0.334 | −14.590 | 1.00 | 40.00 | ercc | O |
| ATOM | 207 | N | VAL | A | 112 | −1.860 | 2.477 | −14.663 | 1.00 | 40.00 | ercc | N |
| ATOM | 209 | CA | VAL | A | 112 | −0.438 | 2.641 | −14.952 | 1.00 | 40.00 | ercc | C |
| ATOM | 211 | CB | VAL | A | 112 | −0.050 | 4.142 | −15.054 | 1.00 | 40.00 | ercc | C |
| ATOM | 213 | CG1 | VAL | A | 112 | 1.462 | 4.321 | −15.145 | 1.00 | 40.00 | ercc | C |
| ATOM | 217 | CG2 | VAL | A | 112 | −0.721 | 4.783 | −16.261 | 1.00 | 40.00 | ercc | C |
| ATOM | 221 | C | VAL | A | 112 | 0.407 | 1.939 | −13.889 | 1.00 | 40.00 | ercc | C |
| ATOM | 222 | O | VAL | A | 112 | 1.424 | 1.319 | −14.208 | 1.00 | 40.00 | ercc | O |
| ATOM | 223 | N | LEU | A | 113 | −0.034 | 2.028 | −12.635 | 1.00 | 40.00 | ercc | N |
| ATOM | 225 | CA | LEU | A | 113 | 0.654 | 1.396 | −11.507 | 1.00 | 40.00 | ercc | C |
| ATOM | 227 | CB | LEU | A | 113 | −0.091 | 1.671 | −10.199 | 1.00 | 40.00 | ercc | C |
| ATOM | 230 | CG | LEU | A | 113 | −0.002 | 3.101 | −9.657 | 1.00 | 40.00 | ercc | C |
| ATOM | 232 | CD1 | LEU | A | 113 | −1.114 | 3.366 | −8.653 | 1.00 | 40.00 | ercc | C |
| ATOM | 236 | CD2 | LEU | A | 113 | 1.368 | 3.398 | −9.048 | 1.00 | 40.00 | ercc | C |
| ATOM | 240 | C | LEU | A | 113 | 0.836 | −0.106 | −11.700 | 1.00 | 40.00 | ercc | C |
| ATOM | 241 | O | LEU | A | 113 | 1.680 | −0.727 | −11.049 | 1.00 | 40.00 | ercc | O |
| ATOM | 242 | N | LYS | A | 114 | 0.040 | −0.675 | −12.602 | 1.00 | 40.00 | ercc | N |
| ATOM | 244 | CA | LYS | A | 114 | 0.128 | −2.092 | −12.939 | 1.00 | 40.00 | ercc | C |
| ATOM | 246 | CB | LYS | A | 114 | −1.145 | −2.573 | −13.648 | 1.00 | 40.00 | ercc | C |
| ATOM | 249 | CG | LYS | A | 114 | −2.453 | −2.264 | −12.920 | 1.00 | 40.00 | ercc | C |
| ATOM | 252 | CD | LYS | A | 114 | −3.673 | −2.875 | −13.619 | 1.00 | 40.00 | ercc | C |
| ATOM | 255 | CE | LYS | A | 114 | −3.816 | −2.413 | −15.068 | 1.00 | 40.00 | ercc | C |
| ATOM | 258 | NZ | LYS | A | 114 | −5.198 | −2.621 | −15.597 | 1.00 | 40.00 | ercc | N |
| ATOM | 262 | C | LYS | A | 114 | 1.344 | −2.393 | −13.810 | 1.00 | 40.00 | ercc | C |
| ATOM | 263 | O | LYS | A | 114 | 1.759 | −3.547 | −13.917 | 1.00 | 40.00 | ercc | O |
| ATOM | 264 | N | PHE | A | 115 | 1.910 | −1.364 | −14.431 | 1.00 | 40.00 | ercc | N |
| ATOM | 266 | CA | PHE | A | 115 | 3.046 | −1.556 | −15.329 | 1.00 | 40.00 | ercc | C |
| ATOM | 268 | CB | PHE | A | 115 | 2.738 | −0.955 | −16.697 | 1.00 | 40.00 | ercc | C |
| ATOM | 271 | CG | PHE | A | 115 | 1.474 | −1.492 | −17.303 | 1.00 | 40.00 | ercc | C |
| ATOM | 272 | CD1 | PHE | A | 115 | 0.267 | −0.814 | −17.143 | 1.00 | 40.00 | ercc | C |
| ATOM | 274 | CD2 | PHE | A | 115 | 1.478 | −2.695 | −18.002 | 1.00 | 40.00 | ercc | C |
| ATOM | 276 | CE1 | PHE | A | 115 | −0.910 | −1.313 | −17.693 | 1.00 | 40.00 | ercc | C |
| ATOM | 278 | CE2 | PHE | A | 115 | 0.306 | −3.204 | −18.555 | 1.00 | 40.00 | ercc | C |
| ATOM | 280 | CZ | PHE | A | 115 | −0.891 | −2.511 | −18.399 | 1.00 | 40.00 | ercc | C |
| ATOM | 282 | C | PHE | A | 115 | 4.331 | −1.020 | −14.715 | 1.00 | 40.00 | ercc | C |
| ATOM | 283 | O | PHE | A | 115 | 5.369 | −0.920 | −15.375 | 1.00 | 40.00 | ercc | O |
| ATOM | 284 | N | VAL | A | 116 | 4.234 | −0.688 | −13.433 | 1.00 | 40.00 | ercc | N |
| ATOM | 286 | CA | VAL | A | 116 | 5.388 | −0.401 | −12.601 | 1.00 | 40.00 | ercc | C |
| ATOM | 288 | CB | VAL | A | 116 | 5.078 | 0.693 | −11.553 | 1.00 | 40.00 | ercc | C |
| ATOM | 290 | CG1 | VAL | A | 116 | 6.368 | 1.277 | −11.005 | 1.00 | 40.00 | ercc | C |
| ATOM | 294 | CG2 | VAL | A | 116 | 4.235 | 1.800 | −12.167 | 1.00 | 40.00 | ercc | C |
| ATOM | 298 | C | VAL | A | 116 | 5.764 | −1.723 | −11.928 | 1.00 | 40.00 | ercc | C |
| ATOM | 299 | O | VAL | A | 116 | 5.425 | −1.971 | −10.767 | 1.00 | 40.00 | ercc | O |
| ATOM | 300 | N | ARG | A | 117 | 6.458 | −2.570 | −12.686 | 1.00 | 40.00 | ercc | N |
| ATOM | 302 | CA | ARG | A | 117 | 6.736 | −3.949 | −12.282 | 1.00 | 40.00 | ercc | C |
| ATOM | 304 | CB | ARG | A | 117 | 6.247 | −4.927 | −13.353 | 1.00 | 40.00 | ercc | C |
| ATOM | 307 | CG | ARG | A | 117 | 4.764 | −4.852 | −13.667 | 1.00 | 40.00 | ercc | C |
| ATOM | 310 | CD | ARG | A | 117 | 4.490 | −5.392 | −15.060 | 1.00 | 40.00 | ercc | C |
| ATOM | 313 | NE | ARG | A | 117 | 5.115 | −4.562 | −16.090 | 1.00 | 40.00 | ercc | N |
| ATOM | 315 | CZ | ARG | A | 117 | 5.445 | −4.985 | −17.308 | 1.00 | 40.00 | ercc | C |
| ATOM | 316 | NH1 | ARG | A | 117 | 5.222 | −6.242 | −17.672 | 1.00 | 40.00 | ercc | N |
| ATOM | 319 | NH2 | ARG | A | 117 | 6.010 | −4.145 | −18.168 | 1.00 | 40.00 | ercc | N |
| ATOM | 322 | C | ARG | A | 117 | 8.216 | −4.207 | −12.028 | 1.00 | 40.00 | ercc | C |
| ATOM | 323 | O | ARG | A | 117 | 8.573 | −4.932 | −11.096 | 1.00 | 40.00 | ercc | O |
| ATOM | 324 | N | ASN | A | 118 | 9.067 | −3.623 | −12.866 | 1.00 | 40.00 | ercc | N |
| ATOM | 326 | CA | ASN | A | 118 | 10.508 | −3.853 | −12.796 | 1.00 | 40.00 | ercc | C |
| ATOM | 328 | CB | ASN | A | 118 | 11.183 | −3.406 | −14.099 | 1.00 | 40.00 | ercc | C |
| ATOM | 331 | CG | ASN | A | 118 | 10.609 | −4.098 | −15.331 | 1.00 | 40.00 | ercc | C |
| ATOM | 332 | OD1 | ASN | A | 118 | 10.422 | −5.315 | −15.348 | 1.00 | 40.00 | ercc | O |
| ATOM | 333 | ND2 | ASN | A | 118 | 10.339 | −3.319 | −16.373 | 1.00 | 40.00 | ercc | N |
| ATOM | 336 | C | ASN | A | 118 | 11.161 | −3.179 | −11.589 | 1.00 | 40.00 | ercc | C |
| ATOM | 337 | O | ASN | A | 118 | 12.279 | −3.530 | −11.201 | 1.00 | 40.00 | ercc | O |
| ATOM | 338 | N | VAL | A | 119 | 10.451 | −2.218 | −10.997 | 1.00 | 40.00 | ercc | N |
| ATOM | 340 | CA | VAL | A | 119 | 10.968 | −1.435 | −9.872 | 1.00 | 40.00 | ercc | C |
| ATOM | 342 | CB | VAL | A | 119 | 11.312 | 0.033 | −10.284 | 1.00 | 40.00 | ercc | C |
| ATOM | 344 | CG1 | VAL | A | 119 | 12.444 | 0.067 | −11.311 | 1.00 | 40.00 | ercc | C |
| ATOM | 348 | CG2 | VAL | A | 119 | 10.081 | 0.778 | −10.805 | 1.00 | 40.00 | ercc | C |
| ATOM | 352 | C | VAL | A | 119 | 10.012 | −1.417 | −8.673 | 1.00 | 40.00 | ercc | C |
| ATOM | 353 | O | VAL | A | 119 | 8.791 | −1.370 | −8.852 | 1.00 | 40.00 | ercc | O |
| ATOM | 354 | N | PRO | A | 120 | 10.566 | −1.483 | −7.448 | 1.00 | 40.00 | ercc | N |
| ATOM | 355 | CA | PRO | A | 120 | 9.766 | −1.274 | −6.240 | 1.00 | 40.00 | ercc | C |
| ATOM | 357 | CB | PRO | A | 120 | 10.764 | −1.524 | −5.106 | 1.00 | 40.00 | ercc | C |
| ATOM | 360 | CG | PRO | A | 120 | 12.112 | −1.366 | −5.728 | 1.00 | 40.00 | ercc | C |
| ATOM | 363 | CD | PRO | A | 120 | 11.971 | −1.805 | −7.135 | 1.00 | 40.00 | ercc | C |
| ATOM | 366 | C | PRO | A | 120 | 9.190 | 0.142 | −6.156 | 1.00 | 40.00 | ercc | C |
| ATOM | 367 | O | PRO | A | 120 | 9.866 | 1.115 | −6.501 | 1.00 | 40.00 | ercc | O |
| ATOM | 368 | N | TRP | A | 121 | 7.940 | 0.233 | −5.712 | 1.00 | 40.00 | ercc | N |
| ATOM | 370 | CA | TRP | A | 121 | 7.262 | 1.511 | −5.533 | 1.00 | 40.00 | ercc | C |
| ATOM | 372 | CB | TRP | A | 121 | 6.512 | 1.912 | −6.810 | 1.00 | 40.00 | ercc | C |
| ATOM | 375 | CG | TRP | A | 121 | 5.336 | 1.044 | −7.169 | 1.00 | 40.00 | ercc | C |
| ATOM | 376 | CD1 | TRP | A | 121 | 5.340 | −0.040 | −8.000 | 1.00 | 40.00 | ercc | C |
| ATOM | 378 | CD2 | TRP | A | 121 | 3.984 | 1.202 | −6.724 | 1.00 | 40.00 | ercc | C |
| ATOM | 379 | NE1 | TRP | A | 121 | 4.075 | −0.570 | −8.098 | 1.00 | 40.00 | ercc | N |
| ATOM | 381 | CE2 | TRP | A | 121 | 3.223 | 0.173 | −7.322 | 1.00 | 40.00 | ercc | C |
| ATOM | 382 | CE3 | TRP | A | 121 | 3.340 | 2.110 | −5.873 | 1.00 | 40.00 | ercc | C |
| ATOM | 384 | CZ2 | TRP | A | 121 | 1.850 | 0.030 | −7.102 | 1.00 | 40.00 | ercc | C |
| ATOM | 386 | CZ3 | TRP | A | 121 | 1.978 | 1.964 | −5.648 | 1.00 | 40.00 | ercc | C |
| ATOM | 388 | CH2 | TRP | A | 121 | 1.248 | 0.929 | −6.260 | 1.00 | 40.00 | ercc | C |
| ATOM | 390 | C | TRP | A | 121 | 6.309 | 1.442 | −4.347 | 1.00 | 40.00 | ercc | C |
| ATOM | 391 | O | TRP | A | 121 | 5.838 | 0.364 | −3.989 | 1.00 | 40.00 | ercc | O |
| ATOM | 392 | N | GLU | A | 122 | 6.027 | 2.590 | −3.740 | 1.00 | 40.00 | ercc | N |
| ATOM | 394 | CA | GLU | A | 122 | 5.089 | 2.648 | −2.622 | 1.00 | 40.00 | ercc | C |
| ATOM | 396 | CB | GLU | A | 122 | 5.794 | 2.353 | −1.288 | 1.00 | 40.00 | ercc | C |
| ATOM | 399 | CG | GLU | A | 122 | 6.868 | 3.362 | −0.880 | 1.00 | 40.00 | ercc | C |
| ATOM | 402 | CD | GLU | A | 122 | 7.425 | 3.100 | 0.511 | 1.00 | 40.00 | ercc | C |
| ATOM | 403 | OE1 | GLU | A | 122 | 7.407 | 4.033 | 1.343 | 1.00 | 40.00 | ercc | O |
| ATOM | 404 | OE2 | GLU | A | 122 | 7.877 | 1.966 | 0.775 | 1.00 | 40.00 | ercc | O |
| ATOM | 405 | C | GLU | A | 122 | 4.355 | 3.981 | −2.556 | 1.00 | 40.00 | ercc | C |
| ATOM | 406 | O | GLU | A | 122 | 4.829 | 4.988 | −3.080 | 1.00 | 40.00 | ercc | O |
| ATOM | 407 | N | PHE | A | 123 | 3.192 | 3.971 | −1.910 | 1.00 | 40.00 | ercc | N |
| ATOM | 409 | CA | PHE | A | 123 | 2.454 | 5.196 | −1.641 | 1.00 | 40.00 | ercc | C |
| ATOM | 411 | CB | PHE | A | 123 | 0.987 | 4.907 | −1.309 | 1.00 | 40.00 | ercc | C |
| ATOM | 414 | CG | PHE | A | 123 | 0.186 | 4.375 | −2.466 | 1.00 | 40.00 | ercc | C |
| ATOM | 415 | CD1 | PHE | A | 123 | 0.330 | 4.909 | −3.746 | 1.00 | 40.00 | ercc | C |
| ATOM | 417 | CD2 | PHE | A | 123 | −0.738 | 3.357 | −2.265 | 1.00 | 40.00 | ercc | C |
| ATOM | 419 | CE1 | PHE | A | 123 | −0.421 | 4.418 | −4.810 | 1.00 | 40.00 | ercc | C |
| ATOM | 421 | CE2 | PHE | A | 123 | −1.494 | 2.862 | −3.323 | 1.00 | 40.00 | ercc | C |
| ATOM | 423 | CZ | PHE | A | 123 | −1.336 | 3.392 | −4.597 | 1.00 | 40.00 | ercc | C |
| ATOM | 425 | C | PHE | A | 123 | 3.089 | 5.951 | −0.485 | 1.00 | 40.00 | ercc | C |
| ATOM | 426 | O | PHE | A | 123 | 3.340 | 5.382 | 0.580 | 1.00 | 40.00 | ercc | O |
| ATOM | 427 | N | GLY | A | 124 | 3.351 | 7.231 | −0.704 | 1.00 | 40.00 | ercc | N |
| ATOM | 429 | CA | GLY | A | 124 | 3.829 | 8.103 | 0.352 | 1.00 | 40.00 | ercc | C |
| ATOM | 432 | C | GLY | A | 124 | 3.065 | 9.404 | 0.319 | 1.00 | 40.00 | ercc | C |
| ATOM | 433 | O | GLY | A | 124 | 2.349 | 9.692 | −0.643 | 1.00 | 40.00 | ercc | O |
| ATOM | 434 | N | ASP | A | 125 | 3.214 | 10.192 | 1.375 | 1.00 | 40.00 | ercc | N |
| ATOM | 436 | CA | ASP | A | 125 | 2.602 | 11.507 | 1.437 | 1.00 | 40.00 | ercc | C |
| ATOM | 438 | CB | ASP | A | 125 | 2.263 | 11.858 | 2.891 | 1.00 | 40.00 | ercc | C |
| ATOM | 441 | CG | ASP | A | 125 | 0.957 | 12.627 | 3.029 | 1.00 | 40.00 | ercc | C |
| ATOM | 442 | OD1 | ASP | A | 125 | 0.344 | 12.992 | 2.002 | 1.00 | 40.00 | ercc | O |
| ATOM | 443 | OD2 | ASP | A | 125 | 0.544 | 12.863 | 4.182 | 1.00 | 40.00 | ercc | O |
| ATOM | 444 | C | ASP | A | 125 | 3.572 | 12.524 | 0.829 | 1.00 | 40.00 | ercc | C |
| ATOM | 445 | O | ASP | A | 125 | 4.392 | 13.113 | 1.541 | 1.00 | 40.00 | ercc | O |
| ATOM | 446 | N | VAL | A | 126 | 3.492 | 12.709 | −0.489 | 1.00 | 40.00 | ercc | N |
| ATOM | 448 | CA | VAL | A | 126 | 4.373 | 13.651 | −1.201 | 1.00 | 40.00 | ercc | C |
| ATOM | 450 | CB | VAL | A | 126 | 5.499 | 12.935 | −2.005 | 1.00 | 40.00 | ercc | C |
| ATOM | 452 | CG1 | VAL | A | 126 | 6.560 | 12.363 | −1.070 | 1.00 | 40.00 | ercc | C |
| ATOM | 456 | CG2 | VAL | A | 126 | 4.928 | 11.862 | −2.932 | 1.00 | 40.00 | ercc | C |
| ATOM | 460 | C | VAL | A | 126 | 3.616 | 14.611 | −2.125 | 1.00 | 40.00 | ercc | C |
| ATOM | 461 | O | VAL | A | 126 | 2.549 | 14.273 | −2.647 | 1.00 | 40.00 | ercc | O |
| ATOM | 462 | N | ILE | A | 127 | 4.185 | 15.801 | −2.319 | 1.00 | 40.00 | ercc | N |
| ATOM | 464 | CA | ILE | A | 127 | 3.586 | 16.840 | −3.166 | 1.00 | 40.00 | ercc | C |
| ATOM | 466 | CB | ILE | A | 127 | 4.390 | 18.175 | −3.134 | 1.00 | 40.00 | ercc | C |
| ATOM | 468 | CG1 | ILE | A | 127 | 4.697 | 18.591 | −1.690 | 1.00 | 40.00 | ercc | C |
| ATOM | 471 | CG2 | ILE | A | 127 | 3.619 | 19.280 | −3.855 | 1.00 | 40.00 | ercc | C |
| ATOM | 475 | CD1 | ILE | A | 127 | 5.765 | 19.667 | −1.554 | 1.00 | 40.00 | ercc | C |
| ATOM | 479 | C | ILE | A | 127 | 3.367 | 16.387 | −4.620 | 1.00 | 40.00 | ercc | C |
| ATOM | 480 | O | ILE | A | 127 | 2.260 | 16.540 | −5.143 | 1.00 | 40.00 | ercc | O |
| ATOM | 481 | N | PRO | A | 128 | 4.405 | 15.819 | −5.274 | 1.00 | 40.00 | ercc | N |
| ATOM | 482 | CA | PRO | A | 128 | 4.188 | 15.453 | −6.671 | 1.00 | 40.00 | ercc | C |
| ATOM | 484 | CB | PRO | A | 128 | 5.611 | 15.246 | −7.191 | 1.00 | 40.00 | ercc | C |
| ATOM | 487 | CG | PRO | A | 128 | 6.358 | 14.760 | −6.008 | 1.00 | 40.00 | ercc | C |
| ATOM | 490 | CD | PRO | A | 128 | 5.772 | 15.480 | −4.828 | 1.00 | 40.00 | ercc | C |
| ATOM | 493 | C | PRO | A | 128 | 3.384 | 14.164 | −6.807 | 1.00 | 40.00 | ercc | C |
| ATOM | 494 | O | PRO | A | 128 | 2.870 | 13.640 | −5.814 | 1.00 | 40.00 | ercc | O |
| ATOM | 495 | N | ASP | A | 129 | 3.273 | 13.668 | −8.034 | 1.00 | 40.00 | ercc | N |
| ATOM | 497 | CA | ASP | A | 129 | 2.686 | 12.362 | −8.271 | 1.00 | 40.00 | ercc | C |
| ATOM | 499 | CB | ASP | A | 129 | 2.227 | 12.226 | −9.726 | 1.00 | 40.00 | ercc | C |
| ATOM | 502 | CG | ASP | A | 129 | 0.885 | 12.889 | −9.982 | 1.00 | 40.00 | ercc | C |
| ATOM | 503 | OD1 | ASP | A | 129 | 0.056 | 12.946 | −9.051 | 1.00 | 40.00 | ercc | O |
| ATOM | 504 | OD2 | ASP | A | 129 | 0.647 | 13.350 | −11.116 | 1.00 | 40.00 | ercc | O |
| ATOM | 505 | C | ASP | A | 129 | 3.700 | 11.285 | −7.903 | 1.00 | 40.00 | ercc | C |
| ATOM | 506 | O | ASP | A | 129 | 3.415 | 10.420 | −7.071 | 1.00 | 40.00 | ercc | O |
| ATOM | 507 | N | TYR | A | 130 | 4.886 | 11.361 | −8.503 | 1.00 | 40.00 | ercc | N |
| ATOM | 509 | CA | TYR | A | 130 | 5.924 | 10.360 | −8.279 | 1.00 | 40.00 | ercc | C |
| ATOM | 511 | CB | TYR | A | 130 | 6.094 | 9.474 | −9.515 | 1.00 | 40.00 | ercc | C |
| ATOM | 514 | CG | TYR | A | 130 | 4.786 | 8.942 | −10.022 | 1.00 | 40.00 | ercc | C |
| ATOM | 515 | CD1 | TYR | A | 130 | 4.244 | 7.772 | −9.505 | 1.00 | 40.00 | ercc | C |
| ATOM | 517 | CD2 | TYR | A | 130 | 4.069 | 9.628 | −10.999 | 1.00 | 40.00 | ercc | C |
| ATOM | 519 | CE1 | TYR | A | 130 | 3.029 | 7.287 | −9.960 | 1.00 | 40.00 | ercc | C |
| ATOM | 521 | CE2 | TYR | A | 130 | 2.849 | 9.155 | −11.456 | 1.00 | 40.00 | ercc | C |
| ATOM | 523 | CZ | TYR | A | 130 | 2.335 | 7.984 | −10.930 | 1.00 | 40.00 | ercc | C |
| ATOM | 524 | OH | TYR | A | 130 | 1.133 | 7.499 | −11.386 | 1.00 | 40.00 | ercc | O |
| ATOM | 526 | C | TYR | A | 130 | 7.253 | 10.983 | −7.902 | 1.00 | 40.00 | ercc | C |
| ATOM | 527 | O | TYR | A | 130 | 7.766 | 11.852 | −8.606 | 1.00 | 40.00 | ercc | O |
| ATOM | 528 | N | VAL | A | 131 | 7.794 | 10.535 | −6.773 | 1.00 | 40.00 | ercc | N |
| ATOM | 530 | CA | VAL | A | 131 | 9.152 | 10.877 | −6.376 | 1.00 | 40.00 | ercc | C |
| ATOM | 532 | CB | VAL | A | 131 | 9.302 | 10.984 | −4.837 | 1.00 | 40.00 | ercc | C |
| ATOM | 534 | CG1 | VAL | A | 131 | 10.770 | 11.074 | −4.428 | 1.00 | 40.00 | ercc | C |
| ATOM | 538 | CG2 | VAL | A | 131 | 8.530 | 12.184 | −4.307 | 1.00 | 40.00 | ercc | C |
| ATOM | 542 | C | VAL | A | 131 | 10.083 | 9.812 | −6.942 | 1.00 | 40.00 | ercc | C |
| ATOM | 543 | O | VAL | A | 131 | 9.941 | 8.623 | −6.646 | 1.00 | 40.00 | ercc | O |
| ATOM | 544 | N | LEU | A | 132 | 11.029 | 10.252 | −7.768 | 1.00 | 40.00 | ercc | N |
| ATOM | 546 | CA | LEU | A | 132 | 11.945 | 9.347 | −8.460 | 1.00 | 40.00 | ercc | C |
| ATOM | 548 | CB | LEU | A | 132 | 11.888 | 9.599 | −9.974 | 1.00 | 40.00 | ercc | C |
| ATOM | 551 | CG | LEU | A | 132 | 10.883 | 8.828 | −10.850 | 1.00 | 40.00 | ercc | C |
| ATOM | 553 | CD1 | LEU | A | 132 | 9.498 | 8.684 | −10.218 | 1.00 | 40.00 | ercc | C |
| ATOM | 557 | CD2 | LEU | A | 132 | 10.760 | 9.520 | −12.201 | 1.00 | 40.00 | ercc | C |
| ATOM | 561 | C | LEU | A | 132 | 13.384 | 9.449 | −7.942 | 1.00 | 40.00 | ercc | C |
| ATOM | 562 | O | LEU | A | 132 | 14.213 | 8.579 | −8.219 | 1.00 | 40.00 | ercc | O |
| ATOM | 563 | N | GLY | A | 133 | 13.661 | 10.510 | −7.185 | 1.00 | 40.00 | ercc | N |
| ATOM | 565 | CA | GLY | A | 133 | 14.980 | 10.757 | −6.589 | 1.00 | 40.00 | ercc | C |
| ATOM | 568 | C | GLY | A | 133 | 14.908 | 11.942 | −5.640 | 1.00 | 40.00 | ercc | C |
| ATOM | 569 | O | GLY | A | 133 | 13.866 | 12.601 | −5.560 | 1.00 | 40.00 | ercc | O |
| ATOM | 570 | N | GLN | A | 134 | 16.002 | 12.225 | −4.926 | 1.00 | 40.00 | ercc | N |
| ATOM | 572 | CA | GLN | A | 134 | 16.026 | 13.310 | −3.923 | 1.00 | 40.00 | ercc | C |
| ATOM | 574 | CB | GLN | A | 134 | 17.446 | 13.552 | −3.368 | 1.00 | 40.00 | ercc | C |
| ATOM | 577 | CG | GLN | A | 134 | 17.620 | 14.912 | −2.650 | 1.00 | 40.00 | ercc | C |
| ATOM | 580 | CD | GLN | A | 134 | 18.635 | 14.903 | −1.509 | 1.00 | 40.00 | ercc | C |
| ATOM | 581 | OE1 | GLN | A | 134 | 19.135 | 13.853 | −1.103 | 1.00 | 40.00 | ercc | O |
| ATOM | 582 | NE2 | GLN | A | 134 | 18.933 | 16.088 | −0.982 | 1.00 | 40.00 | ercc | N |
| ATOM | 585 | C | GLN | A | 134 | 15.410 | 14.615 | −4.431 | 1.00 | 40.00 | ercc | C |
| ATOM | 586 | O | GLN | A | 134 | 14.739 | 15.328 | −3.678 | 1.00 | 40.00 | ercc | O |
| ATOM | 587 | N | SER | A | 135 | 15.631 | 14.914 | −5.709 | 1.00 | 40.00 | ercc | N |
| ATOM | 589 | CA | SER | A | 135 | 15.116 | 16.139 | −6.297 | 1.00 | 40.00 | ercc | C |
| ATOM | 591 | CB | SER | A | 135 | 16.210 | 17.211 | −6.290 | 1.00 | 40.00 | ercc | C |
| ATOM | 594 | OG | SER | A | 135 | 15.703 | 18.456 | −5.834 | 1.00 | 40.00 | ercc | O |
| ATOM | 596 | C | SER | A | 135 | 14.547 | 15.907 | −7.705 | 1.00 | 40.00 | ercc | C |
| ATOM | 597 | O | SER | A | 135 | 14.405 | 16.839 | −8.488 | 1.00 | 40.00 | ercc | O |
| ATOM | 598 | N | THR | A | 136 | 14.223 | 14.653 | −8.017 | 1.00 | 40.00 | ercc | N |
| ATOM | 600 | CA | THR | A | 136 | 13.512 | 14.313 | −9.248 | 1.00 | 40.00 | ercc | C |
| ATOM | 602 | CB | THR | A | 136 | 14.164 | 13.123 | −9.987 | 1.00 | 40.00 | ercc | C |
| ATOM | 604 | OG1 | THR | A | 136 | 15.554 | 13.397 | −10.211 | 1.00 | 40.00 | ercc | O |
| ATOM | 606 | CG2 | THR | A | 136 | 13.478 | 12.851 | −11.328 | 1.00 | 40.00 | ercc | C |
| ATOM | 610 | C | THR | A | 136 | 12.074 | 13.953 | −8.901 | 1.00 | 40.00 | ercc | C |
| ATOM | 611 | O | THR | A | 136 | 11.830 | 13.200 | −7.955 | 1.00 | 40.00 | ercc | O |
| ATOM | 612 | N | CYS | A | 137 | 11.127 | 14.498 | −9.660 | 1.00 | 40.00 | ercc | N |
| ATOM | 614 | CA | CYS | A | 137 | 9.718 | 14.181 | −9.463 | 1.00 | 40.00 | ercc | C |
| ATOM | 616 | CB | CYS | A | 137 | 9.100 | 15.085 | −8.394 | 1.00 | 40.00 | ercc | C |
| ATOM | 619 | SG | CYS | A | 137 | 8.785 | 16.780 | −8.924 | 1.00 | 40.00 | ercc | S |
| ATOM | 621 | C | CYS | A | 137 | 8.917 | 14.261 | −10.757 | 1.00 | 40.00 | ercc | C |
| ATOM | 622 | O | CYS | A | 137 | 9.305 | 14.951 | −11.703 | 1.00 | 40.00 | ercc | O |
| ATOM | 623 | N | ALA | A | 138 | 7.794 | 13.548 | −10.780 | 1.00 | 40.00 | ercc | N |
| ATOM | 625 | CA | ALA | A | 138 | 6.943 | 13.477 | −11.957 | 1.00 | 40.00 | ercc | C |
| ATOM | 627 | CB | ALA | A | 138 | 7.074 | 12.115 | −12.624 | 1.00 | 40.00 | ercc | C |
| ATOM | 631 | C | ALA | A | 138 | 5.486 | 13.778 | −11.631 | 1.00 | 40.00 | ercc | C |
| ATOM | 632 | O | ALA | A | 138 | 5.055 | 13.663 | −10.480 | 1.00 | 40.00 | ercc | O |
| ATOM | 633 | N | LEU | A | 139 | 4.743 | 14.170 | −12.660 | 1.00 | 40.00 | ercc | N |
| ATOM | 635 | CA | LEU | A | 139 | 3.320 | 14.432 | −12.541 | 1.00 | 40.00 | ercc | C |
| ATOM | 637 | CB | LEU | A | 139 | 3.060 | 15.939 | −12.555 | 1.00 | 40.00 | ercc | C |
| ATOM | 640 | CG | LEU | A | 139 | 2.081 | 16.554 | −11.550 | 1.00 | 40.00 | ercc | C |
| ATOM | 642 | CD1 | LEU | A | 139 | 2.393 | 16.148 | −10.111 | 1.00 | 40.00 | ercc | C |
| ATOM | 646 | CD2 | LEU | A | 139 | 2.133 | 18.067 | −11.696 | 1.00 | 40.00 | ercc | C |
| ATOM | 650 | C | LEU | A | 139 | 2.594 | 13.752 | −13.692 | 1.00 | 40.00 | ercc | C |
| ATOM | 651 | O | LEU | A | 139 | 3.044 | 13.808 | −14.839 | 1.00 | 40.00 | ercc | O |
| ATOM | 652 | N | PHE | A | 140 | 1.520 | 13.087 | −13.399 | 1.00 | 40.00 | ercc | N |
| ATOM | 654 | CA | PHE | A | 140 | 0.774 | 12.382 | −14.455 | 1.00 | 40.00 | ercc | C |
| ATOM | 656 | CB | PHE | A | 140 | 0.468 | 10.986 | −13.942 | 1.00 | 40.00 | ercc | C |
| ATOM | 659 | CG | PHE | A | 140 | −0.286 | 10.189 | −15.000 | 1.00 | 40.00 | ercc | C |
| ATOM | 660 | CD1 | PHE | A | 140 | 0.208 | 8.951 | −15.417 | 1.00 | 40.00 | ercc | C |
| ATOM | 662 | CD2 | PHE | A | 140 | −1.476 | 10.666 | −15.546 | 1.00 | 40.00 | ercc | C |
| ATOM | 664 | CE1 | PHE | A | 140 | −0.484 | 8.199 | −16.368 | 1.00 | 40.00 | ercc | C |
| ATOM | 666 | CE2 | PHE | A | 140 | −2.164 | 9.915 | −16.499 | 1.00 | 40.00 | ercc | C |
| ATOM | 668 | CZ | PHE | A | 140 | −1.668 | 8.681 | −16.907 | 1.00 | 40.00 | ercc | C |
| ATOM | 670 | C | PHE | A | 140 | −0.506 | 13.092 | −14.685 | 1.00 | 40.00 | ercc | C |
| ATOM | 671 | O | PHE | A | 140 | −1.227 | 13.411 | −13.772 | 1.00 | 40.00 | ercc | O |
| ATOM | 672 | N | LEU | A | 141 | −0.810 | 13.298 | −15.898 | 1.00 | 40.00 | ercc | N |
| ATOM | 674 | CA | LEU | A | 141 | −2.062 | 13.946 | −16.221 | 1.00 | 40.00 | ercc | C |
| ATOM | 676 | CB | LEU | A | 141 | −1.702 | 15.445 | −16.364 | 1.00 | 40.00 | ercc | C |
| ATOM | 679 | CG | LEU | A | 141 | −1.784 | 15.942 | −17.811 | 1.00 | 40.00 | ercc | C |
| ATOM | 681 | CD1 | LEU | A | 141 | −3.231 | 16.272 | −18.149 | 1.00 | 40.00 | ercc | C |
| ATOM | 685 | CD2 | LEU | A | 141 | −0.936 | 17.207 | −17.971 | 1.00 | 40.00 | ercc | C |
| ATOM | 689 | C | LEU | A | 141 | −2.569 | 13.291 | −17.480 | 1.00 | 40.00 | ercc | C |
| ATOM | 690 | O | LEU | A | 141 | −2.074 | 13.525 | −18.551 | 1.00 | 40.00 | ercc | O |
| ATOM | 691 | N | SER | A | 142 | −3.563 | 12.455 | −17.393 | 1.00 | 40.00 | ercc | N |
| ATOM | 693 | CA | SER | A | 142 | −4.032 | 11.874 | −18.694 | 1.00 | 40.00 | ercc | C |
| ATOM | 695 | CB | SER | A | 142 | −5.142 | 10.877 | −18.414 | 1.00 | 40.00 | ercc | C |
| ATOM | 698 | OG | SER | A | 142 | −5.863 | 10.667 | −19.619 | 1.00 | 40.00 | ercc | O |
| ATOM | 700 | C | SER | A | 142 | −4.587 | 13.047 | −19.454 | 1.00 | 40.00 | ercc | C |
| ATOM | 701 | O | SER | A | 142 | −4.516 | 14.157 | −18.991 | 1.00 | 40.00 | ercc | O |
| ATOM | 702 | N | LEU | A | 143 | −5.160 | 12.884 | −20.564 | 1.00 | 40.00 | ercc | N |
| ATOM | 704 | CA | LEU | A | 143 | −5.691 | 14.103 | −21.186 | 1.00 | 40.00 | ercc | C |
| ATOM | 706 | CB | LEU | A | 143 | −5.065 | 14.227 | −22.558 | 1.00 | 40.00 | ercc | C |
| ATOM | 709 | CG | LEU | A | 143 | −4.593 | 15.671 | −22.732 | 1.00 | 40.00 | ercc | C |
| ATOM | 711 | CD1 | LEU | A | 143 | −3.672 | 15.776 | −23.953 | 1.00 | 40.00 | ercc | C |
| ATOM | 715 | CD2 | LEU | A | 143 | −5.818 | 16.574 | −22.916 | 1.00 | 40.00 | ercc | C |
| ATOM | 719 | C | LEU | A | 143 | −7.176 | 14.000 | −21.242 | 1.00 | 40.00 | ercc | C |
| ATOM | 720 | O | LEU | A | 143 | −7.869 | 14.979 | −21.063 | 1.00 | 40.00 | ercc | O |
| ATOM | 721 | N | ARG | A | 144 | −7.698 | 12.824 | −21.428 | 1.00 | 40.00 | ercc | N |
| ATOM | 723 | CA | ARG | A | 144 | −9.163 | 12.723 | −21.423 | 1.00 | 40.00 | ercc | C |
| ATOM | 725 | CB | ARG | A | 144 | −9.481 | 11.243 | −21.435 | 1.00 | 40.00 | ercc | C |
| ATOM | 728 | CG | ARG | A | 144 | −10.714 | 11.020 | −22.304 | 1.00 | 40.00 | ercc | C |
| ATOM | 731 | CD | ARG | A | 144 | −10.557 | 9.713 | −23.070 | 1.00 | 40.00 | ercc | C |
| ATOM | 734 | NE | ARG | A | 144 | −10.974 | 8.658 | −22.104 | 1.00 | 40.00 | ercc | N |
| ATOM | 736 | CZ | ARG | A | 144 | −10.941 | 7.392 | −22.443 | 1.00 | 40.00 | ercc | C |
| ATOM | 737 | NH1 | ARG | A | 144 | −10.392 | 7.016 | −23.575 | 1.00 | 40.00 | ercc | N |
| ATOM | 740 | NH2 | ARG | A | 144 | −11.458 | 6.496 | −21.641 | 1.00 | 40.00 | ercc | N |
| ATOM | 743 | C | ARG | A | 144 | −9.620 | 13.416 | −20.151 | 1.00 | 40.00 | ercc | C |
| ATOM | 744 | O | ARG | A | 144 | −10.354 | 14.377 | −20.203 | 1.00 | 40.00 | ercc | O |
| ATOM | 745 | N | TYR | A | 145 | −9.144 | 12.967 | −19.010 | 1.00 | 40.00 | ercc | N |
| ATOM | 747 | CA | TYR | A | 145 | −9.506 | 13.657 | −17.732 | 1.00 | 40.00 | ercc | C |
| ATOM | 749 | CB | TYR | A | 145 | −8.801 | 12.868 | −16.599 | 1.00 | 40.00 | ercc | C |
| ATOM | 752 | CG | TYR | A | 145 | −8.903 | 13.584 | −15.233 | 1.00 | 40.00 | ercc | C |
| ATOM | 753 | CD1 | TYR | A | 145 | −10.126 | 13.646 | −14.533 | 1.00 | 40.00 | ercc | C |
| ATOM | 755 | CD2 | TYR | A | 145 | −7.754 | 14.161 | −14.650 | 1.00 | 40.00 | ercc | C |
| ATOM | 757 | CE1 | TYR | A | 145 | −10.188 | 14.282 | −13.276 | 1.00 | 40.00 | ercc | C |
| ATOM | 759 | CE2 | TYR | A | 145 | −7.832 | 14.799 | −13.396 | 1.00 | 40.00 | ercc | C |
| ATOM | 761 | CZ | TYR | A | 145 | −9.046 | 14.856 | −12.714 | 1.00 | 40.00 | ercc | C |
| ATOM | 762 | OH | TYR | A | 145 | −9.118 | 15.482 | −11.492 | 1.00 | 40.00 | ercc | O |
| ATOM | 764 | C | TYR | A | 145 | −9.002 | 15.100 | −17.799 | 1.00 | 40.00 | ercc | C |
| ATOM | 765 | O | TYR | A | 145 | −9.602 | 15.994 | −17.221 | 1.00 | 40.00 | ercc | O |
| ATOM | 766 | N | HIS | A | 146 | −7.910 | 15.353 | −18.498 | 1.00 | 40.00 | ercc | N |
| ATOM | 768 | CA | HIS | A | 146 | −7.420 | 16.768 | −18.563 | 1.00 | 40.00 | ercc | C |
| ATOM | 770 | CB | HIS | A | 146 | −6.322 | 16.855 | −19.624 | 1.00 | 40.00 | ercc | C |
| ATOM | 773 | CG | HIS | A | 146 | −6.064 | 18.322 | −19.853 | 1.00 | 40.00 | ercc | C |
| ATOM | 774 | ND1 | HIS | A | 146 | −5.124 | 19.032 | −19.119 | 1.00 | 40.00 | ercc | N |
| ATOM | 776 | CD2 | HIS | A | 146 | −6.701 | 19.248 | −20.641 | 1.00 | 40.00 | ercc | C |
| ATOM | 778 | CE1 | HIS | A | 146 | −5.229 | 20.325 | −19.470 | 1.00 | 40.00 | ercc | C |
| ATOM | 780 | NE2 | HIS | A | 146 | −6.175 | 20.512 | −20.393 | 1.00 | 40.00 | ercc | N |
| ATOM | 781 | C | HIS | A | 146 | −8.570 | 17.696 | −18.962 | 1.00 | 40.00 | ercc | C |
| ATOM | 782 | O | HIS | A | 146 | −8.634 | 18.851 | −18.564 | 1.00 | 40.00 | ercc | O |
| ATOM | 783 | N | ASN | A | 147 | −9.466 | 17.198 | −19.755 | 1.00 | 40.00 | ercc | N |
| ATOM | 785 | CA | ASN | A | 147 | −10.605 | 18.036 | −20.200 | 1.00 | 40.00 | ercc | C |
| ATOM | 787 | CB | ASN | A | 147 | −11.311 | 17.200 | −21.261 | 1.00 | 40.00 | ercc | C |
| ATOM | 790 | CG | ASN | A | 147 | −10.944 | 17.746 | −22.637 | 1.00 | 40.00 | ercc | C |
| ATOM | 791 | OD1 | ASN | A | 147 | −11.792 | 18.288 | −23.336 | 1.00 | 40.00 | ercc | O |
| ATOM | 792 | ND2 | ASN | A | 147 | −9.701 | 17.640 | −23.053 | 1.00 | 40.00 | ercc | N |
| ATOM | 795 | C | ASN | A | 147 | −11.555 | 18.359 | −19.040 | 1.00 | 40.00 | ercc | C |
| ATOM | 796 | O | ASN | A | 147 | −12.005 | 19.470 | −18.901 | 1.00 | 40.00 | ercc | O |
| ATOM | 797 | N | LEU | A | 148 | −11.885 | 17.417 | −18.219 | 1.00 | 40.00 | ercc | N |
| ATOM | 799 | CA | LEU | A | 148 | −12.831 | 17.733 | −17.118 | 1.00 | 40.00 | ercc | C |
| ATOM | 801 | CB | LEU | A | 148 | −13.141 | 16.388 | −16.493 | 1.00 | 40.00 | ercc | C |
| ATOM | 804 | CG | LEU | A | 148 | −14.121 | 15.635 | −17.400 | 1.00 | 40.00 | ercc | C |
| ATOM | 806 | CD1 | LEU | A | 148 | −13.392 | 15.060 | −18.626 | 1.00 | 40.00 | ercc | C |
| ATOM | 810 | CD2 | LEU | A | 148 | −14.751 | 14.493 | −16.603 | 1.00 | 40.00 | ercc | C |
| ATOM | 814 | C | LEU | A | 148 | −12.209 | 18.703 | −16.112 | 1.00 | 40.00 | ercc | C |
| ATOM | 815 | O | LEU | A | 148 | −12.896 | 19.443 | −15.446 | 1.00 | 40.00 | ercc | O |
| ATOM | 816 | N | HIS | A | 149 | −10.925 | 18.719 | −15.995 | 1.00 | 40.00 | ercc | N |
| ATOM | 818 | CA | HIS | A | 149 | −10.297 | 19.653 | −15.033 | 1.00 | 40.00 | ercc | C |
| ATOM | 820 | CB | HIS | A | 149 | −9.878 | 18.813 | −13.828 | 1.00 | 40.00 | ercc | C |
| ATOM | 823 | CG | HIS | A | 149 | −10.913 | 17.778 | −13.471 | 1.00 | 40.00 | ercc | C |
| ATOM | 824 | ND1 | HIS | A | 149 | −11.319 | 16.789 | −14.356 | 1.00 | 40.00 | ercc | N |
| ATOM | 826 | CD2 | HIS | A | 149 | −11.596 | 17.541 | −12.301 | 1.00 | 40.00 | ercc | C |
| ATOM | 828 | CE1 | HIS | A | 149 | −12.208 | 16.011 | −13.710 | 1.00 | 40.00 | ercc | C |
| ATOM | 830 | NE2 | HIS | A | 149 | −12.410 | 16.423 | −12.451 | 1.00 | 40.00 | ercc | N |
| ATOM | 831 | C | HIS | A | 149 | −9.065 | 20.263 | −15.673 | 1.00 | 40.00 | ercc | C |
| ATOM | 832 | O | HIS | A | 149 | −7.995 | 19.732 | −15.566 | 1.00 | 40.00 | ercc | O |
| ATOM | 833 | N | PRO | A | 150 | −9.254 | 21.347 | −16.331 | 1.00 | 40.00 | ercc | N |
| ATOM | 834 | CA | PRO | A | 150 | −8.128 | 22.024 | −17.020 | 1.00 | 40.00 | ercc | C |
| ATOM | 836 | CB | PRO | A | 150 | −8.827 | 22.818 | −18.108 | 1.00 | 40.00 | ercc | C |
| ATOM | 839 | CG | PRO | A | 150 | −10.223 | 23.048 | −17.603 | 1.00 | 40.00 | ercc | C |
| ATOM | 842 | CD | PRO | A | 150 | −10.512 | 22.045 | −16.511 | 1.00 | 40.00 | ercc | C |
| ATOM | 845 | C | PRO | A | 150 | −7.305 | 22.949 | −16.074 | 1.00 | 40.00 | ercc | C |
| ATOM | 846 | O | PRO | A | 150 | −6.208 | 23.385 | −16.409 | 1.00 | 40.00 | ercc | O |
| ATOM | 847 | N | ASP | A | 151 | −7.780 | 23.237 | −14.898 | 1.00 | 40.00 | ercc | N |
| ATOM | 849 | CA | ASP | A | 151 | −6.961 | 24.085 | −13.980 | 1.00 | 40.00 | ercc | C |
| ATOM | 851 | CB | ASP | A | 151 | −7.991 | 24.941 | −13.225 | 1.00 | 40.00 | ercc | C |
| ATOM | 854 | CG | ASP | A | 151 | −7.285 | 26.043 | −12.409 | 1.00 | 40.00 | ercc | C |
| ATOM | 855 | OD1 | ASP | A | 151 | −6.181 | 25.789 | −11.912 | 1.00 | 40.00 | ercc | O |
| ATOM | 856 | OD2 | ASP | A | 151 | −7.864 | 27.124 | −12.288 | 1.00 | 40.00 | ercc | O |
| ATOM | 857 | C | ASP | A | 151 | −6.172 | 23.149 | −13.026 | 1.00 | 40.00 | ercc | C |
| ATOM | 858 | O | ASP | A | 151 | −5.192 | 23.528 | −12.398 | 1.00 | 40.00 | ercc | O |
| ATOM | 859 | N | TYR | A | 152 | −6.620 | 21.919 | −12.922 | 1.00 | 40.00 | ercc | N |
| ATOM | 861 | CA | TYR | A | 152 | −5.962 | 20.933 | −12.027 | 1.00 | 40.00 | ercc | C |
| ATOM | 863 | CB | TYR | A | 152 | −6.670 | 19.603 | −12.271 | 1.00 | 40.00 | ercc | C |
| ATOM | 866 | CG | TYR | A | 152 | −5.815 | 18.460 | −11.733 | 1.00 | 40.00 | ercc | C |
| ATOM | 867 | CD1 | TYR | A | 152 | −5.513 | 18.394 | −10.365 | 1.00 | 40.00 | ercc | C |
| ATOM | 869 | CD2 | TYR | A | 152 | −5.323 | 17.476 | −12.600 | 1.00 | 40.00 | ercc | C |
| ATOM | 871 | CE1 | TYR | A | 152 | −4.728 | 17.353 | −9.869 | 1.00 | 40.00 | ercc | C |
| ATOM | 873 | CE2 | TYR | A | 152 | −4.535 | 16.433 | −12.102 | 1.00 | 40.00 | ercc | C |
| ATOM | 875 | CZ | TYR | A | 152 | −4.238 | 16.372 | −10.737 | 1.00 | 40.00 | ercc | C |
| ATOM | 876 | OH | TYR | A | 152 | −3.459 | 15.347 | −10.245 | 1.00 | 40.00 | ercc | O |
| ATOM | 878 | C | TYR | A | 152 | −4.510 | 20.741 | −12.350 | 1.00 | 40.00 | ercc | C |
| ATOM | 879 | O | TYR | A | 152 | −3.690 | 20.779 | −11.463 | 1.00 | 40.00 | ercc | O |
| ATOM | 880 | N | ILE | A | 153 | −4.153 | 20.516 | −13.594 | 1.00 | 40.00 | ercc | N |
| ATOM | 882 | CA | ILE | A | 153 | −2.721 | 20.312 | −13.821 | 1.00 | 40.00 | ercc | C |
| ATOM | 884 | CB | ILE | A | 153 | −2.419 | 19.785 | −15.255 | 1.00 | 40.00 | ercc | C |
| ATOM | 886 | CG1 | ILE | A | 153 | −0.941 | 19.396 | −15.402 | 1.00 | 40.00 | ercc | C |
| ATOM | 889 | CG2 | ILE | A | 153 | −2.821 | 20.800 | −16.322 | 1.00 | 40.00 | ercc | C |
| ATOM | 893 | CD1 | ILE | A | 153 | −0.505 | 18.225 | −14.530 | 1.00 | 40.00 | ercc | C |
| ATOM | 897 | C | ILE | A | 153 | −1.907 | 21.567 | −13.525 | 1.00 | 40.00 | ercc | C |
| ATOM | 898 | O | ILE | A | 153 | −0.814 | 21.490 | −12.960 | 1.00 | 40.00 | ercc | O |
| ATOM | 899 | N | HIS | A | 154 | −2.456 | 22.715 | −13.908 | 1.00 | 40.00 | ercc | N |
| ATOM | 901 | CA | HIS | A | 154 | −1.828 | 23.997 | −13.649 | 1.00 | 40.00 | ercc | C |
| ATOM | 903 | CB | HIS | A | 154 | −2.647 | 25.122 | −14.274 | 1.00 | 40.00 | ercc | C |
| ATOM | 906 | CG | HIS | A | 154 | −2.121 | 26.483 | −13.960 | 1.00 | 40.00 | ercc | C |
| ATOM | 907 | ND1 | HIS | A | 154 | −1.024 | 27.019 | −14.597 | 1.00 | 40.00 | ercc | N |
| ATOM | 909 | CD2 | HIS | A | 154 | −2.525 | 27.410 | −13.059 | 1.00 | 40.00 | ercc | C |
| ATOM | 911 | CE1 | HIS | A | 154 | −0.780 | 28.222 | −14.111 | 1.00 | 40.00 | ercc | C |
| ATOM | 913 | NE2 | HIS | A | 154 | −1.676 | 28.483 | −13.177 | 1.00 | 40.00 | ercc | N |
| ATOM | 914 | C | HIS | A | 154 | −1.670 | 24.231 | −12.150 | 1.00 | 40.00 | ercc | C |
| ATOM | 915 | O | HIS | A | 154 | −0.572 | 24.536 | −11.673 | 1.00 | 40.00 | ercc | O |
| ATOM | 916 | N | GLY | A | 155 | −2.774 | 24.077 | −11.419 | 1.00 | 40.00 | ercc | N |
| ATOM | 918 | CA | GLY | A | 155 | −2.783 | 24.218 | −9.964 | 1.00 | 40.00 | ercc | C |
| ATOM | 921 | C | GLY | A | 155 | −1.980 | 23.139 | −9.260 | 1.00 | 40.00 | ercc | C |
| ATOM | 922 | O | GLY | A | 155 | −1.717 | 23.232 | −8.059 | 1.00 | 40.00 | ercc | O |
| ATOM | 923 | N | ARG | A | 156 | −1.593 | 22.115 | −10.017 | 1.00 | 40.00 | ercc | N |
| ATOM | 925 | CA | ARG | A | 156 | −0.778 | 21.030 | −9.495 | 1.00 | 40.00 | ercc | C |
| ATOM | 927 | CB | ARG | A | 156 | −1.041 | 19.743 | −10.285 | 1.00 | 40.00 | ercc | C |
| ATOM | 930 | CG | ARG | A | 156 | −0.527 | 18.476 | −9.627 | 1.00 | 40.00 | ercc | C |
| ATOM | 933 | CD | ARG | A | 156 | −1.132 | 18.242 | −8.255 | 1.00 | 40.00 | ercc | C |
| ATOM | 936 | NE | ARG | A | 156 | −0.372 | 17.240 | −7.513 | 1.00 | 40.00 | ercc | N |
| ATOM | 938 | CZ | ARG | A | 156 | −0.589 | 15.929 | −7.572 | 1.00 | 40.00 | ercc | C |
| ATOM | 939 | NH1 | ARG | A | 156 | −1.557 | 15.438 | −8.338 | 1.00 | 40.00 | ercc | N |
| ATOM | 942 | NH2 | ARG | A | 156 | 0.165 | 15.103 | −6.859 | 1.00 | 40.00 | ercc | N |
| ATOM | 945 | C | ARG | A | 156 | 0.709 | 21.390 | −9.500 | 1.00 | 40.00 | ercc | C |
| ATOM | 946 | O | ARG | A | 156 | 1.444 | 21.011 | −8.585 | 1.00 | 40.00 | ercc | O |
| ATOM | 947 | N | LEU | A | 157 | 1.145 | 22.118 | −10.526 | 1.00 | 40.00 | ercc | N |
| ATOM | 949 | CA | LEU | A | 157 | 2.539 | 22.554 | −10.621 | 1.00 | 40.00 | ercc | C |
| ATOM | 951 | CB | LEU | A | 157 | 2.892 | 22.977 | −12.050 | 1.00 | 40.00 | ercc | C |
| ATOM | 954 | CG | LEU | A | 157 | 3.458 | 21.891 | −12.972 | 1.00 | 40.00 | ercc | C |
| ATOM | 956 | CD1 | LEU | A | 157 | 3.279 | 22.276 | −14.431 | 1.00 | 40.00 | ercc | C |
| ATOM | 960 | CD2 | LEU | A | 157 | 4.926 | 21.597 | −12.665 | 1.00 | 40.00 | ercc | C |
| ATOM | 964 | C | LEU | A | 157 | 2.877 | 23.668 | −9.635 | 1.00 | 40.00 | ercc | C |
| ATOM | 965 | O | LEU | A | 157 | 3.962 | 23.669 | −9.050 | 1.00 | 40.00 | ercc | O |
| ATOM | 966 | N | GLN | A | 158 | 1.951 | 24.609 | −9.458 | 1.00 | 40.00 | ercc | N |
| ATOM | 968 | CA | GLN | A | 158 | 2.137 | 25.704 | −8.505 | 1.00 | 40.00 | ercc | C |
| ATOM | 970 | CB | GLN | A | 158 | 0.993 | 26.720 | −8.590 | 1.00 | 40.00 | ercc | C |
| ATOM | 973 | CG | GLN | A | 158 | 1.069 | 27.644 | −9.800 | 1.00 | 40.00 | ercc | C |
| ATOM | 976 | CD | GLN | A | 158 | 2.462 | 28.219 | −10.023 | 1.00 | 40.00 | ercc | C |
| ATOM | 977 | OE1 | GLN | A | 158 | 3.128 | 27.891 | −11.006 | 1.00 | 40.00 | ercc | O |
| ATOM | 978 | NE2 | GLN | A | 158 | 2.911 | 29.071 | −9.106 | 1.00 | 40.00 | ercc | N |
| ATOM | 981 | C | GLN | A | 158 | 2.281 | 25.184 | −7.082 | 1.00 | 40.00 | ercc | C |
| ATOM | 982 | O | GLN | A | 158 | 3.100 | 25.690 | −6.309 | 1.00 | 40.00 | ercc | O |
| ATOM | 983 | N | SER | A | 159 | 1.481 | 24.168 | −6.753 | 1.00 | 40.00 | ercc | N |
| ATOM | 985 | CA | SER | A | 159 | 1.568 | 23.484 | −5.467 | 1.00 | 40.00 | ercc | C |
| ATOM | 987 | CB | SER | A | 159 | 0.538 | 22.348 | −5.380 | 1.00 | 40.00 | ercc | C |
| ATOM | 990 | OG | SER | A | 159 | 0.938 | 21.216 | −6.136 | 1.00 | 40.00 | ercc | O |
| ATOM | 992 | C | SER | A | 159 | 2.976 | 22.938 | −5.256 | 1.00 | 40.00 | ercc | C |
| ATOM | 993 | O | SER | A | 159 | 3.578 | 23.146 | −4.199 | 1.00 | 40.00 | ercc | O |
| ATOM | 994 | N | LEU | A | 160 | 3.492 | 22.253 | −6.275 | 1.00 | 40.00 | ercc | N |
| ATOM | 996 | CA | LEU | A | 160 | 4.830 | 21.672 | −6.240 | 1.00 | 40.00 | ercc | C |
| ATOM | 998 | CB | LEU | A | 160 | 5.089 | 20.867 | −7.519 | 1.00 | 40.00 | ercc | C |
| ATOM | 1001 | CG | LEU | A | 160 | 6.390 | 20.071 | −7.662 | 1.00 | 40.00 | ercc | C |
| ATOM | 1003 | CD1 | LEU | A | 160 | 6.547 | 19.016 | −6.565 | 1.00 | 40.00 | ercc | C |
| ATOM | 1007 | CD2 | LEU | A | 160 | 6.441 | 19.429 | −9.037 | 1.00 | 40.00 | ercc | C |
| ATOM | 1011 | C | LEU | A | 160 | 5.911 | 22.732 | −6.038 | 1.00 | 40.00 | ercc | C |
| ATOM | 1012 | O | LEU | A | 160 | 6.929 | 22.473 | −5.387 | 1.00 | 40.00 | ercc | O |
| ATOM | 1013 | N | GLY | A | 161 | 5.675 | 23.919 | −6.591 | 1.00 | 40.00 | ercc | N |
| ATOM | 1015 | CA | GLY | A | 161 | 6.598 | 25.036 | −6.459 | 1.00 | 40.00 | ercc | C |
| ATOM | 1018 | C | GLY | A | 161 | 7.950 | 24.751 | −7.080 | 1.00 | 40.00 | ercc | C |
| ATOM | 1019 | O | GLY | A | 161 | 8.148 | 24.963 | −8.282 | 1.00 | 40.00 | ercc | O |
| ATOM | 1020 | N | LYS | A | 162 | 8.875 | 24.250 | −6.261 | 1.00 | 40.00 | ercc | N |
| ATOM | 1022 | CA | LYS | A | 162 | 10.282 | 24.144 | −6.650 | 1.00 | 40.00 | ercc | C |
| ATOM | 1024 | CB | LYS | A | 162 | 10.945 | 25.523 | −6.501 | 1.00 | 40.00 | ercc | C |
| ATOM | 1027 | CG | LYS | A | 162 | 12.135 | 25.792 | −7.418 | 1.00 | 40.00 | ercc | C |
| ATOM | 1030 | CD | LYS | A | 162 | 11.732 | 26.577 | −8.659 | 1.00 | 40.00 | ercc | C |
| ATOM | 1033 | CE | LYS | A | 162 | 12.953 | 27.194 | −9.331 | 1.00 | 40.00 | ercc | C |
| ATOM | 1036 | NZ | LYS | A | 162 | 12.587 | 27.994 | −10.534 | 1.00 | 40.00 | ercc | N |
| ATOM | 1040 | C | LYS | A | 162 | 11.078 | 23.098 | −5.849 | 1.00 | 40.00 | ercc | C |
| ATOM | 1041 | O | LYS | A | 162 | 12.303 | 23.016 | −5.979 | 1.00 | 40.00 | ercc | O |
| ATOM | 1042 | N | ASN | A | 163 | 10.390 | 22.293 | −5.040 | 1.00 | 40.00 | ercc | N |
| ATOM | 1044 | CA | ASN | A | 163 | 11.049 | 21.380 | −4.089 | 1.00 | 40.00 | ercc | C |
| ATOM | 1046 | CB | ASN | A | 163 | 10.013 | 20.762 | −3.149 | 1.00 | 40.00 | ercc | C |
| ATOM | 1049 | CG | ASN | A | 163 | 9.250 | 21.808 | −2.364 | 1.00 | 40.00 | ercc | C |
| ATOM | 1050 | OD1 | ASN | A | 163 | 9.772 | 22.394 | −1.415 | 1.00 | 40.00 | ercc | O |
| ATOM | 1051 | ND2 | ASN | A | 163 | 8.007 | 22.051 | −2.759 | 1.00 | 40.00 | ercc | N |
| ATOM | 1054 | C | ASN | A | 163 | 11.929 | 20.284 | −4.700 | 1.00 | 40.00 | ercc | C |
| ATOM | 1055 | O | ASN | A | 163 | 12.725 | 19.654 | −3.996 | 1.00 | 40.00 | ercc | O |
| ATOM | 1056 | N | PHE | A | 164 | 11.784 | 20.070 | −6.004 | 1.00 | 40.00 | ercc | N |
| ATOM | 1058 | CA | PHE | A | 164 | 12.520 | 19.030 | −6.716 | 1.00 | 40.00 | ercc | C |
| ATOM | 1060 | CB | PHE | A | 164 | 11.571 | 17.906 | −7.141 | 1.00 | 40.00 | ercc | C |
| ATOM | 1063 | CG | PHE | A | 164 | 11.057 | 17.073 | −5.996 | 1.00 | 40.00 | ercc | C |
| ATOM | 1064 | CD1 | PHE | A | 164 | 10.071 | 17.564 | −5.141 | 1.00 | 40.00 | ercc | C |
| ATOM | 1066 | CD2 | PHE | A | 164 | 11.549 | 15.790 | −5.781 | 1.00 | 40.00 | ercc | C |
| ATOM | 1068 | CE1 | PHE | A | 164 | 9.596 | 16.797 | −4.082 | 1.00 | 40.00 | ercc | C |
| ATOM | 1070 | CE2 | PHE | A | 164 | 11.078 | 15.012 | −4.725 | 1.00 | 40.00 | ercc | C |
| ATOM | 1072 | CZ | PHE | A | 164 | 10.099 | 15.518 | −3.874 | 1.00 | 40.00 | ercc | C |
| ATOM | 1074 | C | PHE | A | 164 | 13.240 | 19.605 | −7.933 | 1.00 | 40.00 | ercc | C |
| ATOM | 1075 | O | PHE | A | 164 | 12.639 | 20.328 | −8.733 | 1.00 | 40.00 | ercc | O |
| ATOM | 1076 | N | ALA | A | 165 | 14.526 | 19.285 | −8.059 | 1.00 | 40.00 | ercc | N |
| ATOM | 1078 | CA | ALA | A | 165 | 15.349 | 19.736 | −9.183 | 1.00 | 40.00 | ercc | C |
| ATOM | 1080 | CB | ALA | A | 165 | 16.807 | 19.319 | −8.995 | 1.00 | 40.00 | ercc | C |
| ATOM | 1084 | C | ALA | A | 165 | 14.810 | 19.234 | −10.521 | 1.00 | 40.00 | ercc | C |
| ATOM | 1085 | O | ALA | A | 165 | 14.439 | 20.040 | −11.375 | 1.00 | 40.00 | ercc | O |
| ATOM | 1086 | N | LEU | A | 166 | 14.760 | 17.916 | −10.706 | 1.00 | 40.00 | ercc | N |
| ATOM | 1088 | CA | LEU | A | 166 | 14.241 | 17.367 | −11.952 | 1.00 | 40.00 | ercc | C |
| ATOM | 1090 | CB | LEU | A | 166 | 14.916 | 16.039 | −12.315 | 1.00 | 40.00 | ercc | C |
| ATOM | 1093 | CG | LEU | A | 166 | 15.361 | 15.838 | −13.774 | 1.00 | 40.00 | ercc | C |
| ATOM | 1095 | CD1 | LEU | A | 166 | 15.687 | 14.372 | −14.033 | 1.00 | 40.00 | ercc | C |
| ATOM | 1099 | CD2 | LEU | A | 166 | 14.340 | 16.350 | −14.801 | 1.00 | 40.00 | ercc | C |
| ATOM | 1103 | C | LEU | A | 166 | 12.726 | 17.222 | −11.895 | 1.00 | 40.00 | ercc | C |
| ATOM | 1104 | O | LEU | A | 166 | 12.191 | 16.354 | −11.204 | 1.00 | 40.00 | ercc | O |
| ATOM | 1105 | N | ARG | A | 167 | 12.041 | 18.090 | −12.629 | 1.00 | 40.00 | ercc | N |
| ATOM | 1107 | CA | ARG | A | 167 | 10.589 | 18.050 | −12.697 | 1.00 | 40.00 | ercc | C |
| ATOM | 1109 | CB | ARG | A | 167 | 9.988 | 19.398 | −12.291 | 1.00 | 40.00 | ercc | C |
| ATOM | 1112 | CG | ARG | A | 167 | 10.135 | 19.675 | −10.791 | 1.00 | 40.00 | ercc | C |
| ATOM | 1115 | CD | ARG | A | 167 | 9.678 | 21.067 | −10.378 | 1.00 | 40.00 | ercc | C |
| ATOM | 1118 | NE | ARG | A | 167 | 10.661 | 22.101 | −10.696 | 1.00 | 40.00 | ercc | N |
| ATOM | 1120 | CZ | ARG | A | 167 | 10.644 | 22.840 | −11.802 | 1.00 | 40.00 | ercc | C |
| ATOM | 1121 | NH1 | ARG | A | 167 | 9.692 | 22.663 | −12.708 | 1.00 | 40.00 | ercc | N |
| ATOM | 1124 | NH2 | ARG | A | 167 | 11.581 | 23.757 | −12.004 | 1.00 | 40.00 | ercc | N |
| ATOM | 1127 | C | ARG | A | 167 | 10.148 | 17.584 | −14.078 | 1.00 | 40.00 | ercc | C |
| ATOM | 1128 | O | ARG | A | 167 | 10.642 | 18.066 | −15.097 | 1.00 | 40.00 | ercc | O |
| ATOM | 1129 | N | VAL | A | 168 | 9.236 | 16.614 | −14.086 | 1.00 | 40.00 | ercc | N |
| ATOM | 1131 | CA | VAL | A | 168 | 8.910 | 15.842 | −15.282 | 1.00 | 40.00 | ercc | C |
| ATOM | 1133 | CB | VAL | A | 168 | 9.593 | 14.452 | −15.250 | 1.00 | 40.00 | ercc | C |
| ATOM | 1135 | CG1 | VAL | A | 168 | 9.296 | 13.699 | −16.519 | 1.00 | 40.00 | ercc | C |
| ATOM | 1139 | CG2 | VAL | A | 168 | 11.104 | 14.572 | −15.048 | 1.00 | 40.00 | ercc | C |
| ATOM | 1143 | C | VAL | A | 168 | 7.403 | 15.621 | −15.416 | 1.00 | 40.00 | ercc | C |
| ATOM | 1144 | O | VAL | A | 168 | 6.738 | 15.243 | −14.450 | 1.00 | 40.00 | ercc | O |
| ATOM | 1145 | N | LEU | A | 169 | 6.867 | 15.848 | −16.614 | 1.00 | 40.00 | ercc | N |
| ATOM | 1147 | CA | LEU | A | 169 | 5.429 | 15.719 | −16.848 | 1.00 | 40.00 | ercc | C |
| ATOM | 1149 | CB | LEU | A | 169 | 4.872 | 16.996 | −17.491 | 1.00 | 40.00 | ercc | C |
| ATOM | 1152 | CG | LEU | A | 169 | 3.402 | 17.405 | −17.300 | 1.00 | 40.00 | ercc | C |
| ATOM | 1154 | CD1 | LEU | A | 169 | 2.919 | 18.163 | −18.529 | 1.00 | 40.00 | ercc | C |
| ATOM | 1158 | CD2 | LEU | A | 169 | 2.452 | 16.240 | −17.015 | 1.00 | 40.00 | ercc | C |
| ATOM | 1162 | C | LEU | A | 169 | 5.087 | 14.505 | −17.714 | 1.00 | 40.00 | ercc | C |
| ATOM | 1163 | O | LEU | A | 169 | 5.236 | 14.537 | −18.939 | 1.00 | 40.00 | ercc | O |
| ATOM | 1164 | N | LEU | A | 170 | 4.612 | 13.444 | −17.067 | 1.00 | 40.00 | ercc | N |
| ATOM | 1166 | CA | LEU | A | 170 | 4.197 | 12.229 | −17.763 | 1.00 | 40.00 | ercc | C |
| ATOM | 1168 | CB | LEU | A | 170 | 4.267 | 11.014 | −16.828 | 1.00 | 40.00 | ercc | C |
| ATOM | 1171 | CG | LEU | A | 170 | 3.948 | 9.616 | −17.389 | 1.00 | 40.00 | ercc | C |
| ATOM | 1173 | CD1 | LEU | A | 170 | 4.754 | 9.279 | −18.643 | 1.00 | 40.00 | ercc | C |
| ATOM | 1177 | CD2 | LEU | A | 170 | 4.168 | 8.562 | −16.320 | 1.00 | 40.00 | ercc | C |
| ATOM | 1181 | C | LEU | A | 170 | 2.796 | 12.394 | −18.349 | 1.00 | 40.00 | ercc | C |
| ATOM | 1182 | O | LEU | A | 170 | 1.831 | 12.638 | −17.620 | 1.00 | 40.00 | ercc | O |
| ATOM | 1183 | N | VAL | A | 171 | 2.700 | 12.263 | −19.670 | 1.00 | 40.00 | ercc | N |
| ATOM | 1185 | CA | VAL | A | 171 | 1.455 | 12.528 | −20.392 | 1.00 | 40.00 | ercc | C |
| ATOM | 1187 | CB | VAL | A | 171 | 1.619 | 13.712 | −21.389 | 1.00 | 40.00 | ercc | C |
| ATOM | 1189 | CG1 | VAL | A | 171 | 0.316 | 13.990 | −22.130 | 1.00 | 40.00 | ercc | C |
| ATOM | 1193 | CG2 | VAL | A | 171 | 2.089 | 14.970 | −20.659 | 1.00 | 40.00 | ercc | C |
| ATOM | 1197 | C | VAL | A | 171 | 0.958 | 11.291 | −21.137 | 1.00 | 40.00 | ercc | C |
| ATOM | 1198 | O | VAL | A | 171 | 1.653 | 10.762 | −22.007 | 1.00 | 40.00 | ercc | O |
| ATOM | 1199 | N | GLN | A | 172 | −0.244 | 10.833 | −20.790 | 1.00 | 40.00 | ercc | N |
| ATOM | 1201 | CA | GLN | A | 172 | −0.874 | 9.729 | −21.511 | 1.00 | 40.00 | ercc | C |
| ATOM | 1203 | CB | GLN | A | 172 | −1.683 | 8.814 | −20.582 | 1.00 | 40.00 | ercc | C |
| ATOM | 1206 | CG | GLN | A | 172 | −2.283 | 7.602 | −21.305 | 1.00 | 40.00 | ercc | C |
| ATOM | 1209 | CD | GLN | A | 172 | −2.998 | 6.634 | −20.379 | 1.00 | 40.00 | ercc | C |
| ATOM | 1210 | OE1 | GLN | A | 172 | −2.396 | 6.068 | −19.466 | 1.00 | 40.00 | ercc | O |
| ATOM | 1211 | NE2 | GLN | A | 172 | −4.288 | 6.428 | −20.622 | 1.00 | 40.00 | ercc | N |
| ATOM | 1214 | C | GLN | A | 172 | −1.742 | 10.247 | −22.655 | 1.00 | 40.00 | ercc | C |
| ATOM | 1215 | O | GLN | A | 172 | −2.813 | 10.821 | −22.437 | 1.00 | 40.00 | ercc | O |
| ATOM | 1216 | N | VAL | A | 173 | −1.255 | 10.032 | −23.875 | 1.00 | 40.00 | ercc | N |
| ATOM | 1218 | CA | VAL | A | 173 | −1.967 | 10.407 | −25.094 | 1.00 | 40.00 | ercc | C |
| ATOM | 1220 | CB | VAL | A | 173 | −1.015 | 10.521 | −26.325 | 1.00 | 40.00 | ercc | C |
| ATOM | 1222 | CG1 | VAL | A | 173 | −1.719 | 11.243 | −27.471 | 1.00 | 40.00 | ercc | C |
| ATOM | 1226 | CG2 | VAL | A | 173 | 0.266 | 11.280 | −25.964 | 1.00 | 40.00 | ercc | C |
| ATOM | 1230 | C | VAL | A | 173 | −3.071 | 9.381 | −25.358 | 1.00 | 40.00 | ercc | C |
| ATOM | 1231 | O | VAL | A | 173 | −2.923 | 8.476 | −26.186 | 1.00 | 40.00 | ercc | O |
| ATOM | 1232 | N | ASP | A | 174 | −4.173 | 9.531 | −24.632 | 1.00 | 40.00 | ercc | N |
| ATOM | 1234 | CA | ASP | A | 174 | −5.301 | 8.611 | −24.732 | 1.00 | 40.00 | ercc | C |
| ATOM | 1236 | CB | ASP | A | 174 | −5.709 | 8.113 | −23.336 | 1.00 | 40.00 | ercc | C |
| ATOM | 1239 | CG | ASP | A | 174 | −6.067 | 9.245 | −22.375 | 1.00 | 40.00 | ercc | C |
| ATOM | 1240 | OD1 | ASP | A | 174 | −5.878 | 10.438 | −22.713 | 1.00 | 40.00 | ercc | O |
| ATOM | 1241 | OD2 | ASP | A | 174 | −6.542 | 8.928 | −21.263 | 1.00 | 40.00 | ercc | O |
| ATOM | 1242 | C | ASP | A | 174 | −6.486 | 9.248 | −25.460 | 1.00 | 40.00 | ercc | C |
| ATOM | 1243 | O | ASP | A | 174 | −7.643 | 8.877 | −25.231 | 1.00 | 40.00 | ercc | O |
| ATOM | 1244 | N | VAL | A | 175 | −6.184 | 10.198 | −26.347 | 1.00 | 40.00 | ercc | N |
| ATOM | 1246 | CA | VAL | A | 175 | −7.200 | 11.016 | −27.017 | 1.00 | 40.00 | ercc | C |
| ATOM | 1248 | CB | VAL | A | 175 | −7.470 | 12.336 | −26.228 | 1.00 | 40.00 | ercc | C |
| ATOM | 1250 | CG1 | VAL | A | 175 | −8.385 | 13.279 | −27.007 | 1.00 | 40.00 | ercc | C |
| ATOM | 1254 | CG2 | VAL | A | 175 | −8.069 | 12.047 | −24.859 | 1.00 | 40.00 | ercc | C |
| ATOM | 1258 | C | VAL | A | 175 | −6.772 | 11.349 | −28.451 | 1.00 | 40.00 | ercc | C |
| ATOM | 1259 | O | VAL | A | 175 | −5.600 | 11.635 | −28.702 | 1.00 | 40.00 | ercc | O |
| ATOM | 1260 | N | LYS | A | 176 | −7.726 | 11.311 | −29.383 | 1.00 | 40.00 | ercc | N |
| ATOM | 1262 | CA | LYS | A | 176 | −7.474 | 11.655 | −30.788 | 1.00 | 40.00 | ercc | C |
| ATOM | 1264 | CB | LYS | A | 176 | −8.468 | 10.929 | −31.705 | 1.00 | 40.00 | ercc | C |
| ATOM | 1267 | CG | LYS | A | 176 | −8.023 | 10.788 | −33.162 | 1.00 | 40.00 | ercc | C |
| ATOM | 1270 | CD | LYS | A | 176 | −8.887 | 9.772 | −33.903 | 1.00 | 40.00 | ercc | C |
| ATOM | 1273 | CE | LYS | A | 176 | −8.410 | 9.548 | −35.334 | 1.00 | 40.00 | ercc | C |
| ATOM | 1276 | NZ | LYS | A | 176 | −8.805 | 10.663 | −36.252 | 1.00 | 40.00 | ercc | N |
| ATOM | 1280 | C | LYS | A | 176 | −7.534 | 13.171 | −31.014 | 1.00 | 40.00 | ercc | C |
| ATOM | 1281 | O | LYS | A | 176 | −8.371 | 13.860 | −30.423 | 1.00 | 40.00 | ercc | O |
| ATOM | 1282 | N | ASP | A | 177 | −6.644 | 13.667 | −31.876 | 1.00 | 40.00 | ercc | N |
| ATOM | 1284 | CA | ASP | A | 177 | −6.455 | 15.107 | −32.139 | 1.00 | 40.00 | ercc | C |
| ATOM | 1286 | CB | ASP | A | 177 | −7.759 | 15.770 | −32.622 | 1.00 | 40.00 | ercc | C |
| ATOM | 1289 | CG | ASP | A | 177 | −8.171 | 15.320 | −34.015 | 1.00 | 40.00 | ercc | C |
| ATOM | 1290 | OD1 | ASP | A | 177 | −9.307 | 14.821 | −34.162 | 1.00 | 40.00 | ercc | O |
| ATOM | 1291 | OD2 | ASP | A | 177 | −7.368 | 15.467 | −34.963 | 1.00 | 40.00 | ercc | O |
| ATOM | 1292 | C | ASP | A | 177 | −5.860 | 15.887 | −30.946 | 1.00 | 40.00 | ercc | C |
| ATOM | 1293 | O | ASP | A | 177 | −6.296 | 17.010 | −30.663 | 1.00 | 40.00 | ercc | O |
| ATOM | 1294 | N | PRO | A | 178 | −4.844 | 15.309 | −30.261 | 1.00 | 40.00 | ercc | N |
| ATOM | 1295 | CA | PRO | A | 178 | −4.322 | 15.912 | −29.026 | 1.00 | 40.00 | ercc | C |
| ATOM | 1297 | CB | PRO | A | 178 | −3.348 | 14.851 | −28.505 | 1.00 | 40.00 | ercc | C |
| ATOM | 1300 | CG | PRO | A | 178 | −2.927 | 14.096 | −29.706 | 1.00 | 40.00 | ercc | C |
| ATOM | 1303 | CD | PRO | A | 178 | −4.120 | 14.070 | −30.608 | 1.00 | 40.00 | ercc | C |
| ATOM | 1306 | C | PRO | A | 178 | −3.580 | 17.229 | −29.235 | 1.00 | 40.00 | ercc | C |
| ATOM | 1307 | O | PRO | A | 178 | −3.594 | 18.088 | −28.350 | 1.00 | 40.00 | ercc | O |
| ATOM | 1308 | N | GLN | A | 179 | −2.952 | 17.372 | −30.403 | 1.00 | 40.00 | ercc | N |
| ATOM | 1310 | CA | GLN | A | 179 | −2.089 | 18.511 | −30.746 | 1.00 | 40.00 | ercc | C |
| ATOM | 1312 | CB | GLN | A | 179 | −2.115 | 18.793 | −32.262 | 1.00 | 40.00 | ercc | C |
| ATOM | 1315 | CG | GLN | A | 179 | −3.489 | 19.147 | −32.866 | 1.00 | 40.00 | ercc | C |
| ATOM | 1318 | CD | GLN | A | 179 | −4.295 | 17.934 | −33.314 | 1.00 | 40.00 | ercc | C |
| ATOM | 1319 | OE1 | GLN | A | 179 | −3.870 | 16.789 | −33.146 | 1.00 | 40.00 | ercc | O |
| ATOM | 1320 | NE2 | GLN | A | 179 | −5.462 | 18.187 | −33.899 | 1.00 | 40.00 | ercc | N |
| ATOM | 1323 | C | GLN | A | 179 | −2.370 | 19.784 | −29.949 | 1.00 | 40.00 | ercc | C |
| ATOM | 1324 | O | GLN | A | 179 | −1.495 | 20.281 | −29.233 | 1.00 | 40.00 | ercc | O |
| ATOM | 1325 | N | GLN | A | 180 | −3.598 | 20.284 | −30.072 | 1.00 | 40.00 | ercc | N |
| ATOM | 1327 | CA | GLN | A | 180 | −4.024 | 21.538 | −29.459 | 1.00 | 40.00 | ercc | C |
| ATOM | 1329 | CB | GLN | A | 180 | −5.534 | 21.729 | −29.621 | 1.00 | 40.00 | ercc | C |
| ATOM | 1332 | CG | GLN | A | 180 | −5.920 | 22.779 | −30.651 | 1.00 | 40.00 | ercc | C |
| ATOM | 1335 | CD | GLN | A | 180 | −5.910 | 24.193 | −30.086 | 1.00 | 40.00 | ercc | C |
| ATOM | 1336 | OE1 | GLN | A | 180 | −6.929 | 24.884 | −30.107 | 1.00 | 40.00 | ercc | O |
| ATOM | 1337 | NE2 | GLN | A | 180 | −4.759 | 24.629 | −29.578 | 1.00 | 40.00 | ercc | N |
| ATOM | 1340 | C | GLN | A | 180 | −3.633 | 21.672 | −27.993 | 1.00 | 40.00 | ercc | C |
| ATOM | 1341 | O | GLN | A | 180 | −2.964 | 22.635 | −27.613 | 1.00 | 40.00 | ercc | O |
| ATOM | 1342 | N | ALA | A | 181 | −4.057 | 20.706 | −27.180 | 1.00 | 40.00 | ercc | N |
| ATOM | 1344 | CA | ALA | A | 181 | −3.750 | 20.706 | −25.756 | 1.00 | 40.00 | ercc | C |
| ATOM | 1346 | CB | ALA | A | 181 | −4.372 | 19.490 | −25.077 | 1.00 | 40.00 | ercc | C |
| ATOM | 1350 | C | ALA | A | 181 | −2.243 | 20.743 | −25.521 | 1.00 | 40.00 | ercc | C |
| ATOM | 1351 | O | ALA | A | 181 | −1.735 | 21.623 | −24.820 | 1.00 | 40.00 | ercc | O |
| ATOM | 1352 | N | LEU | A | 182 | −1.542 | 19.792 | −26.137 | 1.00 | 40.00 | ercc | N |
| ATOM | 1354 | CA | LEU | A | 182 | −0.095 | 19.628 | −25.984 | 1.00 | 40.00 | ercc | C |
| ATOM | 1356 | CB | LEU | A | 182 | 0.442 | 18.620 | −27.008 | 1.00 | 40.00 | ercc | C |
| ATOM | 1359 | CG | LEU | A | 182 | −0.429 | 17.408 | −27.374 | 1.00 | 40.00 | ercc | C |
| ATOM | 1361 | CD1 | LEU | A | 182 | 0.188 | 16.638 | −28.532 | 1.00 | 40.00 | ercc | C |
| ATOM | 1365 | CD2 | LEU | A | 182 | −0.642 | 16.478 | −26.183 | 1.00 | 40.00 | ercc | C |
| ATOM | 1369 | C | LEU | A | 182 | 0.630 | 20.959 | −26.129 | 1.00 | 40.00 | ercc | C |
| ATOM | 1370 | O | LEU | A | 182 | 1.489 | 21.296 | −25.314 | 1.00 | 40.00 | ercc | O |
| ATOM | 1371 | N | LYS | A | 183 | 0.263 | 21.706 | −27.170 | 1.00 | 40.00 | ercc | N |
| ATOM | 1373 | CA | LYS | A | 183 | 0.762 | 23.056 | −27.402 | 1.00 | 40.00 | ercc | C |
| ATOM | 1375 | CB | LYS | A | 183 | −0.040 | 23.717 | −28.530 | 1.00 | 40.00 | ercc | C |
| ATOM | 1378 | CG | LYS | A | 183 | 0.478 | 25.068 | −28.999 | 1.00 | 40.00 | ercc | C |
| ATOM | 1381 | CD | LYS | A | 183 | −0.465 | 25.670 | −30.032 | 1.00 | 40.00 | ercc | C |
| ATOM | 1384 | CE | LYS | A | 183 | 0.228 | 26.746 | −30.857 | 1.00 | 40.00 | ercc | C |
| ATOM | 1387 | NZ | LYS | A | 183 | −0.601 | 27.184 | −32.019 | 1.00 | 40.00 | ercc | N |
| ATOM | 1391 | C | LYS | A | 183 | 0.700 | 23.902 | −26.128 | 1.00 | 40.00 | ercc | C |
| ATOM | 1392 | O | LYS | A | 183 | 1.734 | 24.364 | −25.639 | 1.00 | 40.00 | ercc | O |
| ATOM | 1393 | N | GLU | A | 184 | −0.506 | 24.081 | −25.587 | 1.00 | 40.00 | ercc | N |
| ATOM | 1395 | CA | GLU | A | 184 | −0.724 | 24.935 | −24.414 | 1.00 | 40.00 | ercc | C |
| ATOM | 1397 | CB | GLU | A | 184 | −2.220 | 25.215 | −24.213 | 1.00 | 40.00 | ercc | C |
| ATOM | 1400 | CG | GLU | A | 184 | −2.978 | 24.133 | −23.441 | 1.00 | 40.00 | ercc | C |
| ATOM | 1403 | CD | GLU | A | 184 | −4.486 | 24.253 | −23.560 | 1.00 | 40.00 | ercc | C |
| ATOM | 1404 | OE1 | GLU | A | 184 | −4.982 | 24.611 | −24.651 | 1.00 | 40.00 | ercc | O |
| ATOM | 1405 | OE2 | GLU | A | 184 | −5.180 | 23.976 | −22.559 | 1.00 | 40.00 | ercc | O |
| ATOM | 1406 | C | GLU | A | 184 | −0.109 | 24.356 | −23.137 | 1.00 | 40.00 | ercc | C |
| ATOM | 1407 | O | GLU | A | 184 | 0.192 | 25.093 | −22.193 | 1.00 | 40.00 | ercc | O |
| ATOM | 1408 | N | LEU | A | 185 | 0.067 | 23.036 | −23.120 | 1.00 | 40.00 | ercc | N |
| ATOM | 1410 | CA | LEU | A | 185 | 0.693 | 22.342 | −21.999 | 1.00 | 40.00 | ercc | C |
| ATOM | 1412 | CB | LEU | A | 185 | 0.359 | 20.850 | −22.022 | 1.00 | 40.00 | ercc | C |
| ATOM | 1415 | CG | LEU | A | 185 | −1.012 | 20.387 | −21.524 | 1.00 | 40.00 | ercc | C |
| ATOM | 1417 | CD1 | LEU | A | 185 | −1.155 | 18.874 | −21.703 | 1.00 | 40.00 | ercc | C |
| ATOM | 1421 | CD2 | LEU | A | 185 | −1.266 | 20.791 | −20.070 | 1.00 | 40.00 | ercc | C |
| ATOM | 1425 | C | LEU | A | 185 | 2.199 | 22.515 | −22.030 | 1.00 | 40.00 | ercc | C |
| ATOM | 1426 | O | LEU | A | 185 | 2.821 | 22.758 | −20.996 | 1.00 | 40.00 | ercc | O |
| ATOM | 1427 | N | ALA | A | 186 | 2.779 | 22.363 | −23.220 | 1.00 | 40.00 | ercc | N |
| ATOM | 1429 | CA | ALA | A | 186 | 4.204 | 22.584 | −23.429 | 1.00 | 40.00 | ercc | C |
| ATOM | 1431 | CB | ALA | A | 186 | 4.566 | 22.410 | −24.898 | 1.00 | 40.00 | ercc | C |
| ATOM | 1435 | C | ALA | A | 186 | 4.582 | 23.975 | −22.948 | 1.00 | 40.00 | ercc | C |
| ATOM | 1436 | O | ALA | A | 186 | 5.537 | 24.132 | −22.186 | 1.00 | 40.00 | ercc | O |
| ATOM | 1437 | N | LYS | A | 187 | 3.816 | 24.973 | −23.389 | 1.00 | 40.00 | ercc | N |
| ATOM | 1439 | CA | LYS | A | 187 | 4.009 | 26.362 | −22.973 | 1.00 | 40.00 | ercc | C |
| ATOM | 1441 | CB | LYS | A | 187 | 2.883 | 27.253 | −23.507 | 1.00 | 40.00 | ercc | C |
| ATOM | 1444 | CG | LYS | A | 187 | 2.933 | 27.458 | −25.014 | 1.00 | 40.00 | ercc | C |
| ATOM | 1447 | CD | LYS | A | 187 | 1.866 | 28.424 | −25.502 | 1.00 | 40.00 | ercc | C |
| ATOM | 1450 | CE | LYS | A | 187 | 1.992 | 28.655 | −27.003 | 1.00 | 40.00 | ercc | C |
| ATOM | 1453 | NZ | LYS | A | 187 | 1.071 | 29.719 | −27.493 | 1.00 | 40.00 | ercc | N |
| ATOM | 1457 | C | LYS | A | 187 | 4.108 | 26.463 | −21.456 | 1.00 | 40.00 | ercc | C |
| ATOM | 1458 | O | LYS | A | 187 | 5.037 | 27.076 | −20.932 | 1.00 | 40.00 | ercc | O |
| ATOM | 1459 | N | MET | A | 188 | 3.157 | 25.832 | −20.769 | 1.00 | 40.00 | ercc | N |
| ATOM | 1461 | CA | MET | A | 188 | 3.143 | 25.747 | −19.312 | 1.00 | 40.00 | ercc | C |
| ATOM | 1463 | CB | MET | A | 188 | 1.992 | 24.838 | −18.864 | 1.00 | 40.00 | ercc | C |
| ATOM | 1466 | CG | MET | A | 188 | 1.416 | 25.151 | −17.492 | 1.00 | 40.00 | ercc | C |
| ATOM | 1469 | SD | MET | A | 188 | −0.082 | 24.202 | −17.133 | 1.00 | 40.00 | ercc | S |
| ATOM | 1470 | CE | MET | A | 188 | −1.243 | 24.903 | −18.309 | 1.00 | 40.00 | ercc | C |
| ATOM | 1474 | C | MET | A | 188 | 4.474 | 25.223 | −18.767 | 1.00 | 40.00 | ercc | C |
| ATOM | 1475 | O | MET | A | 188 | 5.022 | 25.779 | −17.816 | 1.00 | 40.00 | ercc | O |
| ATOM | 1476 | N | CYS | A | 189 | 4.993 | 24.171 | −19.402 | 1.00 | 40.00 | ercc | N |
| ATOM | 1478 | CA | CYS | A | 189 | 6.172 | 23.445 | −18.924 | 1.00 | 40.00 | ercc | C |
| ATOM | 1480 | CB | CYS | A | 189 | 6.296 | 22.105 | −19.648 | 1.00 | 40.00 | ercc | C |
| ATOM | 1483 | SG | CYS | A | 189 | 4.940 | 20.964 | −19.340 | 1.00 | 40.00 | ercc | S |
| ATOM | 1485 | C | CYS | A | 189 | 7.495 | 24.195 | −19.038 | 1.00 | 40.00 | ercc | C |
| ATOM | 1486 | O | CYS | A | 189 | 8.394 | 23.957 | −18.238 | 1.00 | 40.00 | ercc | O |
| ATOM | 1487 | N | ILE | A | 190 | 7.625 | 25.062 | −20.042 | 1.00 | 40.00 | ercc | N |
| ATOM | 1489 | CA | ILE | A | 190 | 8.840 | 25.865 | −20.216 | 1.00 | 40.00 | ercc | C |
| ATOM | 1491 | CB | ILE | A | 190 | 8.842 | 26.685 | −21.531 | 1.00 | 40.00 | ercc | C |
| ATOM | 1493 | CG1 | ILE | A | 190 | 8.109 | 25.945 | −22.658 | 1.00 | 40.00 | ercc | C |
| ATOM | 1496 | CG2 | ILE | A | 190 | 10.278 | 27.045 | −21.930 | 1.00 | 40.00 | ercc | C |
| ATOM | 1500 | CD1 | ILE | A | 190 | 7.710 | 26.846 | −23.834 | 1.00 | 40.00 | ercc | C |
| ATOM | 1504 | C | ILE | A | 190 | 8.932 | 26.848 | −19.063 | 1.00 | 40.00 | ercc | C |
| ATOM | 1505 | O | ILE | A | 190 | 9.972 | 26.974 | −18.416 | 1.00 | 40.00 | ercc | O |
| ATOM | 1506 | N | LEU | A | 191 | 7.819 | 27.541 | −18.830 | 1.00 | 40.00 | ercc | N |
| ATOM | 1508 | CA | LEU | A | 191 | 7.669 | 28.463 | −17.713 | 1.00 | 40.00 | ercc | C |
| ATOM | 1510 | CB | LEU | A | 191 | 6.285 | 29.126 | −17.742 | 1.00 | 40.00 | ercc | C |
| ATOM | 1513 | CG | LEU | A | 191 | 5.588 | 29.380 | −19.089 | 1.00 | 40.00 | ercc | C |
| ATOM | 1515 | CD1 | LEU | A | 191 | 4.122 | 29.749 | −18.879 | 1.00 | 40.00 | ercc | C |
| ATOM | 1519 | CD2 | LEU | A | 191 | 6.299 | 30.438 | −19.930 | 1.00 | 40.00 | ercc | C |
| ATOM | 1523 | C | LEU | A | 191 | 7.855 | 27.691 | −16.412 | 1.00 | 40.00 | ercc | C |
| ATOM | 1524 | O | LEU | A | 191 | 8.545 | 28.153 | −15.501 | 1.00 | 40.00 | ercc | O |
| ATOM | 1525 | N | ALA | A | 192 | 7.243 | 26.509 | −16.341 | 1.00 | 40.00 | ercc | N |
| ATOM | 1527 | CA | ALA | A | 192 | 7.403 | 25.616 | −15.197 | 1.00 | 40.00 | ercc | C |
| ATOM | 1529 | CB | ALA | A | 192 | 6.300 | 24.565 | −15.182 | 1.00 | 40.00 | ercc | C |
| ATOM | 1533 | C | ALA | A | 192 | 8.780 | 24.948 | −15.184 | 1.00 | 40.00 | ercc | C |
| ATOM | 1534 | O | ALA | A | 192 | 9.183 | 24.385 | −14.170 | 1.00 | 40.00 | ercc | O |
| ATOM | 1535 | N | ASP | A | 193 | 9.497 | 25.045 | −16.304 | 1.00 | 40.00 | ercc | N |
| ATOM | 1537 | CA | ASP | A | 193 | 10.772 | 24.343 | −16.538 | 1.00 | 40.00 | ercc | C |
| ATOM | 1539 | CB | ASP | A | 193 | 11.965 | 25.098 | −15.939 | 1.00 | 40.00 | ercc | C |
| ATOM | 1542 | CG | ASP | A | 193 | 13.281 | 24.730 | −16.610 | 1.00 | 40.00 | ercc | C |
| ATOM | 1543 | OD1 | ASP | A | 193 | 13.317 | 24.630 | −17.858 | 1.00 | 40.00 | ercc | O |
| ATOM | 1544 | OD2 | ASP | A | 193 | 14.281 | 24.545 | −15.887 | 1.00 | 40.00 | ercc | O |
| ATOM | 1545 | C | ASP | A | 193 | 10.759 | 22.873 | −16.111 | 1.00 | 40.00 | ercc | C |
| ATOM | 1546 | O | ASP | A | 193 | 11.690 | 22.380 | −15.464 | 1.00 | 40.00 | ercc | O |
| ATOM | 1547 | N | CYS | A | 194 | 9.684 | 22.187 | −16.480 | 1.00 | 40.00 | ercc | N |
| ATOM | 1549 | CA | CYS | A | 194 | 9.585 | 20.753 | −16.290 | 1.00 | 40.00 | ercc | C |
| ATOM | 1551 | CB | CYS | A | 194 | 8.349 | 20.392 | −15.463 | 1.00 | 40.00 | ercc | C |
| ATOM | 1554 | SG | CYS | A | 194 | 6.787 | 20.971 | −16.149 | 1.00 | 40.00 | ercc | S |
| ATOM | 1556 | C | CYS | A | 194 | 9.560 | 20.048 | −17.640 | 1.00 | 40.00 | ercc | C |
| ATOM | 1557 | O | CYS | A | 194 | 9.168 | 20.625 | −18.657 | 1.00 | 40.00 | ercc | O |
| ATOM | 1558 | N | THR | A | 195 | 9.993 | 18.794 | −17.623 | 1.00 | 40.00 | ercc | N |
| ATOM | 1560 | CA | THR | A | 195 | 10.075 | 17.950 | −18.799 | 1.00 | 40.00 | ercc | C |
| ATOM | 1562 | CB | THR | A | 195 | 10.943 | 16.724 | −18.461 | 1.00 | 40.00 | ercc | C |
| ATOM | 1564 | OG1 | THR | A | 195 | 12.330 | 17.060 | −18.620 | 1.00 | 40.00 | ercc | O |
| ATOM | 1566 | CG2 | THR | A | 195 | 10.576 | 15.515 | −19.320 | 1.00 | 40.00 | ercc | C |
| ATOM | 1570 | C | THR | A | 195 | 8.687 | 17.497 | −19.246 | 1.00 | 40.00 | ercc | C |
| ATOM | 1571 | O | THR | A | 195 | 7.851 | 17.164 | −18.414 | 1.00 | 40.00 | ercc | O |
| ATOM | 1572 | N | LEU | A | 196 | 8.444 | 17.503 | −20.553 | 1.00 | 40.00 | ercc | N |
| ATOM | 1574 | CA | LEU | A | 196 | 7.204 | 16.960 | −21.099 | 1.00 | 40.00 | ercc | C |
| ATOM | 1576 | CB | LEU | A | 196 | 6.675 | 17.851 | −22.226 | 1.00 | 40.00 | ercc | C |
| ATOM | 1579 | CG | LEU | A | 196 | 5.162 | 17.913 | −22.483 | 1.00 | 40.00 | ercc | C |
| ATOM | 1581 | CD1 | LEU | A | 196 | 4.866 | 18.970 | −23.526 | 1.00 | 40.00 | ercc | C |
| ATOM | 1585 | CD2 | LEU | A | 196 | 4.563 | 16.579 | −22.915 | 1.00 | 40.00 | ercc | C |
| ATOM | 1589 | C | LEU | A | 196 | 7.461 | 15.550 | −21.621 | 1.00 | 40.00 | ercc | C |
| ATOM | 1590 | O | LEU | A | 196 | 8.390 | 15.329 | −22.391 | 1.00 | 40.00 | ercc | O |
| ATOM | 1591 | N | ILE | A | 197 | 6.647 | 14.590 | −21.201 | 1.00 | 40.00 | ercc | N |
| ATOM | 1593 | CA | ILE | A | 197 | 6.822 | 13.211 | −21.657 | 1.00 | 40.00 | ercc | C |
| ATOM | 1595 | CB | ILE | A | 197 | 7.414 | 12.320 | −20.547 | 1.00 | 40.00 | ercc | C |
| ATOM | 1597 | CG1 | ILE | A | 197 | 8.904 | 12.641 | −20.362 | 1.00 | 40.00 | ercc | C |
| ATOM | 1600 | CG2 | ILE | A | 197 | 7.191 | 10.840 | −20.852 | 1.00 | 40.00 | ercc | C |
| ATOM | 1604 | CD1 | ILE | A | 197 | 9.801 | 12.385 | −21.587 | 1.00 | 40.00 | ercc | C |
| ATOM | 1608 | C | ILE | A | 197 | 5.536 | 12.622 | −22.223 | 1.00 | 40.00 | ercc | C |
| ATOM | 1609 | O | ILE | A | 197 | 4.454 | 12.887 | −21.712 | 1.00 | 40.00 | ercc | O |
| ATOM | 1610 | N | LEU | A | 198 | 5.672 | 11.818 | −23.277 | 1.00 | 40.00 | ercc | N |
| ATOM | 1612 | CA | LEU | A | 198 | 4.515 | 11.323 | −24.021 | 1.00 | 40.00 | ercc | C |
| ATOM | 1614 | CB | LEU | A | 198 | 4.514 | 11.882 | −25.448 | 1.00 | 40.00 | ercc | C |
| ATOM | 1617 | CG | LEU | A | 198 | 4.007 | 13.309 | −25.656 | 1.00 | 40.00 | ercc | C |
| ATOM | 1619 | CD1 | LEU | A | 198 | 5.133 | 14.324 | −25.467 | 1.00 | 40.00 | ercc | C |
| ATOM | 1623 | CD2 | LEU | A | 198 | 3.409 | 13.437 | −27.054 | 1.00 | 40.00 | ercc | C |
| ATOM | 1627 | C | LEU | A | 198 | 4.378 | 9.802 | −24.064 | 1.00 | 40.00 | ercc | C |
| ATOM | 1628 | O | LEU | A | 198 | 5.172 | 9.105 | −24.702 | 1.00 | 40.00 | ercc | O |
| ATOM | 1629 | N | ALA | A | 199 | 3.352 | 9.308 | −23.376 | 1.00 | 40.00 | ercc | N |
| ATOM | 1631 | CA | ALA | A | 199 | 2.956 | 7.908 | −23.435 | 1.00 | 40.00 | ercc | C |
| ATOM | 1633 | CB | ALA | A | 199 | 2.904 | 7.315 | −22.044 | 1.00 | 40.00 | ercc | C |
| ATOM | 1637 | C | ALA | A | 199 | 1.593 | 7.815 | −24.111 | 1.00 | 40.00 | ercc | C |
| ATOM | 1638 | O | ALA | A | 199 | 0.914 | 8.823 | −24.268 | 1.00 | 40.00 | ercc | O |
| ATOM | 1639 | N | TRP | A | 200 | 1.198 | 6.609 | −24.503 | 1.00 | 40.00 | ercc | N |
| ATOM | 1641 | CA | TRP | A | 200 | −0.013 | 6.407 | −25.298 | 1.00 | 40.00 | ercc | C |
| ATOM | 1643 | CB | TRP | A | 200 | 0.361 | 6.163 | −26.761 | 1.00 | 40.00 | ercc | C |
| ATOM | 1646 | CG | TRP | A | 200 | 0.983 | 7.358 | −27.433 | 1.00 | 40.00 | ercc | C |
| ATOM | 1647 | CD1 | TRP | A | 200 | 2.199 | 7.924 | −27.156 | 1.00 | 40.00 | ercc | C |
| ATOM | 1649 | CD2 | TRP | A | 200 | 0.421 | 8.124 | −28.501 | 1.00 | 40.00 | ercc | C |
| ATOM | 1650 | NE1 | TRP | A | 200 | 2.421 | 9.000 | −27.979 | 1.00 | 40.00 | ercc | N |
| ATOM | 1652 | CE2 | TRP | A | 200 | 1.347 | 9.145 | −28.816 | 1.00 | 40.00 | ercc | C |
| ATOM | 1653 | CE3 | TRP | A | 200 | −0.778 | 8.050 | −29.223 | 1.00 | 40.00 | ercc | C |
| ATOM | 1655 | CZ2 | TRP | A | 200 | 1.111 | 10.084 | −29.825 | 1.00 | 40.00 | ercc | C |
| ATOM | 1657 | CZ3 | TRP | A | 200 | −1.011 | 8.983 | −30.226 | 1.00 | 40.00 | ercc | C |
| ATOM | 1659 | CH2 | TRP | A | 200 | −0.070 | 9.987 | −30.516 | 1.00 | 40.00 | ercc | C |
| ATOM | 1661 | C | TRP | A | 200 | −0.854 | 5.254 | −24.759 | 1.00 | 40.00 | ercc | C |
| ATOM | 1662 | O | TRP | A | 200 | −2.013 | 5.078 | −25.145 | 1.00 | 40.00 | ercc | O |
| ATOM | 1663 | N | SER | A | 201 | −0.254 | 4.476 | −23.863 | 1.00 | 40.00 | ercc | N |
| ATOM | 1665 | CA | SER | A | 201 | −0.935 | 3.384 | −23.185 | 1.00 | 40.00 | ercc | C |
| ATOM | 1667 | CB | SER | A | 201 | −0.461 | 2.036 | −23.741 | 1.00 | 40.00 | ercc | C |
| ATOM | 1670 | OG | SER | A | 201 | 0.846 | 1.715 | −23.292 | 1.00 | 40.00 | ercc | O |
| ATOM | 1672 | C | SER | A | 201 | −0.651 | 3.486 | −21.686 | 1.00 | 40.00 | ercc | C |
| ATOM | 1673 | O | SER | A | 201 | 0.283 | 4.187 | −21.286 | 1.00 | 40.00 | ercc | O |
| ATOM | 1674 | N | PRO | A | 202 | −1.455 | 2.797 | −20.849 | 1.00 | 40.00 | ercc | N |
| ATOM | 1675 | CA | PRO | A | 202 | −1.149 | 2.749 | −19.418 | 1.00 | 40.00 | ercc | C |
| ATOM | 1677 | CB | PRO | A | 202 | −2.327 | 1.965 | −18.825 | 1.00 | 40.00 | ercc | C |
| ATOM | 1680 | CG | PRO | A | 202 | −3.396 | 1.999 | −19.870 | 1.00 | 40.00 | ercc | C |
| ATOM | 1683 | CD | PRO | A | 202 | −2.676 | 2.037 | −21.173 | 1.00 | 40.00 | ercc | C |
| ATOM | 1686 | C | PRO | A | 202 | 0.160 | 2.002 | −19.167 | 1.00 | 40.00 | ercc | C |
| ATOM | 1687 | O | PRO | A | 202 | 0.843 | 2.260 | −18.174 | 1.00 | 40.00 | ercc | O |
| ATOM | 1688 | N | GLU | A | 203 | 0.487 | 1.084 | −20.076 | 1.00 | 40.00 | ercc | N |
| ATOM | 1690 | CA | GLU | A | 203 | 1.735 | 0.331 | −20.043 | 1.00 | 40.00 | ercc | C |
| ATOM | 1692 | CB | GLU | A | 203 | 1.691 | −0.823 | −21.054 | 1.00 | 40.00 | ercc | C |
| ATOM | 1695 | CG | GLU | A | 203 | 3.063 | −1.422 | −21.372 | 1.00 | 40.00 | ercc | C |
| ATOM | 1698 | CD | GLU | A | 203 | 2.992 | −2.774 | −22.059 | 1.00 | 40.00 | ercc | C |
| ATOM | 1699 | OE1 | GLU | A | 203 | 1.918 | −3.138 | −22.587 | 1.00 | 40.00 | ercc | O |
| ATOM | 1700 | OE2 | GLU | A | 203 | 4.025 | −3.480 | −22.071 | 1.00 | 40.00 | ercc | O |
| ATOM | 1701 | C | GLU | A | 203 | 2.951 | 1.211 | −20.306 | 1.00 | 40.00 | ercc | C |
| ATOM | 1702 | O | GLU | A | 203 | 3.923 | 1.186 | −19.548 | 1.00 | 40.00 | ercc | O |
| ATOM | 1703 | N | GLU | A | 204 | 2.878 | 1.979 | −21.391 | 1.00 | 40.00 | ercc | N |
| ATOM | 1705 | CA | GLU | A | 204 | 3.977 | 2.811 | −21.867 | 1.00 | 40.00 | ercc | C |
| ATOM | 1707 | CB | GLU | A | 204 | 3.532 | 3.582 | −23.109 | 1.00 | 40.00 | ercc | C |
| ATOM | 1710 | CG | GLU | A | 204 | 4.577 | 3.663 | −24.211 | 1.00 | 40.00 | ercc | C |
| ATOM | 1713 | CD | GLU | A | 204 | 4.067 | 4.390 | −25.448 | 1.00 | 40.00 | ercc | C |
| ATOM | 1714 | OE1 | GLU | A | 204 | 3.075 | 5.139 | −25.343 | 1.00 | 40.00 | ercc | O |
| ATOM | 1715 | OE2 | GLU | A | 204 | 4.665 | 4.216 | −26.532 | 1.00 | 40.00 | ercc | O |
| ATOM | 1716 | C | GLU | A | 204 | 4.458 | 3.782 | −20.792 | 1.00 | 40.00 | ercc | C |
| ATOM | 1717 | O | GLU | A | 204 | 5.663 | 3.938 | −20.582 | 1.00 | 40.00 | ercc | O |
| ATOM | 1718 | N | ALA | A | 205 | 3.509 | 4.436 | −20.123 | 1.00 | 40.00 | ercc | N |
| ATOM | 1720 | CA | ALA | A | 205 | 3.812 | 5.317 | −19.001 | 1.00 | 40.00 | ercc | C |
| ATOM | 1722 | CB | ALA | A | 205 | 2.554 | 6.028 | −18.529 | 1.00 | 40.00 | ercc | C |
| ATOM | 1726 | C | ALA | A | 205 | 4.436 | 4.520 | −17.862 | 1.00 | 40.00 | ercc | C |
| ATOM | 1727 | O | ALA | A | 205 | 5.432 | 4.943 | −17.272 | 1.00 | 40.00 | ercc | O |
| ATOM | 1728 | N | GLY | A | 206 | 3.841 | 3.365 | −17.562 | 1.00 | 40.00 | ercc | N |
| ATOM | 1730 | CA | GLY | A | 206 | 4.382 | 2.443 | −16.567 | 1.00 | 40.00 | ercc | C |
| ATOM | 1733 | C | GLY | A | 206 | 5.829 | 2.113 | −16.872 | 1.00 | 40.00 | ercc | C |
| ATOM | 1734 | O | GLY | A | 206 | 6.656 | 2.009 | −15.961 | 1.00 | 40.00 | ercc | O |
| ATOM | 1735 | N | ARG | A | 207 | 6.129 | 1.958 | −18.160 | 1.00 | 40.00 | ercc | N |
| ATOM | 1737 | CA | ARG | A | 207 | 7.499 | 1.771 | −18.619 | 1.00 | 40.00 | ercc | C |
| ATOM | 1739 | CB | ARG | A | 207 | 7.542 | 1.507 | −20.127 | 1.00 | 40.00 | ercc | C |
| ATOM | 1742 | CG | ARG | A | 207 | 7.046 | 0.129 | −20.536 | 1.00 | 40.00 | ercc | C |
| ATOM | 1745 | CD | ARG | A | 207 | 7.923 | −0.966 | −19.938 | 1.00 | 40.00 | ercc | C |
| ATOM | 1748 | NE | ARG | A | 207 | 7.878 | −2.207 | −20.707 | 1.00 | 40.00 | ercc | N |
| ATOM | 1750 | CZ | ARG | A | 207 | 8.591 | −2.439 | −21.806 | 1.00 | 40.00 | ercc | C |
| ATOM | 1751 | NH1 | ARG | A | 207 | 9.413 | −1.512 | −22.290 | 1.00 | 40.00 | ercc | N |
| ATOM | 1754 | NH2 | ARG | A | 207 | 8.479 | −3.603 | −22.431 | 1.00 | 40.00 | ercc | N |
| ATOM | 1757 | C | ARG | A | 207 | 8.362 | 2.973 | −18.256 | 1.00 | 40.00 | ercc | C |
| ATOM | 1758 | O | ARG | A | 207 | 9.418 | 2.817 | −17.644 | 1.00 | 40.00 | ercc | O |
| ATOM | 1759 | N | TYR | A | 208 | 7.888 | 4.164 | −18.612 | 1.00 | 40.00 | ercc | N |
| ATOM | 1761 | CA | TYR | A | 208 | 8.591 | 5.414 | −18.338 | 1.00 | 40.00 | ercc | C |
| ATOM | 1763 | CB | TYR | A | 208 | 7.675 | 6.584 | −18.667 | 1.00 | 40.00 | ercc | C |
| ATOM | 1766 | CG | TYR | A | 208 | 8.278 | 7.932 | −18.401 | 1.00 | 40.00 | ercc | C |
| ATOM | 1767 | CD1 | TYR | A | 208 | 9.174 | 8.490 | −19.303 | 1.00 | 40.00 | ercc | C |
| ATOM | 1769 | CD2 | TYR | A | 208 | 7.944 | 8.652 | −17.252 | 1.00 | 40.00 | ercc | C |
| ATOM | 1771 | CE1 | TYR | A | 208 | 9.719 | 9.726 | −19.078 | 1.00 | 40.00 | ercc | C |
| ATOM | 1773 | CE2 | TYR | A | 208 | 8.478 | 9.897 | −17.014 | 1.00 | 40.00 | ercc | C |
| ATOM | 1775 | CZ | TYR | A | 208 | 9.364 | 10.431 | −17.932 | 1.00 | 40.00 | ercc | C |
| ATOM | 1776 | OH | TYR | A | 208 | 9.914 | 11.669 | −17.703 | 1.00 | 40.00 | ercc | O |
| ATOM | 1778 | C | TYR | A | 208 | 9.061 | 5.533 | −16.892 | 1.00 | 40.00 | ercc | C |
| ATOM | 1779 | O | TYR | A | 208 | 10.076 | 6.174 | −16.608 | 1.00 | 40.00 | ercc | O |
| ATOM | 1780 | N | LEU | A | 209 | 8.309 | 4.912 | −15.988 | 1.00 | 40.00 | ercc | N |
| ATOM | 1782 | CA | LEU | A | 209 | 8.585 | 4.972 | −14.556 | 1.00 | 40.00 | ercc | C |
| ATOM | 1784 | CB | LEU | A | 209 | 7.329 | 4.593 | −13.765 | 1.00 | 40.00 | ercc | C |
| ATOM | 1787 | CG | LEU | A | 209 | 6.338 | 5.690 | −13.348 | 1.00 | 40.00 | ercc | C |
| ATOM | 1789 | CD1 | LEU | A | 209 | 6.599 | 7.050 | −14.005 | 1.00 | 40.00 | ercc | C |
| ATOM | 1793 | CD2 | LEU | A | 209 | 4.901 | 5.237 | −13.588 | 1.00 | 40.00 | ercc | C |
| ATOM | 1797 | C | LEU | A | 209 | 9.761 | 4.092 | −14.152 | 1.00 | 40.00 | ercc | C |
| ATOM | 1798 | O | LEU | A | 209 | 10.655 | 4.538 | −13.430 | 1.00 | 40.00 | ercc | O |
| ATOM | 1799 | N | GLU | A | 210 | 9.748 | 2.845 | −14.618 | 1.00 | 40.00 | ercc | N |
| ATOM | 1801 | CA | GLU | A | 210 | 10.839 | 1.906 | −14.380 | 1.00 | 40.00 | ercc | C |
| ATOM | 1803 | CB | GLU | A | 210 | 10.506 | 0.542 | −14.994 | 1.00 | 40.00 | ercc | C |
| ATOM | 1806 | CG | GLU | A | 210 | 9.318 | −0.158 | −14.332 | 1.00 | 40.00 | ercc | C |
| ATOM | 1809 | CD | GLU | A | 210 | 8.484 | −0.976 | −15.303 | 1.00 | 40.00 | ercc | C |
| ATOM | 1810 | OE1 | GLU | A | 210 | 8.137 | −2.128 | −14.969 | 1.00 | 40.00 | ercc | O |
| ATOM | 1811 | OE2 | GLU | A | 210 | 8.170 | −0.472 | −16.402 | 1.00 | 40.00 | ercc | O |
| ATOM | 1812 | C | GLU | A | 210 | 12.147 | 2.454 | −14.949 | 1.00 | 40.00 | ercc | C |
| ATOM | 1813 | O | GLU | A | 210 | 13.216 | 2.232 | −14.383 | 1.00 | 40.00 | ercc | O |
| ATOM | 1814 | N | THR | A | 211 | 12.033 | 3.193 | −16.054 | 1.00 | 40.00 | ercc | N |
| ATOM | 1816 | CA | THR | A | 211 | 13.170 | 3.778 | −16.768 | 1.00 | 40.00 | ercc | C |
| ATOM | 1818 | CB | THR | A | 211 | 12.747 | 4.334 | −18.148 | 1.00 | 40.00 | ercc | C |
| ATOM | 1820 | OG1 | THR | A | 211 | 11.605 | 3.618 | −18.637 | 1.00 | 40.00 | ercc | O |
| ATOM | 1822 | CG2 | THR | A | 211 | 13.887 | 4.196 | −19.143 | 1.00 | 40.00 | ercc | C |
| ATOM | 1826 | C | THR | A | 211 | 13.850 | 4.912 | −16.003 | 1.00 | 40.00 | ercc | C |
| ATOM | 1827 | O | THR | A | 211 | 15.079 | 4.998 | −15.986 | 1.00 | 40.00 | ercc | O |
| ATOM | 1828 | N | TYR | A | 212 | 13.053 | 5.785 | −15.389 | 1.00 | 40.00 | ercc | N |
| ATOM | 1830 | CA | TYR | A | 212 | 13.600 | 6.942 | −14.682 | 1.00 | 40.00 | ercc | C |
| ATOM | 1832 | CB | TYR | A | 212 | 12.580 | 8.074 | −14.576 | 1.00 | 40.00 | ercc | C |
| ATOM | 1835 | CG | TYR | A | 212 | 12.673 | 9.034 | −15.733 | 1.00 | 40.00 | ercc | C |
| ATOM | 1836 | CD1 | TYR | A | 212 | 13.909 | 9.522 | −16.164 | 1.00 | 40.00 | ercc | C |
| ATOM | 1838 | CD2 | TYR | A | 212 | 11.550 | 9.452 | −16.400 | 1.00 | 40.00 | ercc | C |
| ATOM | 1840 | CE1 | TYR | A | 212 | 14.012 | 10.405 | −17.239 | 1.00 | 40.00 | ercc | C |
| ATOM | 1842 | CE2 | TYR | A | 212 | 11.653 | 10.335 | −17.471 | 1.00 | 40.00 | ercc | C |
| ATOM | 1844 | CZ | TYR | A | 212 | 12.873 | 10.807 | −17.890 | 1.00 | 40.00 | ercc | C |
| ATOM | 1845 | OH | TYR | A | 212 | 12.948 | 11.683 | −18.953 | 1.00 | 40.00 | ercc | O |
| ATOM | 1847 | C | TYR | A | 212 | 14.222 | 6.618 | −13.330 | 1.00 | 40.00 | ercc | C |
| ATOM | 1848 | O | TYR | A | 212 | 15.177 | 7.278 | −12.907 | 1.00 | 40.00 | ercc | O |
| ATOM | 1849 | N | LYS | A | 213 | 13.685 | 5.599 | −12.663 | 1.00 | 40.00 | ercc | N |
| ATOM | 1851 | CA | LYS | A | 213 | 14.334 | 5.019 | −11.491 | 1.00 | 40.00 | ercc | C |
| ATOM | 1853 | CB | LYS | A | 213 | 13.331 | 4.225 | −10.645 | 1.00 | 40.00 | ercc | C |
| ATOM | 1856 | CG | LYS | A | 213 | 13.828 | 3.869 | −9.246 | 1.00 | 40.00 | ercc | C |
| ATOM | 1859 | CD | LYS | A | 213 | 13.937 | 5.114 | −8.361 | 1.00 | 40.00 | ercc | C |
| ATOM | 1862 | CE | LYS | A | 213 | 15.272 | 5.138 | −7.612 | 1.00 | 40.00 | ercc | C |
| ATOM | 1865 | NZ | LYS | A | 213 | 15.414 | 6.330 | −6.723 | 1.00 | 40.00 | ercc | N |
| ATOM | 1869 | C | LYS | A | 213 | 15.501 | 4.131 | −11.935 | 1.00 | 40.00 | ercc | C |
| ATOM | 1870 | O | LYS | A | 213 | 16.455 | 3.926 | −11.180 | 1.00 | 40.00 | ercc | O |
| ATOM | 1871 | N | ALA | A | 214 | 15.417 | 3.612 | −13.161 | 1.00 | 40.00 | ercc | N |
| ATOM | 1873 | CA | ALA | A | 214 | 16.526 | 2.879 | −13.771 | 1.00 | 40.00 | ercc | C |
| ATOM | 1875 | CB | ALA | A | 214 | 16.028 | 1.942 | −14.862 | 1.00 | 40.00 | ercc | C |
| ATOM | 1879 | C | ALA | A | 214 | 17.562 | 3.845 | −14.336 | 1.00 | 40.00 | ercc | C |
| ATOM | 1880 | OT1 | ALA | A | 214 | 17.469 | 5.020 | −14.012 | 1.00 | 40.00 | ercc | O |
| ATOM | 1881 | OT2 | ALA | A | 214 | 18.414 | 3.398 | −15.093 | 1.00 | 40.00 | ercc | O |
| ATOM | 1882 | CA | LYS | B | 67 | −15.598 | 1.556 | −13.817 | 1.00 | 40.00 | sXPA | C |
| ATOM | 1884 | CB | LYS | B | 67 | −16.023 | 1.385 | −12.341 | 1.00 | 40.00 | sXPA | C |
| ATOM | 1887 | CG | LYS | B | 67 | −17.452 | 1.936 | −12.148 | 1.00 | 40.00 | sXPA | C |
| ATOM | 1890 | CD | LYS | B | 67 | −17.449 | 3.106 | −11.162 | 1.00 | 40.00 | sXPA | C |
| ATOM | 1893 | CE | LYS | B | 67 | −18.450 | 2.820 | −10.043 | 1.00 | 40.00 | sXPA | C |
| ATOM | 1896 | NZ | LYS | B | 67 | −18.597 | 4.108 | −9.309 | 1.00 | 40.00 | sXPA | N |
| ATOM | 1900 | C | LYS | B | 67 | −14.816 | 2.873 | −13.977 | 1.00 | 40.00 | sXPA | C |
| ATOM | 1901 | O | LYS | B | 67 | −13.598 | 2.939 | −13.806 | 1.00 | 40.00 | sXPA | O |
| ATOM | 1902 | N | LYS | B | 67 | −14.741 | 0.365 | −14.167 | 1.00 | 40.00 | sXPA | N |
| ATOM | 1906 | N | ILE | B | 68 | −15.526 | 3.929 | −14.281 | 1.00 | 40.00 | sXPA | N |
| ATOM | 1908 | CA | ILE | B | 68 | −14.868 | 5.245 | −14.429 | 1.00 | 40.00 | sXPA | C |
| ATOM | 1910 | CB | ILE | B | 68 | −14.485 | 5.369 | −15.919 | 1.00 | 40.00 | sXPA | C |
| ATOM | 1912 | CG1 | ILE | B | 68 | −13.538 | 6.588 | −16.165 | 1.00 | 40.00 | sXPA | C |
| ATOM | 1915 | CG2 | ILE | B | 68 | −15.744 | 5.484 | −16.794 | 1.00 | 40.00 | sXPA | C |
| ATOM | 1919 | CD1 | ILE | B | 68 | −13.951 | 7.845 | −15.361 | 1.00 | 40.00 | sXPA | C |
| ATOM | 1923 | C | ILE | B | 68 | −15.811 | 6.354 | −13.911 | 1.00 | 40.00 | sXPA | C |
| ATOM | 1924 | O | ILE | B | 68 | −16.630 | 6.942 | −14.609 | 1.00 | 40.00 | sXPA | O |
| ATOM | 1925 | N | ILE | B | 69 | −15.647 | 6.641 | −12.665 | 1.00 | 40.00 | sXPA | N |
| ATOM | 1927 | CA | ILE | B | 69 | −16.413 | 7.700 | −11.999 | 1.00 | 40.00 | sXPA | C |
| ATOM | 1929 | CB | ILE | B | 69 | −17.441 | 6.958 | −11.158 | 1.00 | 40.00 | sXPA | C |
| ATOM | 1931 | CG1 | ILE | B | 69 | −18.500 | 6.386 | −12.141 | 1.00 | 40.00 | sXPA | C |
| ATOM | 1934 | CG2 | ILE | B | 69 | −18.097 | 7.904 | −10.141 | 1.00 | 40.00 | sXPA | C |
| ATOM | 1938 | CD1 | ILE | B | 69 | −19.747 | 5.875 | −11.399 | 1.00 | 40.00 | sXPA | C |
| ATOM | 1942 | C | ILE | B | 69 | −15.330 | 8.490 | −11.254 | 1.00 | 40.00 | sXPA | C |
| ATOM | 1943 | O | ILE | B | 69 | −15.223 | 8.550 | −10.033 | 1.00 | 40.00 | sXPA | O |
| ATOM | 1944 | N | ASP | B | 70 | −14.471 | 9.006 | −12.094 | 1.00 | 40.00 | sXPA | N |
| ATOM | 1946 | CA | ASP | B | 70 | −13.250 | 9.767 | −11.728 | 1.00 | 40.00 | sXPA | C |
| ATOM | 1948 | CB | ASP | B | 70 | −13.286 | 10.983 | −12.643 | 1.00 | 40.00 | sXPA | C |
| ATOM | 1951 | CG | ASP | B | 70 | −11.986 | 11.777 | −12.488 | 1.00 | 40.00 | sXPA | C |
| ATOM | 1952 | OD1 | ASP | B | 70 | −11.972 | 12.705 | −11.679 | 1.00 | 40.00 | sXPA | O |
| ATOM | 1953 | OD2 | ASP | B | 70 | −11.027 | 11.437 | −13.174 | 1.00 | 40.00 | sXPA | O |
| ATOM | 1954 | C | ASP | B | 70 | −13.128 | 10.210 | −10.266 | 1.00 | 40.00 | sXPA | C |
| ATOM | 1955 | O | ASP | B | 70 | −14.085 | 10.506 | −9.564 | 1.00 | 40.00 | sXPA | O |
| ATOM | 1956 | N | THR | B | 71 | −11.890 | 10.303 | −9.859 | 1.00 | 40.00 | sXPA | N |
| ATOM | 1958 | CA | THR | B | 71 | −11.536 | 10.767 | −8.501 | 1.00 | 40.00 | sXPA | C |
| ATOM | 1960 | CB | THR | B | 71 | −10.627 | 9.679 | −7.920 | 1.00 | 40.00 | sXPA | C |
| ATOM | 1962 | OG1 | THR | B | 71 | −9.279 | 9.945 | −8.309 | 1.00 | 40.00 | sXPA | O |
| ATOM | 1964 | CG2 | THR | B | 71 | −11.040 | 8.303 | −8.455 | 1.00 | 40.00 | sXPA | C |
| ATOM | 1968 | C | THR | B | 71 | −10.767 | 12.081 | −8.657 | 1.00 | 40.00 | sXPA | C |
| ATOM | 1969 | O | THR | B | 71 | −11.367 | 13.134 | −8.760 | 1.00 | 40.00 | sXPA | O |
| ATOM | 1970 | N | GLY | B | 72 | −9.441 | 11.994 | −8.672 | 1.00 | 40.00 | sXPA | N |
| ATOM | 1972 | CA | GLY | B | 72 | −8.601 | 13.068 | −8.174 | 1.00 | 40.00 | sXPA | C |
| ATOM | 1975 | C | GLY | B | 72 | −7.139 | 12.870 | −8.522 | 1.00 | 40.00 | sXPA | C |
| ATOM | 1976 | O | GLY | B | 72 | −6.303 | 12.669 | −7.641 | 1.00 | 40.00 | sXPA | O |
| ATOM | 1977 | N | GLY | B | 73 | −6.831 | 12.927 | −9.814 | 1.00 | 40.00 | sXPA | N |
| ATOM | 1979 | CA | GLY | B | 73 | −5.456 | 13.034 | −10.265 | 1.00 | 40.00 | sXPA | C |
| ATOM | 1982 | C | GLY | B | 73 | −5.363 | 13.685 | −11.631 | 1.00 | 40.00 | sXPA | C |
| ATOM | 1983 | O | GLY | B | 73 | −6.103 | 14.621 | −11.935 | 1.00 | 40.00 | sXPA | O |
| ATOM | 1984 | N | GLY | B | 74 | −4.451 | 13.186 | −12.458 | 1.00 | 40.00 | sXPA | N |
| ATOM | 1986 | CA | GLY | B | 74 | −4.513 | 13.435 | −13.886 | 1.00 | 40.00 | sXPA | C |
| ATOM | 1989 | C | GLY | B | 74 | −4.658 | 12.159 | −14.692 | 1.00 | 40.00 | sXPA | C |
| ATOM | 1990 | O | GLY | B | 74 | −3.717 | 11.721 | −15.354 | 1.00 | 40.00 | sXPA | O |
| ATOM | 1991 | N | PHE | B | 75 | −5.844 | 11.561 | −14.635 | 1.00 | 40.00 | sXPA | N |
| ATOM | 1993 | CA | PHE | B | 75 | −6.073 | 10.256 | −15.243 | 1.00 | 40.00 | sXPA | C |
| ATOM | 1995 | CB | PHE | B | 75 | −5.188 | 9.195 | −14.585 | 1.00 | 40.00 | sXPA | C |
| ATOM | 1998 | CG | PHE | B | 75 | −5.715 | 8.699 | −13.269 | 1.00 | 40.00 | sXPA | C |
| ATOM | 1999 | CD1 | PHE | B | 75 | −5.967 | 9.581 | −12.231 | 1.00 | 40.00 | sXPA | C |
| ATOM | 2001 | CD2 | PHE | B | 75 | −5.958 | 7.350 | −13.069 | 1.00 | 40.00 | sXPA | C |
| ATOM | 2003 | CE1 | PHE | B | 75 | −6.452 | 9.127 | −11.019 | 1.00 | 40.00 | sXPA | C |
| ATOM | 2005 | CE2 | PHE | B | 75 | −6.443 | 6.890 | −11.859 | 1.00 | 40.00 | sXPA | C |
| ATOM | 2007 | CZ | PHE | B | 75 | −6.690 | 7.780 | −10.833 | 1.00 | 40.00 | sXPA | C |
| ATOM | 2009 | C | PHE | B | 75 | −7.540 | 9.851 | −15.143 | 1.00 | 40.00 | sXPA | C |
| ATOM | 2010 | O | PHE | B | 75 | −8.065 | 9.648 | −14.048 | 1.00 | 40.00 | sXPA | O |
| ATOM | 2011 | N | ILE | B | 76 | −8.196 | 9.734 | −16.293 | 1.00 | 40.00 | sXPA | N |
| ATOM | 2013 | CA | ILE | B | 76 | −9.626 | 9.314 | −16.340 | 1.00 | 40.00 | sXPA | C |
| ATOM | 2015 | CB | ILE | B | 76 | −9.912 | 9.116 | −17.822 | 1.00 | 40.00 | sXPA | C |
| ATOM | 2017 | CG1 | ILE | B | 76 | −11.255 | 9.765 | −18.132 | 1.00 | 40.00 | sXPA | C |
| ATOM | 2020 | CG2 | ILE | B | 76 | −9.939 | 7.627 | −18.202 | 1.00 | 40.00 | sXPA | C |
| ATOM | 2024 | CD1 | ILE | B | 76 | −11.046 | 11.235 | −18.457 | 1.00 | 40.00 | sXPA | C |
| ATOM | 2028 | C | ILE | B | 76 | −9.805 | 8.040 | −15.541 | 1.00 | 40.00 | sXPA | C |
| ATOM | 2029 | O | ILE | B | 76 | −9.332 | 6.993 | −15.908 | 1.00 | 40.00 | sXPA | O |
| ATOM | 2030 | N | LEU | B | 77 | −10.428 | 8.124 | −14.419 | 1.00 | 40.00 | sXPA | N |
| ATOM | 2032 | CA | LEU | B | 77 | −10.538 | 6.903 | −13.585 | 1.00 | 40.00 | sXPA | C |
| ATOM | 2034 | CB | LEU | B | 77 | −10.248 | 7.384 | −12.158 | 1.00 | 40.00 | sXPA | C |
| ATOM | 2037 | CG | LEU | B | 77 | −9.740 | 6.233 | −11.261 | 1.00 | 40.00 | sXPA | C |
| ATOM | 2039 | CD1 | LEU | B | 77 | −10.790 | 5.899 | −10.214 | 1.00 | 40.00 | sXPA | C |
| ATOM | 2043 | CD2 | LEU | B | 77 | −9.429 | 4.966 | −12.072 | 1.00 | 40.00 | sXPA | C |
| ATOM | 2047 | C | LEU | B | 77 | −11.903 | 6.266 | −13.665 | 1.00 | 40.00 | sXPA | C |
| ATOM | 2048 | OT1 | LEU | B | 77 | −12.026 | 5.282 | −14.383 | 1.00 | 40.00 | sXPA | O |
| ATOM | 2049 | OT2 | LEU | B | 77 | −12.787 | 6.747 | −12.981 | 1.00 | 40.00 | sXPA | O |
| END | | | | | | | | | | | | |

We claim:

1. An XPA fragment of up to about 35 amino acids which comprises SEQ ID NO:1 from amino acid 70 to amino acid 78.

2. The XPA fragment of claim 1, which consists essentially of SEQ ID NO:1 from about amino acid 67 to about amino acid 80.

3. The XPA fragment of claim 1, which consists essentially of SEQ ID NO:1 from about amino acid 59 to about amino acid 93.

4. A complex of ECCR1 or a fragment thereof and an XPA fragment, wherein the XPA fragment is up to about 35 amino acids and comprises SEQ ID NO:1 from amino acid 70 to amino acid 78.

5. The complex of claim 4, wherein the XPA fragment consists essentially of SEQ ID NO:1 from about amino acid 67 to about amino acid 80.

6. The complex of claim 4, wherein the XPA fragment consists essentially of SEQ ID NO:1 from about amino acid 59 to about amino acid 93.

7. The complex of claim 4, which comprises a fragment of ERCC1 comprising SEQ ID NO:2 from about amino acid 96 to about amino acid 119.

8. The complex of claim 7, which comprises a fragment of ERCC1 consisting essentially of SEQ ID NO:2 from about amino acid 92 to about amino acid 214.

9. The complex of claim 7, which comprises a fragment of ERCC1 consisting essentially of SEQ ID NO:2 from about amino acid 96 to about amino acid 214.

10. A crystal of a complex of an XPA peptide and an ERCC1 peptide, wherein the crystal effectively diffracts X-rays for the determination of the atomic coordinates of the complex.

11. The crystal of claim 10, wherein the atomic coordinates of the binding site of the complex can be determined to a resolution of better than about 5.0 Angstroms.

12. The crystal of claim 10, wherein the atomic coordinates of the binding site of the complex can be determined to a resolution of better than about 4.0 Angstroms.

13. The crystal of claim 10, wherein the atomic coordinates of the binding site of the complex can be determined to a resolution of better than about 3.0 Angstroms.

14. The crystal of claim 10, wherein the crystal belongs to space group I4₁32 and has unit cell dimensions a=b=c=128.6 Å.

15. The crystal of claim 10, wherein the complex has the atomic coordinates of Table 2.

16. The crystal of claim 10, wherein the XPA-ERCC1 complex comprises an XPA peptide consisting of amino acids 67 to 80 of SEQ ID NO:1 and an ERCC1 peptide consisting of amino acids 92 to 214 of SEQ ID NO:2.

17. A method for preparing a crystal of an XPA-ERCC1 complex, which comprises incubating a mixture comprising an XPA central domain peptide and an ERCC1 central domain peptide in a closed container over a reservoir solution comprising a precipitant under conditions suitable for crystallization until the crystal forms.

18. The method of claim 17, wherein the mixture comprises Tris buffer.

19. The method of claim 17, wherein the mixture comprises ammonium dihydrogen phosphate.

20. The method of claim 19, wherein the mixture further comprises glycerol.

21. The method of claim 17, wherein the reservoir solution comprises ammonium dihydrogen phosphate.

22. A crystal of a complex of an XPA peptide and an ERCC1 peptide, which is produced by a method which comprises incubating a mixture comprising an XPA central domain peptide, an ERCC1 central domain peptide, a buffer, and a precipitant, over a reservoir solution comprising a precipitant, in a closed container, under conditions suitable for crystallization, until a crystal forms.

23. A method of determining whether a compound inhibits the formation of an XPA/ERCC1 complex (inhibits NER activity), which comprises:
contacting the compound with an ERCC1 polypeptide that binds to XPA and an XPA polypeptide of up to about 35 amino acids which comprises SEQ ID NO:1 from amino acid 70 to amino acid 78 under conditions in which a complex of the XPA and ERCC1 polypeptides can form in the absence of the compound; and
measuring the binding of the ERCC1 polypeptide with the XPA polypeptide; wherein a compound is identified as an inhibitor of complex formation when there is a decrease in the binding of the ERCC1 polypeptide with the XPA polypeptide in the presence but not the absence of the test compound.

24. The method of claim 23, wherein the test compound is a small molecule.

25. The method of claim 23, wherein the test compound is a peptide.

26. The method of claim 25, wherein the peptide has a conformationally constrained loop which comprises the sequence Gly-Gly-Gly.

27. The method of claim 25, wherein the peptide has a conformationally constrained loop which comprises the sequence Gly-Gly-Gly-Phe.

28. A method of identifying a compound that inhibits the formation of an XPA/ERCC1 complex (inhibits NER activity) comprising:
(a) providing structural coordinates defining all or a portion of the three-dimensional structure of the XPA binding site of ERCC1;
(b) providing structural coordinates of the compound; and
(c) fitting the structural coordinates of the compound to structural coordinates of the XPA binding site of ERCC1.

29. The method of claim 28, wherein the structural coordinates comprise all or a portion of the coordinates of Table 1.

30. The method of claim 28, wherein structural coordinates comprise amino acid residues Asn110, Ser142, Tyr145, Tyr152.

31. The method of claim 30, wherein structural coordinates further comprise amino acid residues Arg144, and Leu148.

32. The method of claim 30, wherein structural coordinates further comprise amino acid residues Arg106, and Phe140.

33. The method of claim 30, wherein structural coordinates further comprise amino acid residues Asn129, and Arg156.

34. The method of claim 28, wherein the structural coordinates defining the three-dimensional structure of the binding region of an XPA/ERCC1 complex are provided by X-ray crystallography.

35. The method of claim 28, wherein the structural coordinates defining the three-dimensional structure of the binding region of an XPA/ERCC1 complex are provided by NMR.

36. The method of claim 28, wherein the structural coordinates defining the three-dimensional structure of the binding region of an XPA/ERCC1 complex are provided by X-ray crystallography and NMR.

37. The method of claim 28, which further comprises obtaining or synthesizing the compound and determining the ability of the compound to inhibit binding of XPA to ERCC1.

38. A method of identifying a compound that inhibits the formation of an XPA/ERCC1 complex comprising:
(a) providing structural coordinates defining all or a portion of XPA when complexed with ERCC1;
(b) providing structural coordinates of the compound; and
(c) fitting the structural coordinates of the compound to XPA structural coordinates.

39. The method of claim 38, wherein structural coordinates are provided for at least two XPA amino acids that contact the XPA binding site of ERCC1.

40. The method of claim 39, wherein the at least two XPA amino acids are selected from the group consisting of Thr71, Gly72, Gly73, Gly74, Phe75, and Ile76.

41. The method of claim 39, wherein structural coordinates are provided for at least XPA amino acids Gly72, Gly73, Gly74, and Phe75.

42. The method of claim 28, wherein atoms of the compound that contact ERCC1 when the compound is crystallized with a portion of the ERCC1 peptide have locations that correspond to atoms of XPA that contact ERCC1.

43. The method of claim 28 or 38, wherein the fitting of step (c) is assisted by computer.

44. The method of claim 28 or 38, which further comprises obtaining or synthesizing the compound and determining the ability of the compound to inhibit NER activity.

45. A computer-assisted method for identifying a compound that inhibits NER activity comprising a processor, a data storage system, an input device, and an output device, comprising:
(a) inputting into the programmed computer through said input device data comprising the three-dimensional coordinates of a subset of the atoms of an XPA-ERCC1 complex as set out in Table 1;
(b) providing a database of chemical and peptide structures stored in said computer data storage system;
(c) selecting from the database, using computer methods, structures having a portion that is structurally similar to the criteria data set;
(d) outputting to the output device the selected chemical structures having a portion similar to the criteria data set; and
(e) determining whether the compound inhibits NER activity.

46. An inhibitor of XPA-ERCC1 complex formation identified by:
(a) providing structural coordinates defining all or a portion of the three-dimensional structure of the XPA binding site of ERCC1;
(b) providing structural coordinates of the inhibitor; and
(c) fitting the structure of the inhibitor to structural coordinates of the XPA binding site of ERCC1.

47. An inhibitor of XPA-ERCC1 complex formation identified by:
(a) providing structural coordinates defining all or a portion of the three-dimensional structure of the XPA when complexed with ERCC1;
(b) providing structural coordinates of the inhibitor; and
(c) fitting the structure of the inhibitor to the XPA structural coordinates.

48. The inhibitor of claim 46 or 47, which binds to at least two amino acids of ERCC1 selected from the group consisting of Asn110, Ser142, Tyr145, Tyr152.

49. A method of inhibiting NER activity comprising administering an effective amount of the compound of any one of claims 46 to 48.

50. A method of inhibiting tumor growth in a mammal comprising administering a therapeutically effective amount of the compound of any one of claims 46 to 48.

51. A method of treating a hyperproliferative disease comprising administering a therapeutically effective amount of the compound of any one of claims 46 to 48.

52. The method of claim 50 or 51, wherein the compound is administered in combination with an anti-neoplastic agent.

53. The method of claim 52, wherein the antineoplastic agent is a DNA damaging agent.

54. The method of claim 52, wherein the antineoplastic agent is a chemotherapeutic agent.

55. The method of claim 52, wherein the antineoplastic agent is a DNA alkylating agent.

56. The method of claim 52, wherein the antineoplastic agent is a platinating agent.

57. The method of claim 52, wherein the antineoplastic agent is a topoisomerase inhibitor.

58. The method of claim 52, wherein the antineoplastic agent is radiation.


SEQUENCE LISTING (9 sequences)

SEQ ID NO: 1 - protein, 273 aa, Homo sapiens
MAAADGALPEAAALEQPAELPASVRASIERKRQRALMLRQARLAARPYSATAAAATGGMANVKAAPKIIDTGGGFILEEEEEEEQKIGKVVHQPGPVMEFDYVICEECGKEFMDSYLMNHFDLPTCDNCRDADDKHKLITKTEAKQEYLLKDCDLEKREPPLKFIVKKNPHHSQWGDMKLYLKLQIVKRSLEVWGSQEALEEAKEVRQENREKMKQKKFDKKVKELRRAVRSSVWKRETIVHQHEYGPEENLEDDMYRKTCTMCGHELTYEKM

SEQ ID NO: 2 - protein, 297 aa, Homo sapiens
MDPGKDKEGVPQPSGPPARKKFVIPLDEDEVPPGVAKPLFRSTQSLPTVDTSAQAAPQTYAEYAISQPLEGAGATCPTGSEPLAGETPNQALKPGAKSNSIIVSPRQRGNPVLKFVRNVPWEFGDVIPDYVLGQSTCALFLSLRYHNLHPDYIHGRLQSLGKNFALRVLLVQVDVKDPQQALKELAKMCILADCTLILAWSPEEAGRYLETYKAYEQKPADLLMEKLEQDFVSRVTECLTTVKSVNKTDSQTLLTTFGSLEQLIAASREDLALCPGLGPQKARRLFDVLHEPFLKVP

SEQ ID NO: 3 - protein, 14 aa, Unknown
  Synthetic His tag
MGSSHHHHHHSQDP

SEQ ID NO: 4 - DNA, 37 bp, unknown
  synthetic
gacacaggaggaggcgccattctagaagaggaagaag

SEQ ID NO: 5 - DNA, 34 bp, unknown
  synthetic
gacacaggaggattcattctagaagaggaagaag

SEQ ID NO: 6 - DNA, 38 bp, unknown
  synthetic
gataattgacacaggattcattctagaagaggaagaag

SEQ ID NO: 7 - DNA, 46 bp, unknown
  synthetic
gccagcgctcggttttttttttttttttttttttccgagcgctggc

SEQ ID NO: 8 - DNA, 35 bp, unknown
  synthetic
gggggaagagtgcacagaagaagacctggtcgacc

SEQ ID NO: 9 - DNA, 40 bp, unknown
  synthetic
ccggtggccagcgctcggcgtttttttttttttttttttt